(19) United States
(12) Patent Application Publication
(10) Pub. No.: US 2012/0148574 A1
(43) Pub. Date: Jun. 14, 2012

(54) Diagnostic Markers for Ankylosing Spondylitis

(75) Inventors: Matthew Arthur Brown, Fig Tree Pocket (AU); John Duffin Reveille, Houston, TX (US); Bryan Paul Wordsworth, Oxford (GB)

(73) Assignees: BOARD OF REGENTS OF THE UNIVERSITY OF TEXAS SYSTEM, Austin, TX (US); THE UNIVERSITY OF QUEENSLAND, St. Lucia, QL (AU)

(21) Appl. No.: 13/122,366

(22) PCT Filed: Oct. 2, 2009
(86) PCT No.: PCT/AU09/01320

§ 371 Date: Sep. 26, 2011

(30) Foreign Application Priority Data
Oct. 2, 2008 (AU) 200805151
Oct. 2, 2008 (AU) 2008905149

(51) Int. Cl. A61K 39/395; C40B 30/04; C12Q 1/68

(52) U.S. Cl. 424/133.1; 435/6.12; 435/6.11; 435/6.19; 506/9

(57) ABSTRACT

The present invention discloses methods and agents for diagnosing the presence or risk of development of ankylosing spondylitis (AS) in mammals, which are based on the detection of polymorphisms within any one or more of the ARTS-1 gene, the IL-23R gene, the TNFR1 gene locus, the TRADD gene locus, the IL-1R1 gene locus, the IL-1R2 gene locus, the CD74 gene locus and the chromosome loci 2P15, 2Q31.3 and 4Q13.1. The present invention also features methods for the treatment or prevention of AS based on the diagnostic methods.

### FIELD OF THE INVENTION

This invention relates generally to methods and agents for diagnosing the presence or risk of development of ankylosing spondylitis (AS) in mammals. The methods and agents are based on the detection of polymorphisms within any one or more of the ARTS-1 gene, the IL-23R gene, the TNFR1 gene locus, the TRADD gene locus, the IL-1R1 gene locus, the IL-1R2 gene locus, the CD74 gene locus and the chromosome loci 2P15, 2Q31.3 and 4Q13.1. The invention also features methods for the treatment or prevention of AS based on the diagnostic methods of the present invention.

### BACKGROUND OF THE INVENTION

AS affects 1-9 per 1000 Caucasian individuals, making it one of the most common causes of inflammatory arthritis (Van der Linden, S. et al., 1983, Br J Rheumatol, 22: 18-19 and; Braun, J. et al., 1998, Arthritis Rheum, 41: 58-67). The condition principally affects the axial skeleton including the spine and sacroiliac joints, causing pain, stiffness, and eventually bony ankylosis. Peripheral joints and tendon insertions (entheses) are commonly affected, and approximately one-third of patients develop acute anterior uveitis.

Genetic factors play a major role in the pathogenesis of AS (Brown, M. A. et al., 1997, Arthritis Rheum, 40: 1823-1828) and there is a striking tendency towards familial clustering and a connection with human leukocyte antigen (HLA)-B27 (Reville, J. D., 2006, Current Opinion in Rheumatology 18: 332-341). The major susceptibility gene, HLA-B27, is present in about 90% of Caucasians with AS, yet only 1-5% of HLA-B27 carriers develop AS, and HLA-B27 carriage alone does not explain the pattern of disease recurrence in families, (Brown, M. A. et al., 2000, Ann Rheum Dis, 59: 883-886).

Current genetic methods for determining the risk of developing AS or diagnosing subjects with AS rely on detecting the presence of the HLA-B27 gene. However, as discussed above, this screening method is extremely unreliable since a large proportion of subjects who carry the HLA-B27 gene never develop AS.

Accordingly, there is a recognized need for more effective genetic markers for detecting the presence or diagnosing the risk of AS. It would be highly advantageous to have a reliable screening method to enable better treatment and management decisions to be made in subjects with AS or a predisposition to developing AS.

### SUMMARY OF THE INVENTION

The present invention is predicated in part on the discovery that (1) polymorphisms within the IL-1R1 gene locus, the IL-1R2 gene locus, the CD74 gene and the chromosome loci 2Q31.3 and 4Q13.1, as well as (2) certain polymorphisms within the ARTS-1 and IL-23R genes, the TNFR1 gene locus, the TRADD gene locus and the chromosome locus 2P15, are surrogate markers for AS.

Accordingly, in one aspect, the present invention provides methods for diagnosing the presence or risk of development of AS in a subject. In certain embodiments, these methods comprise (a) obtaining from the subject a biological sample comprising at least a portion of an AS marker selected from an IL-1R1 gene locus, an IL-1R2 gene locus, a CD74 gene locus, a 2Q31.3 chromosome locus and a 4Q13.1 chromosome locus or an expression product thereof; and (b) analyzing the sample for a polymorphism in the AS marker, which is indicative of the presence or risk of development of AS.

In some embodiments, the sample is analyzed for the presence of a polymorphism in the IL-1R1 gene locus. Suitably, the analysis comprises determining the identity of a polymorphic nucleotide in a polymorphic site within the IL-1R1 gene locus, having reference sequence number rs949963 on chromosome 2. In illustrative examples of this type, the presence of C (cytosine) at rs949963, indicates that the subject has AS or is at risk of developing AS.

In some embodiments, the sample is analyzed for the presence of a polymorphism in the IL-1R2 gene locus. Generally, the analysis comprises determining the identity of a polymorphic nucleotide in a polymorphic site within the IL-1R2 gene locus, having reference sequence number rs2310173 on chromosome 2. In illustrative examples of this type, the presence of T (thymine) at rs2310173, indicates that the subject has AS or is at risk of developing AS.

In some embodiments, the sample is analyzed for the presence of a polymorphism in the CD74 gene locus or in a gene genetically linked thereto. Suitably, the analysis comprises determining the identity of a polymorphic nucleotide in a polymorphic site that is in genetic linkage with the CD74 gene, e.g., a polymorphic site having reference sequence number rs15251 on chromosome 5, which is located within the Treacher Collins-Franceschetti syndrome 1 (TCOF1) gene. Suitably, the presence of C at rs 15251, indicates that the subject has AS or is at risk of developing AS. In illustrative examples of this type, the presence of C instead of T at rs15251 changes the corresponding amino acid residue at residue 1313 of the TCOF1 polypeptide (as set forth for example in GenPept Accession No. NP_{—}000347 [GI:57164975]) from valine (Val) to alanine (Ala), which indicates that the subject has AS or is at risk of developing AS. Accordingly, in some embodiments, the sample is analyzed for the presence of Val at residue 1313 of the TCOF1 polypeptide, which indicates that the subject has AS or is at risk of developing AS.

In some embodiments, the sample is analyzed for the presence of a polymorphism in the 2Q31.3 chromosome locus. Generally, the analysis comprises determining the identity of a polymorphic nucleotide in a polymorphic site within the 2Q31.3 chromosome locus, having reference sequence number rs1018326 on chromosome 2. In illustrative examples of this type, the presence of C at rs1018326, indicates that the subject has AS or is at risk of developing AS. In accordance with the present invention, the rs1018326 polymorphic site is considered to be in genetic linkage with the UBE2E3 gene and consequently, in some embodiments, the analysis for the presence of a polymorphism in the 2Q31.3 chromosome locus comprises analyzing the presence of a polymorphism in the UBE2E3 gene.

In some embodiments, the sample is analyzed for the presence of a polymorphism in the 4Q13.1 chromosome locus. Suitably, the analysis comprises determining the identity of a polymorphic nucleotide in a polymorphic site within the 4Q13.1 chromosome locus, having reference sequence number rs10517820 on chromosome 4. In illustrative examples of this type, the presence of G at rs10517820, indicates that the subject has AS or is at risk of developing AS.

Suitably, the sample is analyzed for the presence of a polymorphism in a single AS marker as broadly defined above, which is indicative of the presence or risk of development of AS. However, in certain embodiments, it is desirable to analyze the sample for the presence of a polymorphism in at least 2, 3, 4 or all 5 AS markers as broadly described above, which are indicative of the presence or risk of development of AS. For example, the sample may be analyzed for the presence of a polymorphism in at least two AS markers as broadly described above, illustrative combinations of which include: (1) a polymorphism in the IL-1R1 gene locus and a polymorphism in the IL-1R2 gene locus; (2) a polymorphism in the IL-1R1 gene locus and a polymorphism in the CD74 gene locus; (3) a polymorphism in the IL-1R1 gene locus and a polymorphism in the 2Q31.3 chromosome locus; (4) a polymorphism in the IL-1R1 gene locus and a polymorphism in the 4Q13.1 chromosome locus; (5) a polymorphism in the IL-1R2 gene locus and a polymorphism in the CD74 gene locus; (6) a polymorphism in the IL-1R2 gene locus and a polymorphism in the 2Q31.3 chromosome locus; (7) a polymorphism in the IL-1R2 gene locus and a polymorphism in the 4Q13.1 chromosome locus; (8) a polymorphism in the CD74 gene locus and a polymorphism in the 2Q31.3 chromosome locus; (9) a polymorphism in the CD74 gene locus and a polymorphism in the 4Q13.1 chromosome locus; (10) a polymorphism in the 2Q31.3 chromosome locus and a polymorphism in the 4Q13.1 chromosome locus; (11) a polymorphism in the IL-1R1 gene locus and a polymorphism in the IL-1R2 gene locus and a polymorphism in the CD74 gene locus; (12) a polymorphism in the IL-1R1 gene locus and a polymorphism in the IL-1R2 gene locus and a polymorphism in the 2Q31.3 chromosome locus; (13) a polymorphism in the IL-1R1 gene locus and a polymorphism in the IL-1R2 gene locus and t a polymorphism in the 4Q13.1 chromosome locus; (14) a polymorphism in the IL-1R2 gene locus and a polymorphism in the CD74 gene locus and a polymorphism in the 2Q31.3 chromosome locus; (15) a polymorphism in the IL-1R2 gene locus and a polymorphism in the CD74 gene locus and a polymorphism in the 4Q13.1 chromosome locus; (16) a polymorphism in the CD74 gene locus and a polymorphism in the 2Q31.3 chromosome locus and a polymorphism in the 4Q13.1 chromosome locus; (17) a polymorphism in the IL-1R1 gene locus and a polymorphism in the IL-1R2 gene locus and a polymorphism in the CD74 gene locus and a polymorphism in the 2Q31.3 chromosome locus; (18) a polymorphism in the IL-1R1 gene locus and a polymorphism in the IL-1R2 gene locus and a polymorphism in the CD74 gene locus and a polymorphism in the 4Q13.1 chromosome locus; (19) a polymorphism in the IL-1R2 gene locus and a polymorphism in the CD74 gene locus and a polymorphism in the 2Q31.3 chromosome locus and a polymorphism in the 4Q13.1 chromosome locus. In still other embodiments, the sample is analyzed for the presence a polymorphism in each of the five AS markers as broadly described above.

In some embodiments, the methods further comprise analyzing the sample for the presence of a polymorphism in at least one other AS marker. For example, other AS markers may be selected from an ARTS-1 gene, an IL-23R gene, a TNFR1 gene locus, a TRADD gene locus, a 2P15 chromosomal locus, 21Q22 chromosomal locus and a HLA-B27 gene, as disclosed for example in International Application No. PCT/AU2008/000762 filed 29 May 2008 (WO 2008/144827). Accordingly, in some embodiments, the sample is further analyzed for the presence of a polymorphism in the ARTS-1 gene, wherein the analysis comprises determining the identity of a polymorphic nucleotide in at least one polymorphic site within the ARTS-1 gene, having a reference sequence number on chromosome 5 selected from the group consisting of rs27044, rs17482078, rs10050860, rs30187 and rs2287987. In illustrative examples of this type, the presence of G (guanine) at rs27044; T (thymine) at rs30187 or rs2287987; or C (cytosine) at rs17482078 or rs10050860 indicates that the subject has AS or is at risk of developing AS. In this regard, the presence of G instead of C at rs27044 changes the corresponding amino acid residue at residue 730 of the ARTS-1 polypeptide (as set forth for example in GenPept Accession No. NP_{—}057526 [GI:94818901] or SEQ ID NO: 2 of WO 2008/144827) from glutamic acid (Glu) to glutamine (Gln); or the presence of C instead of T at rs17482078 changes the corresponding amino acid residue at residue 725 of the ARTS-1 polypeptide from Gln to arginine (Arg); or the presence of C instead of T at rs10050860 changes the corresponding amino acid residue at residue 575 of the ARTS-1 polypeptide from asparagine (Asn) to aspartic acid (Asp); or the presence of T instead of C at rs2287987 changes the corresponding amino acid residue at residue 349 of the ARTS-1 polypeptide from valine (Val) to methionine (Met); or the presence of T instead of C at rs30187 changes the corresponding amino acid reside at residue 528 of the ARTS-1 polypeptide from Arg to lysine (Lys), which indicates that the subject has AS or is at risk of developing AS. Accordingly, in some embodiments, the sample is analyzed for the presence of Gln at residue 730; or the presence of Arg at residue 725; or the presence of Asp at residue 575; or the presence of Met at residue 349; or the presence of Lys at residue 528, of the ARTS-1 polypeptide, which indicates that the subject has AS or is at risk of developing AS.

In some embodiments, the sample is further analyzed for the presence of a polymorphism in the IL-23R gene, wherein the analysis comprises determining the identity of a polymorphic nucleotide in at least one polymorphic site within the IL-23R gene having a reference sequence number on chromosome 1 selected from the group consisting of rs1004819, rs10489629, rs11465804, rs11209026, rs1343151, rs10889677, rs11209032 and rs1495965. In representative examples of this type, the presence of T (thymine) at rs11465804 or rs10489629; G (guanine) at rs11209026 or rs1343151; C (cytosine) at rs1495965; or A (adenine) at rs1004819, rs10889677 or rs11209032, indicates that the subject has AS or is at risk of developing AS. In embodiments in which G is present at rs11209026 instead of A, the corresponding amino acid at residue 381 of the IL23R polypeptide (as set forth for example in GenPept Accession No. NP_{—}653302 [GI:24430212] or SEQ ID NO: 4 WO 2008/144827) changes from Gln to Arg. Accordingly, in some embodiments, the sample is analyzed for the presence of Arg at residue 381 of the IL23R polypeptide, which indicates that the subject has AS or is at risk of developing AS.

In some embodiments, the sample is further analyzed for the presence of a polymorphism in the TNFR1 gene locus, wherein the analysis comprises determining the identity of a polymorphic nucleotide in at least one polymorphic site within the TNFR1 gene locus, having reference sequence number rs4149576 on chromosome 12. In illustrative examples of this type, the presence of C (cytosine) at rs4149576 indicates that the subject has AS or is at risk of developing AS.

In some embodiments, the sample is further analyzed for the presence of a polymorphism in the TRADD gene locus, wherein the analysis comprises determining the identity of a polymorphic nucleotide in at least one polymorphic site within that locus, having reference sequence number rs9033 on chromosome 16. In illustrative examples of this type, the presence of G (guanine) at rs9033 indicates that the subject has AS or is at risk of developing AS.

In some embodiments, the sample is further analyzed for the presence of a polymorphism in the 2P15 chromosomal locus. In non-limiting examples, the analysis comprises determining the identity of a polymorphic nucleotide in at least one polymorphic site within the 2P15 chromosome locus having a reference sequence number rs10865331 on chromosome 2. Suitably, the presence of A (adenine) at rs10865331, indicates that the subject has AS or is at risk of developing AS.

In some embodiments, the sample is further analyzed for the presence of a polymorphism in the 21Q22 chromosomal locus. In illustrative examples, the analysis comprises determining the identity of a polymorphic nucleotide in at least one polymorphic site within the 21Q22 chromosome locus having reference sequence number rs2242944 on chromosome 21. Suitably, the presence of G at rs2242944, indicates that the subject has AS or is at risk of developing AS.

In some embodiments, the sample is further analyzed for the presence of a polymorphism in the HLA-B27 gene.

In certain embodiments, the methods for diagnosing the presence or risk of development of AS comprise (a) obtaining from the subject a biological sample comprising at least a portion of an AS marker selected from an ARTS-1 gene, an IL-23R gene, a TNFR1 gene locus, a TRADD gene locus and a 2P15 chromosome locus or an expression product thereof; and (b) analyzing the sample for a polymorphism in the AS marker, which is indicative of the presence or risk of development of AS, wherein the polymorphism is selected from:

(a) a polymorphism in the ARTS-1 gene selected from a G (guanine) at reference sequence (rs) 27044; a T (thymine) at rs30187; or a C (cytosine) at rs17482078;

(b) a polymorphism in the IL-23R gene selected from a T (thymine) at rs10489629, a G (guanine) at rs1343151, or an A (adenine) at rs10889677 or rs11209032;

(c) a polymorphism in the TNFR1 gene locus, represented by a C (cytosine) at rs4149576;

(d) a polymorphism in the TRADD gene locus, represented by a G (guanine) at rs9033; and

(e) a polymorphism in the 21Q22 chromosome locus, represented by a G (guanine) at rs2242944.

In some embodiments, the presence of a G at rs27044 is indicated by detecting the presence of a glutamine (Gln) at residue 730 of the ARTS-1 polypeptide. Suitably, the presence of a C at rs17482078 is indicated by detecting the presence of an arginine (Arg) at residue 725 of the ARTS-1 polypeptide. In some embodiments, the presence of a T at rs30187 is indicated by detecting the presence of a lysine (Lys) at residue 528 of the ARTS-1 polypeptide.

Suitably, the sample is further analyzed for the presence of at least one other AS-associated polymorphism. For example, the sample may be further analyzed for the presence of at least one other ARTS-1 polymorphism selected from a T (thymine) at rs2287987; or a C (cytosine) at rs10050860. In illustrative examples of this type, the presence of a T at rs2287987 is indicated by detecting the presence of a methionine (Met) at residue 349 of the ARTS-1 polypeptide; or the presence of C at rs10050860 is indicated by detecting the presence of an aspartic acid (Asp) at residue 575 of the ARTS-1 polypeptide.

Alternatively, or in addition, the sample is further analyzed for the presence of at least one other IL-23R polymorphism selected from a T (thymine) at rs11465804, a G (guanine) at rs11209026, a C (cytosine) at rs1495965; or an A (adenine) at rs1004819. In illustrative examples of this type, the presence of a G at rs11209026 is indicated by detecting the presence of an Arg at residue 381 of the IL-23R polypeptide

If desired, the sample may be further analyzed for the presence of a polymorphism in the 21Q22 chromosome locus, represented by a G at rs2242944.

In some embodiments, the sample is analyzed for the presence of at least two AS markers, illustrative combinations of which include (1) a polymorphism in the TNFR1 gene locus and a polymorphism in the chromosome locus 2P15, (2) a polymorphism in the TNFR1 gene locus and a polymorphism in the chromosome locus 21Q22, (3) a polymorphism in the TNFR1 gene locus and a polymorphism in the TRADD gene locus, (4) a polymorphism in the TNFR1 gene locus and a polymorphism in the ARTS-1 gene, (5) a polymorphism in the TNFR1 gene locus and a polymorphism in the IL-23R gene, (6) a polymorphism in the chromosome locus 2P15 and a polymorphism in the chromosome locus 21Q22, (7) a polymorphism in the chromosome locus 2P15 and a polymorphism in the TRADD gene locus, (8) a polymorphism in the chromosome locus 21Q22 and a polymorphism in the TRADD gene locus, (9) a polymorphism in the TRADD gene locus and a polymorphism in the ARTS-1 gene, (10) a polymorphism in the TRADD gene locus and a polymorphism in the IL-23R gene, (11) a polymorphism in the chromosome locus 2P15 and a polymorphism in the ARTS-1 gene, (12) a polymorphism in the chromosome locus 2P15 and a polymorphism in the IL-23R gene, (13) a polymorphism in the chromosome locus 21Q22 and a polymorphism in the ARTS-1 gene, (14) a polymorphism in the chromosome locus 21Q22 and a polymorphism in the IL-23R gene, (15) a polymorphism in the ARTS-1 gene and a polymorphism in the IL-23R gene, (16) a polymorphism in the TNFR1 gene locus and a polymorphism in the chromosome locus 2P15 and a polymorphism in the chromosome locus 21Q22, (17) a polymorphism in the TNFR1 gene locus and a polymorphism in the chromosome locus 2P15 and a polymorphism in the TRADD gene locus, (18) a polymorphism in the TNFR1 gene locus and a polymorphism in the chromosome locus 21Q22 and a polymorphism in the TRADD gene locus, (19) a polymorphism in the chromosome locus 21Q22 and a polymorphism in the chromosome locus 2P15 and a polymorphism in the TRADD gene locus, (20) a polymorphism in the ARTS-1 gene and a polymorphism in the chromosome locus 2P15 and a polymorphism in the chromosome locus 21Q22, (21) a polymorphism in the ARTS-1 gene and a polymorphism in the chromosome locus 2P15 and a polymorphism in the TRADD gene locus, (22) a polymorphism in the ARTS-1 gene and a polymorphism in the chromosome locus 2P15 and a polymorphism in the TNFR1 gene locus, (23) a polymorphism in the ARTS-1 gene and a polymorphism in the chromosome locus 21Q22 and a polymorphism in the TRADD gene locus, (24) a polymorphism in the ARTS-1 gene and a polymorphism in the chromosome locus 21Q22 and a polymorphism in the TNFR1 gene locus, (25) a polymorphism in the ARTS-1 gene and a polymorphism in the TNFR1 gene locus and a polymorphism in the TRADD gene locus, (26) a polymorphism in the ARTS-1 gene and a polymorphism in the IL-23R gene and a polymorphism in the chromosome locus 2P15, (27) a polymorphism in the ARTS-1 gene and a polymorphism in the IL-23R gene and a polymorphism in the chromosome locus 2P1Q22, (28) a polymorphism in the ARTS-1 gene and a polymorphism in the IL-23R gene and a polymorphism in the TRADD gene locus, and (29) a polymorphism in the ARTS-1 gene and a polymorphism in the IL-23R gene and a polymorphism in the TNFR1 gene locus. In still other embodiments, the sample is analyzed for the presence a polymorphism in four or each of the ARTS-1 gene, the TNFR1 gene locus, the TRADD gene locus, the IL23R gene and the chromosomal loci 2P15 and 21Q22, as broadly described above.

The polymorphism can be detected by any method known in the art including, but not limited to: polymerase chain reaction (PCR), ligase chain reaction (LCR), hybridization analysis, digestion with nucleases, restriction fragment length polymorphism, antibody detection methods, direct sequencing or any combination thereof.

In certain embodiments, the presence or risk of development of AS in a the subject is determined from the subject's AS marker genotype. A subject who has at least one polymorphism statistically associated with AS possesses a factor contributing to an increased risk of AS, as compared to a subject without the polymorphism.

In another aspect, the present invention contemplates the use of a nucleic acid construct comprising at least a portion of an AS marker as broadly described above, which contains at least one AS-associated polymorphism, for diagnosing the presence or risk of development of AS. In some embodiments, the at least a portion of the AS marker is operably connected to a regulatory element, which is operable in a host cell. In certain embodiments, the construct is in the form of a vector, especially an expression vector. In illustrative examples of this type, the vector is used as a positive control.

In yet another aspect, the present invention contemplates the use of isolated host cells containing a nucleic acid construct or vector as broadly described above for diagnosing the presence or risk of development of AS. In certain embodiments, the host cells are selected from bacterial cells, yeast cells and insect cells. In illustrative examples of this type, the host cells are used in the production of at least one polypeptide selected from IL-1R1, IL-1R2, CD74, TCOF1, UBE2E3, ARTS-1 and IL-23R polypeptides for use as a positive control. In some embodiments, the polypeptide(s) may be fragmented and analysed using mass spectrometry techniques.

Still another aspect of the present invention relates to the use of one or more oligonucleotides that hybridize to at least one AS-associated polymorphic site in an AS marker as broadly described above in the manufacture of a kit for detecting the presence or diagnosing the risk of development of AS. The kit may comprise one or more oligonucleotides capable of detecting a polymorphism in an AS marker of the invention as well as instructions for using the kit to detect AS or to diagnose the risk of developing AS. In some embodiments, the oligonucleotides each comprise a sequence that hybridizes under stringent hybridization conditions to at least one AS-associated polymorphism in any one or more of the AS markers as broadly described above. In some embodiments, the oligonucleotides each comprise a sequence that is fully complementary to a nucleic acid sequence comprising an AS-associated polymorphism.

Yet another aspect of the present invention relates to the use of at least a portion of an AS marker polypeptide encoded by an AS marker gene selected from the IL-1R1 gene, the IL-1R2 gene, the CD74 gene or a gene genetically linked thereto (e.g., TCOF1), the chromosome locus 2Q31.3 or a gene genetically linked thereto (e.g., UBE2E3), the ARTS-1 gene and the IL-23R gene, which comprises at least one AS-associated polymorphic site, or a construct comprising a nucleic acid sequence that encodes the at least a portion of the AS marker polypeptide, or an antigen-binding molecule that is immuno-interactive with an AS-associated polymorphism of the present invention, in the manufacture of a kit, for diagnosing the presence or risk of development of AS in a subject. In illustrative embodiments, the at least a portion of the AS marker polypeptide and/or the constructs are used as positive controls in the diagnostic methods of the invention and the antigen-binding molecule is used to specifically recognize and detect an individual polymorphism of the present invention.

The invention further provides methods for treating AS in a subject. These methods generally comprise analysing a biological sample obtained from the subject for the presence of at least one AS-associated polymorphism in an AS marker as broadly described above and exposing the subject to a treatment that ameliorates or reverses the symptoms of AS on the basis that the subject tests positive for the polymorphism(s).

### BRIEF DESCRIPTION OF THE DRAWINGS

FIG. 1 is a graphical representation showing post-test probability of AS given test results, comparing B27 tests and other combinations of genetic markers.

FIG. 2 shows a portion of the genomic sequence comprising the polymorphism rs949963 (chr 2: 102,136,017-102,136,718). The rs949963 polymorphism is at position 102,136,218 on chromosome 2 within the IL-1R1 gene locus, which spans positions 102,136,834-102,162,766 on chromosome 2.

FIG. 3 shows a portion of the genomic sequence comprising the polymorphism rs2310173 (chr 2: 102,029,560-102,030,511). The rs2310173 polymorphism is at position 102,030,060 on chromosome 2 within the IL-1R2 gene locus, which spans positions 101,974,738-102,011,312 on chromosome 2.

FIG. 4 shows a portion of the genomic sequence comprising the polymorphism rs15251 (chr 5: 149,756,024-149,756,825). The rs15251 polymorphism is at position 149,756,425 on chromosome 5 within the Treacher Collins-Franceschetti syndrome 1 (TCOF1) gene, which spans positions 149,717,428-149,760,063 on chromosome 5, and which is considered to be in genetic linkage with the CD74 gene, spanning positions 149,761,399-149,772,525 on chromosome 5.

FIG. 5 shows a portion of the genomic sequence comprising the polymorphism rs1018326 (chr 2: 181,715,744-181,716,345). The rs1018326 polymorphism is at position 181,716,045 on chromosome 2 within the 2Q31.3 locus, which spans positions 180,400,000-182,700,000 on chromosome 2, and which is considered to be in genetic linkage with the ubiquitin-conjugating enzyme E2 E3 (UBE2E3) gene located at positions 181,553,357-181,636,397 on chromosome 2.

FIG. 6 shows a portion of the genomic sequence comprising the polymorphism rs10517820 (chr 4: 62,963,226-62,963,427). The rs1018326 polymorphism is at position 62,963,327 on chromosome 4 within the 4Q13.1 locus, spanning positions 59,200,000-63,300,000 on chromosome 4.

FIG. 7 shows a portion of the genomic sequence comprising the polymorphism rs27044 (chr 5: 96144307-96144908). The rs27044 polymorphism is at position 96,144,608 on chromosome 5 within the ARTS-1 gene.

FIG. 8 shows a portion of the genomic sequence comprising the polymorphism rs17482078 (chr 5: 96144321-96144922). The rs17482078 polymorphism is at position 96,144,622 on chromosome 5 within the ARTS-1 gene.

FIG. 9 shows a portion of the genomic sequence comprising the polymorphism rs30187 (chr 5: 96149785-96150386). The rs30187 polymorphism is at position 96,150,086 on chromosome 5 within the ARTS-1 gene.

FIG. 10 shows a portion of the genomic sequence comprising the polymorphism rs10489629 (chr 1: 67460937-67460937). The rs10489629 polymorphism is at position 67,460,937 on chromosome 1 within the IL-23R gene.

FIG. 11 shows a portion of the genomic sequence comprising the polymorphism rs1343151 (chr 1: 67491416-67492017). The rs1343151 polymorphism is at position 67,491,717 on chromosome 1 within the IL-23R gene.

FIG. 12 shows a portion of the genomic sequence comprising the polymorphism rs10889677 (chr 1: 67497407-67498008). The rs10889677 polymorphism is at position 67,497,708 on chromosome 1 within the IL-23R gene.

FIG. 13 shows a portion of the genomic sequence comprising the polymorphism rs11209032 (chr 1: 67,512,379-67,512,980). The rs11209032 polymorphism is at position 67,512,680 on chromosome 1 within the IL-23R gene.

FIG. 14 shows a portion of the genomic sequence comprising the polymorphism rs4149576 (chr 12: 6319075-6319676). The rs4149576 polymorphism is at position 6,319,376 on chromosome 12 within the TNFR1 gene locus.

FIG. 15 shows a portion of the genomic sequence comprising the polymorphism rs9033 (chr 16: 65,739,199-65,739,799). The rs9033 polymorphism is at position 65,739,500 on chromosome 16 within the TRADD gene locus.

FIG. 16 shows a portion of the genomic sequence comprising the polymorphism rs2242944 (chr 21: 39,386,547-39,387,548). The rs2242944 polymorphism is at position 39,387,048 on chromosome 21 within the 21Q22 chromosome locus.

FIG. 17 shows a portion of the genomic sequence comprising the polymorphism rs10050860 (chr 5: 96,147,665-96,148,266). The rs10050860 polymorphism is at position 96,147,966 on chromosome 5 within the ARTS-1 gene.

FIG. 18 shows a portion of the genomic sequence comprising the polymorphism rs2287987 (chr 5: 96,154,990-96,155,591). The rs2287987 polymorphism is at position 96,155,291 on chromosome 5 within the ARTS-1 gene.

FIG. 19 shows a portion of the genomic sequence comprising the polymorphism rs1004819 (chr 1: 67,442,312-67,443,004). The rs1004819 polymorphism is at position 67,442,801 on chromosome 1 within the IL-23R gene.

FIG. 20 shows a portion of the genomic sequence comprising the polymorphism rs11465804 (chr 14: 67,475,013-67,475,214). The rs11465804 polymorphism is at position 67,475,114 on chromosome 1 within the IL-23R gene.

FIG. 21 shows a portion of the genomic sequence comprising the polymorphism rs11209026 (chr 1: 67,478,245-67,478,846). The rs11209026 polymorphism is at position 67,478,546 on chromosome 1 within the IL-23R gene.

FIG. 22 shows a portion of the genomic sequence comprising the polymorphism rs1495965 (chr 1: 67,525,837-67,526,485). The rs1495965 polymorphism is at position 67,526,096 on chromosome 1 within the IL-23R gene.

FIG. 23 shows a portion of the genomic sequence comprising the polymorphism rs10865331 (chr 2: 62,404,333-62,405,817). The rs10865331 polymorphism is at position 62,404,976 on chromosome 2 within the 2P15 chromosome locus.

FIG. 24 is a graphical representation showing post-test probability of AS given test results, comparing B27 tests and other combinations of genetic markers.

FIG. 25 is a graphical representation showing post-probability of AS given test results, comparing MRI scanning with genetic tests.

FIG. 26 is a graphical representation of minus log₁₀ p values for the Cochrane-Armitage test of trend for genome-wide association scans of ankylosing spondylitis (AS). The spacing between SNPs on the plot is uniform and does not reflect distances between the SNPs. The vertical dashed lines reflect chromosomal boundaries. The horizontal dashed lines display the cutoff for p=0.05 after Bonferroni correction.

FIG. 27 is a graphical representation of minus log₁₀ p values for the Cochrane-Armitage test of trend for genome-wide association scans involving combined controls. The spacing between SNPs on the plot is uniform and does not reflect distances between the SNPs. The vertical dashed lines reflect chromosomal boundaries. The horizontal dashed lines display the cutoff for p=0.05 after Bonferroni correction.

FIG. 28 is a graphical representation of Cochrane-Armitage significance tests after each stage of genotype filtering for Ankylosing Spondylitis. The filters employed are Stage 1: no SNPs removed from analyses; Stage 2: SNPs with >10% missing genotypes removed from analyses; Stage 3: SNPs failing Hardy-Weinberg at p<10⁻⁷ in control individuals removed; Stage 4: SNPs that differ in missing rate between cases and controls at p<10-4 removed from analyses and; Stage 5: Upon manual inspection of the raw genotype intensities, SNPs that poorly cluster removed from subsequent analyses.

TABLE A
BRIEF DESCRIPTION OF THE SEQUENCES

| SEQUENCE | | |
|---|---|---|
| ID NUMBER | SEQUENCE | LENGTH |
| SEQ ID NO: 1 | Nucleotide sequence corresponding to a portion of the | 701 nts |
| | genomic sequence of human chromosome 2, comprising | |
| | the polymorphism rs949963 (chr 2: 102,136,017- | |
| | 102,136,718) within the IL-1R1 gene locus | |
| SEQ ID NO: 2 | Nucleotide sequence corresponding to a portion of the | 951 nts |
| | genomic sequence of human chromosome 2, comprising | |
| | the polymorphism rs2310173 (chr 2: 102,029,560- | |
| | 102,030,511) within the IL-1R2 gene locus | |
| SEQ ID NO: 3 | Nucleotide sequence corresponding to a portion of the | 801 nts |
| | genomic sequence of human chromosome 5, comprising | |
| | the polymorphism rs15251 (chr 5: 149,756,024- | |
| | 149,756,825) within the TCOF1 gene that is genetically | |
| | linked to the CD74 gene locus | |
| SEQ ID NO: 4 | Nucleotide sequence corresponding to a portion of the | 601 nts |
| | genomic sequence of human chromosome 2, comprising | |
| | the polymorphism rs1018326 (chr 2: 181,715,744- | |
| | 181,716,345) in the UBE2E3 gene that is in genetic linkage | |
| | with the 2Q31.3 chromosome locus | |
| SEQ ID NO: 5 | Nucleotide sequence corresponding to a portion of the | 201 nts |
| | genomic sequence of human chromosome 4, comprising | |
| | the polymorphism rs10517820 (chr 4: 62,963,226- | |
| | 62,963,427) within the 4Q13.1 chromosome locus | |
| SEQ ID NO: 6 | Nucleotide sequence corresponding to a portion of the | 601 nts |
| | genomic sequence of human chromosome 5, comprising | |
| | the polymorphism rs27044 (chr 5: 96144307-96144908) | |
| | within the ARTS-1 gene | |
| SEQ ID NO: 7 | Nucleotide sequence corresponding to a portion of the | 601 nts |
| | genomic sequence of human chromosome 5, comprising | |
| | the polymorphism rs17482078 (chr 5: 96144321- | |
| | 96144922) within the ARTS-1 gene | |
| SEQ ID NO: 8 | Nucleotide sequence corresponding to a portion of the | 601 nts |
| | genomic sequence of human chromosome 5, comprising | |
| | the polymorphism rs30187 (chr 5: 96149785-96150386) | |
| | within the ARTS-1 gene | |
| SEQ ID NO: 9 | Nucleotide sequence corresponding to a portion of the | 201 nts |
| | genomic sequence of human chromosome 1, comprising | |
| | the polymorphism rs10489629 (chr 1: 67460937- | |
| | 67460937) within the IL-23R gene | |
| SEQ ID NO: 10 | Nucleotide sequence corresponding to a portion of the | 601 nts |
| | genomic sequence of human chromosome 1, comprising | |
| | the polymorphism rs1343151 (chr 1: 67491416-67492017) | |
| | within the IL-23R gene | |
| SEQ ID NO: 11 | Nucleotide sequence corresponding to a portion of the | 601 nts |
| | genomic sequence of human chromosome 1, comprising | |
| | the polymorphism rs10889677 (chr 1: 67497407- | |
| | 67498008) within the IL-23R gene | |
| SEQ ID NO: 12 | Nucleotide sequence corresponding to a portion of the | 601 nts |
| | genomic sequence of human chromosome 1, comprising | |
| | the polymorphism rs11209032 (chr 1: 67,512,379- | |
| | 67,512,980) within the IL-23R gene | |
| SEQ ID NO: 13 | Nucleotide sequence corresponding to a portion of the | 601 nts |
| | genomic sequence of human chromosome 12, comprising | |
| | the polymorphism rs4149576 (chr 12: 6319075-6319676) | |
| | within the TNFR1 gene locus | |
| SEQ ID NO: 14 | Nucleotide sequence corresponding to a portion of the | 600 nts |
| | genomic sequence of human chromosome 16, comprising | |
| | the polymorphism rs9033 (chr 16: 65,739,199-65,739,799) | |
| | within the TRADDgene locus | |
| SEQ ID NO: 15 | Nucleotide sequence corresponding to a portion of the | 1001 nts |
| | genomic sequence of human chromosome 21, comprising | |
| | the polymorphism rs2242944 (chr 21: 39,386,547- | |
| | 39,387,548) within the 21Q22 chromosome locus | |
| SEQ ID NO: 16 | Nucleotide sequence corresponding to a portion of the | 601 nts |
| | genomic sequence of human chromosome 5, comprising | |
| | the polymorphism rs10050860 (chr 5: 96,147,665- | |
| | 96,148,266) within the ARTS-1 gene | |
| SEQ ID NO: 17 | Nucleotide sequence corresponding to a portion of the | 601 nts |
| | genomic sequence of human chromosome 5, comprising | |
| | the polymorphism rs2287987 (chr 5: 96,154,990- | |
| | 96,155,591) within the ARTS-1 gene | |
| SEQ ID NO: 18 | Nucleotide sequence corresponding to a portion of the | 692 nts |
| | genomic sequence of human chromosome 1, comprising | |
| | the polymorphism rs1004819 (chr 1: 67,442,312- | |
| | 67,443,004) within the IL-23R gene | |
| SEQ ID NO: 19 | Nucleotide sequence corresponding to a portion of the | 201 nts |
| | genomic sequence of human chromosome 1, comprising | |
| | the polymorphism rs11465804 (chr 14: 67,475,013- | |
| | 67,475,214) within the IL-23R gene | |
| SEQ ID NO: 20 | Nucleotide sequence corresponding to a portion of the | 601 nts |
| | genomic sequence of human chromosome 1, comprising | |
| | the polymorphism rs11209026 (chr 1: 67,478,245- | |
| | 67,478,846) within the IL-23R gene | |
| SEQ ID NO: 21 | Nucleotide sequence corresponding to a portion of the | 648 nts |
| | genomic sequence of human chromosome 1, comprising | |
| | the polymorphism rs1495965 (chr 1: 67,525,837- | |
| | 67,526,485) within the IL-23R gene | |
| SEQ ID NO: 22 | Nucleotide sequence corresponding to a portion of the | 1484 nts |
| | genomic sequence of human chromosome 2, comprising | |
| | the polymorphism rs10865331 (chr 2: 62,404,333- | |
| | 62,405,817) within the 2P15 chromosome locus | |

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

“Allele” is used herein to refer to a variant of a gene found at the same place or locus of a chromosome.

“Amplification product” refers to a nucleic acid product generated by nucleic acid amplification techniques.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity.

The term “biological sample” as used herein refers to a sample that may be extracted, untreated, treated, diluted or concentrated from a patient. Suitably, the biological sample is selected from any part of a patient's body, including, but lot limited to hair, skin, nails, tissues or bodily fluids such as saliva and blood.

Throughout this specification, unless the context requires otherwise, the words “comprise,” “comprises” and “comprising” will be understood to imply the inclusion of a stated step or element or group of steps or elements but not the exclusion of any other step or element or group of steps or elements.

By “corresponds to” or “corresponding to” is meant (a) a polynucleotide having a nucleotide sequence that is substantially identical or complementary (e.g., at least 75%, 76%, 77%, 78%, 79%, 80%, 81%, 82%, 83%, 84%, 85%, 86%, 97%, 88%, 89%, 90%, 91%, 92%, 93%, 94%, 95%, 96%, 97%, 98% or 99% to all or a portion of a reference polynucleotide sequence.

By “effective amount”, in the context of treating or preventing a condition is meant the administration of that amount of active to an individual in need of such treatment or prophylaxis, either in a single dose or as part of a series, that is effective for treatment of, or prophylaxis against, that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

As used herein, the terms “function” and “functional” and the like refer to a biological, enzymatic, or therapeutic function.

By “gene” is meant a unit of inheritance that occupies a specific locus on a chromosome and consists of transcriptional and/or translational regulatory sequences and/or a coding region and/or non-translated sequences (i.e., introns, 5′ and 3′ untranslated sequences).

“Genetic linkage” refers to an association of two or more non-allelic genetic loci which do not show independent assortment, often due to physical association on the same chromosome.

“Homology” refers to the percentage number of nucleic or amino acids that are identical or constitute conservative substitutions. Homology may be determined using sequence comparison programs such as GAP (Deveraux et al., 1984, Nucleic Acids Research 12, 387-395) which is incorporated herein by reference. In this way sequences of a similar or substantially different length to those cited herein could be compared by insertion of gaps into the alignment, such gaps being determined, for example, by the comparison algorithm used by GAP.

The term “host cell” includes an individual cell or cell culture which can be or has been a recipient of any recombinant vector(s) or isolated polynucleotide of the invention. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with a recombinant vector or a polynucleotide of the invention. A host cell which comprises a recombinant vector of the invention is a “recombinant host cell”.

“Hybridization” is used herein to denote the pairing of complementary nucleotide sequences to produce a DNA-DNA hybrid or a DNA-RNA hybrid. Complementary base sequences are those sequences that are related by the base-pairing rules. In DNA, A pairs with T and C pairs with G. In RNA U pairs with A and C pairs with G. In this regard, the terms “match” and “mismatch” as used herein refer to the hybridization potential of paired nucleotides in complementary nucleic acid strands. Matched nucleotides hybridise efficiently, such as the classical A-T and G-C base pair mentioned above. Mismatches are other combinations of nucleotides that do not hybridise efficiently.

Reference herein to “immuno-interactive” includes reference to any interaction, reaction, or other form of association between molecules and in particular where one of the molecules is, or mimics, a component of the immune system.

By “isolated” is meant material that is substantially or essentially free from components that normally accompany it in its native state.

The term “locus,” or “genetic locus” generally refers to a genetically defined region of a chromosome carrying a gene or any other characterized sequence. In some embodiments, the locus is genetically linked to a polymorphic site as defined herein.

The term “marker”, as used herein generally refers to a genetic locus, including a gene or other characterized sequence, which is genetically linked to a trait or phenotype of interest. The term “genetically linked” as used herein refers to two or more loci that are predictably inherited together during random crossing or intercrossing.

By “obtained from” is meant that a sample such as, for example, a polynucleotide extract or polypeptide extract is isolated from, or derived from, a particular source of the subject. For example, the extract can be obtained from a tissue or a biological fluid isolated directly from the subject.

The term “oligonucleotide” as used herein refers to a polymer composed of a multiplicity of nucleotide residues (deoxyribonucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotide residues and linkages between them are naturally occurring, it will be understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule can vary depending on the particular application. An oligonucleotide is typically rather short in length, generally from about 10 to 30 nucleotide residues, but the term can refer to molecules of any length, although the term “polynucleotide” or “nucleic acid” is typically used for large oligonucleotides.

The terms “patient” and “subject” are used interchangeably and refer to patients and subjects of human or other mammal and includes any individual it is desired to examine or treat using the methods of the invention. However, it will be understood that “patient” does not imply that symptoms are present. Suitable mammals that fall within the scope of the invention include, but are not restricted to, primates, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes).

By “pharmaceutically acceptable carrier” is meant a solid or liquid filler, diluent or encapsulating substance that can be safely used in topical or systemic administration to a animal, preferably a mammal including humans.

The term “polymorphism”, as used herein, refers to a difference in the nucleotide or amino acid sequence of a given region as compared to a nucleotide or amino acid sequence in a homologous-region of another individual, in particular, a difference in the nucleotide of amino acid sequence of a given region which differs between individuals of the same species. A polymorphism is generally defined in relation to a reference sequence. Polymorphisms include single nucleotide differences, differences in sequence of more than one nucleotide, and single or multiple nucleotide insertions, inversions and deletions; as well as single amino acid differences, differences in sequence of more than one amino acid, and single or multiple amino acid insertions, inversions, and deletions. A “polymorphic site” is the locus at which the variation occurs. It shall be understood that where a polymorphism is present in a nucleic acid sequence, and reference is made to the presence of a particular base or bases at a polymorphic site, the present invention encompasses the complementary base or bases on the complementary strand at that site.

The term “polynucleotide” or “nucleic acid” as used herein designates mRNA, RNA, cRNA, cDNA or DNA. The term typically refers to oligonucleotides greater than 30 nucleotide residues in length.

“Polypeptide”, “peptide” and “protein” are used interchangeably herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers.

By “primer” is meant an oligonucleotide which, when paired with a strand of DNA, is capable of initiating the synthesis of a primer extension product in the presence of a suitable polymerizing agent. The primer is preferably single-stranded for maximum efficiency in amplification but can alternatively be double-stranded. A primer must be sufficiently long to prime the synthesis of extension products in the presence of the polymerization agent. The length of the primer depends on many factors, including application, temperature to be employed, template reaction conditions, other reagents, and source of primers. For example, depending on the complexity of the target sequence, the oligonucleotide primer typically contains 15 to 35 or more nucleotide residues, although it can contain fewer nucleotide residues. Primers can be large polynucleotides, such as from about 200 nucleotide residues to several kilobases or more. Primers can be selected to be “substantially complementary” to the sequence on the template to which it is designed to hybridize and serve as a site for the initiation of synthesis. By “substantially complementary”, it is meant that the primer is sufficiently complementary to hybridize with a target polynucleotide. Preferably, the primer contains no mismatches with the template to which it is designed to hybridize but this is not essential. For example, non-complementary nucleotide residues can be attached to the 5′ end of the primer, with the remainder of the primer sequence being complementary to the template. Alternatively, non-complementary nucleotide residues or a stretch of non-complementary nucleotide residues can be interspersed into a primer, provided that the primer sequence has sufficient complementarity with the sequence of the template to hybridize therewith and thereby form a template for synthesis of the extension product of the primer.

“Probe” refers to a molecule that binds to a specific sequence or sub-sequence or other moiety of another molecule. Unless otherwise indicated, the term “probe” typically refers to a polynucleotide probe that binds to another polynucleotide, often called the “target polynucleotide”, through complementary base pairing. Probes can bind target polynucleotides lacking complete sequence complementarity with the probe, depending on the stringency of the hybridization conditions. Probes can be labeled directly or indirectly.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Iie, Phe, Tyr, Trp, Lys, Arg, H is, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

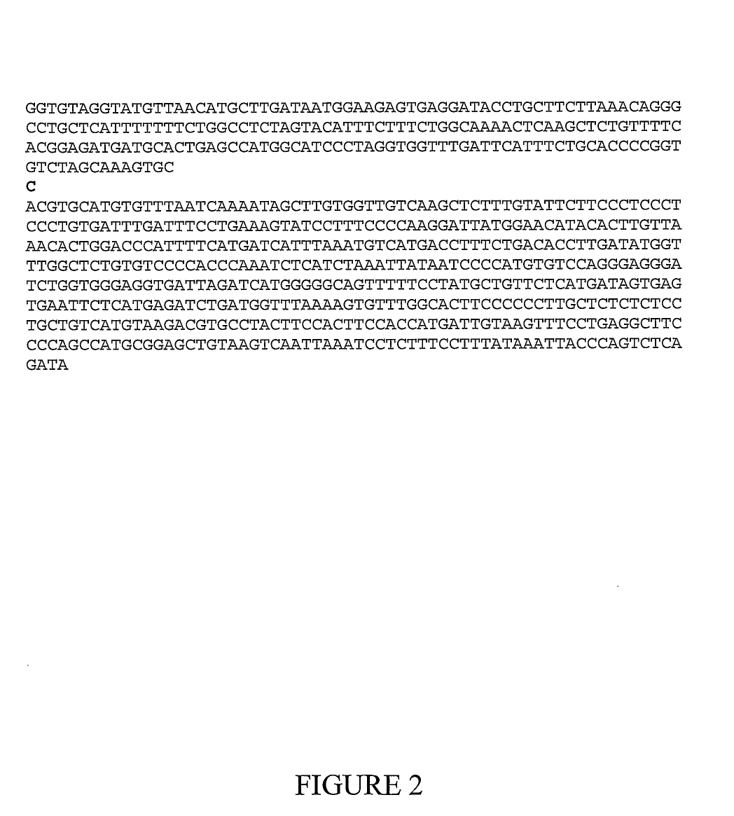

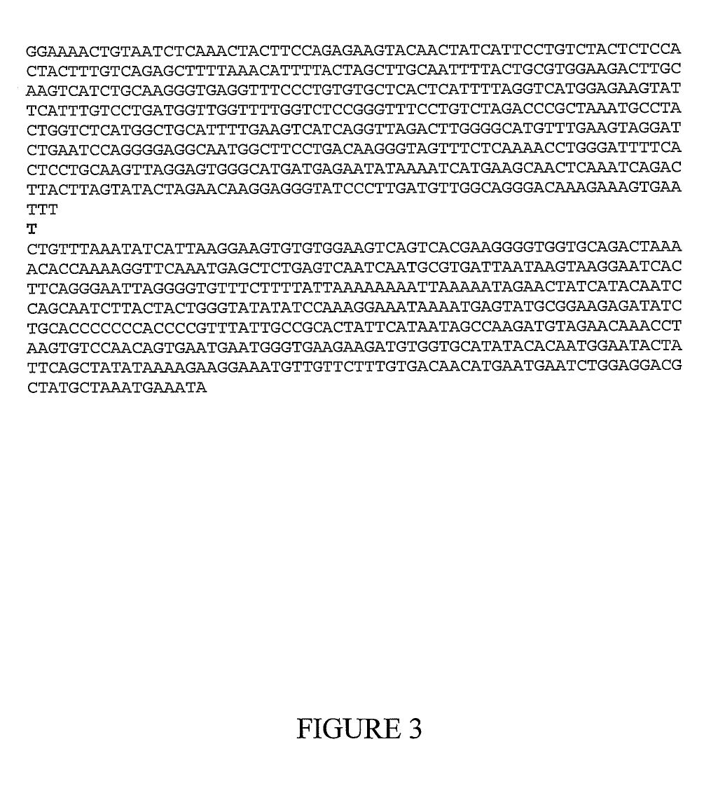

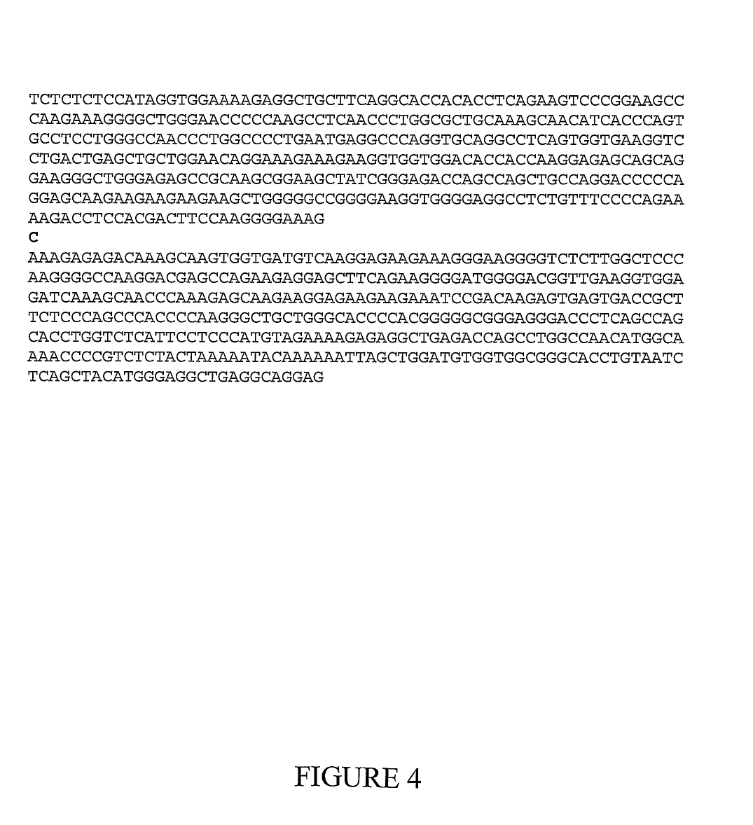

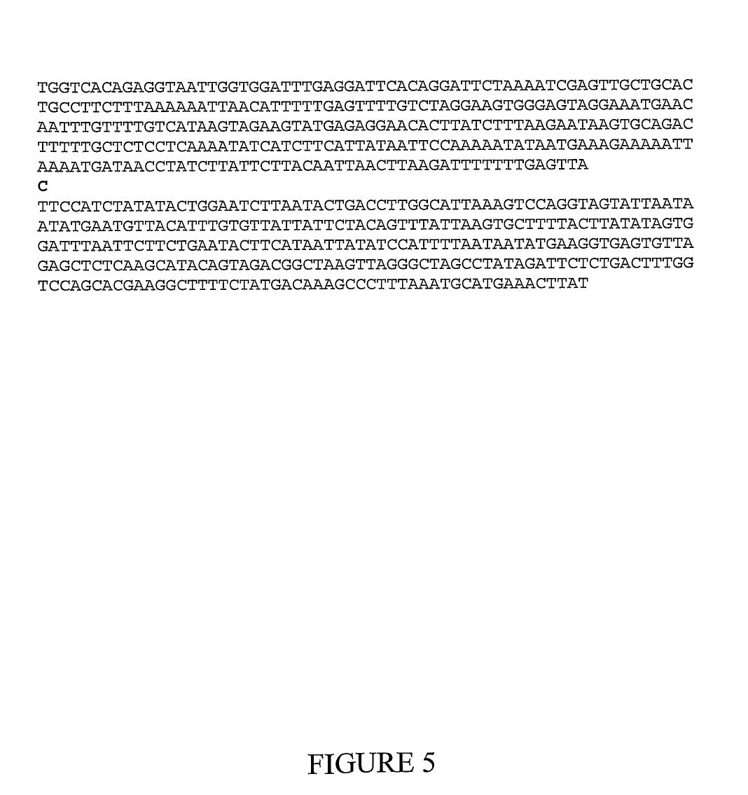

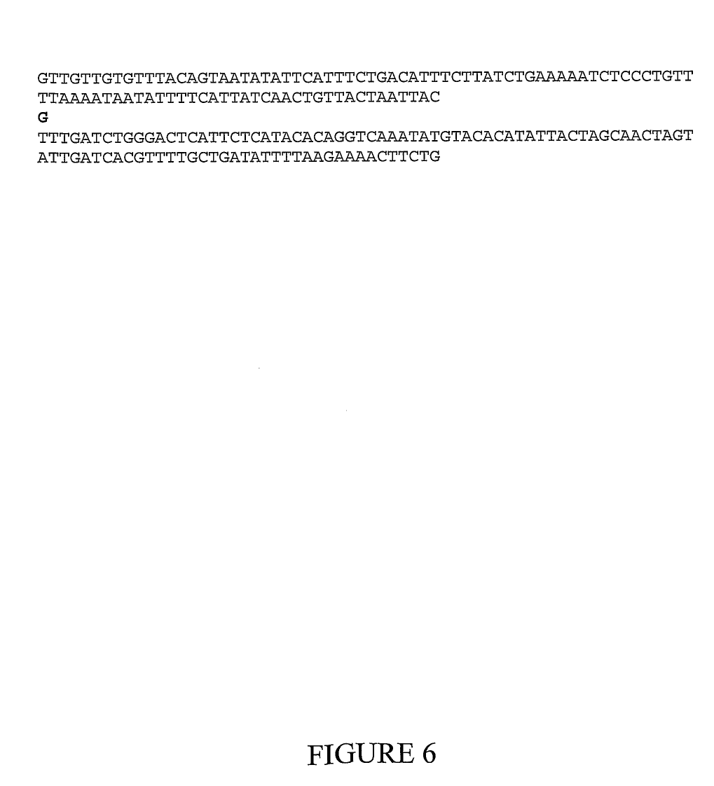

“Single nucleotide polymorphism (SNP)” as used herein refers to a change in which a single base in the DNA differs (such as via substitutions, addition or deletion) from the usual base at that position. For example, a single nucleotide polymorphism is characterized by the presence in a population of one or two, three or four nucleotides (i.e., adenosine, cytosine, guanosine or thymidine) at a particular locus in a genome such as the human genome. It will be recognized that while the methods of the present invention are directed to the identification of certain SNPs within the IL-1R1 gene, the IL-1R2 gene, the CD74 gene locus, the 2Q31.3 chromosome locus and the 4Q13.1 chromosomal locus (e.g., FIGS. 2-6), the methods can be used to identify other AS-associated SNPs either alone or in combination with the exemplified SNPs, or combined with methods for determining other AS-associated polymorphisms in the IL-1R1 gene, the IL-1R2 gene, the CD74 gene locus and the 2Q31.3 chromosome locus and the 4Q13.1 chromosomal locus sequences, to increase the accuracy of the determination.

“Stringency” as used herein, refers to the temperature and ionic strength conditions, and presence or absence of certain organic solvents, during hybridization and washing procedures. The higher the stringency, the higher will be the degree of complementarity between immobilized target nucleotide sequences and the labeled probe polynucleotide sequences that remain hybridized to the target after washing. The term “high stringency” refers to temperature and ionic conditions under which only nucleotide sequences having a high frequency of complementary bases will hybridize. The stringency required is nucleotide sequence dependent and depends upon the various components present during hybridization. Generally, stringent conditions are selected to be about 10 to 20° C. lower than the thermal melting point (Tₘ) for the specific sequence at a defined ionic strength and pH. The Tₘ is the temperature (under defined ionic strength and pH) at which 50% of a target sequence hybridizes to a complementary probe.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

By “vector” is meant a polynucleotide molecule, preferably a DNA molecule derived, for example, from a plasmid, bacteriophage, yeast or virus, into which a polynucleotide can be inserted or cloned. A vector preferably contains one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, i.e., a vector that exists as an extra-chromosomal entity, the replication of which is independent of chromosomal replication, e.g., a linear or closed circular plasmid, an extra-chromosomal element, a mini-chromosome, or an artificial chromosome. The vector can contain any means for assuring self-replication. Alternatively, the vector can be one which, when introduced into the host cell, is integrated into the genome and replicated together with the chromosome(s) into which it has been integrated. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. In the present case, the vector is preferably a viral or viral-derived vector, which is operably functional in animal and preferably mammalian cells. Such vector may be derived from a poxvirus, an adenovirus or yeast. The vector can also include a selection marker such as an antibiotic resistance gene that can be used for selection of suitable transformants. Examples of such resistance genes are known to those of skill in the art and include the nptII gene that confers resistance to the antibiotics kanamycin and G418 (Geneticin®) and the hph gene which confers resistance to the antibiotic hygromycin B.

### 2. Polymorphism of the Invention

The present invention is based in part on the determination that (1) polymorphisms within the IL-1R1 gene locus, the IL-1R2 gene locus, the CD74 gene locus and the chromosome loci 2Q31.3 and 4Q13.1, and (2) certain polymorphisms within the ARTS-1 and IL-23R genes, the TNFR1 and TRADD gene loci and chromosome loci 2P15 and 21Q22 (also referred to herein as AS makers) are associated with the presence or risk of developing AS. Accordingly, the present invention provides methods for diagnosing the presence or risk of development of AS in a subject, wherein the methods comprise (a) obtaining from the subject a biological sample comprising at least a portion of an AS marker selected from (1) an IL-1R1 gene locus or an expression product thereof, (2) an IL-1R2 gene locus or an expression product thereof, (3) a CD74 gene locus or an expression product thereof, (4) a 2Q31.3 chromosome locus or an expression product thereof, (5) a 4Q13.1 chromosome locus or an expression product thereof, (6) an ARTS-1 gene or an expression product thereof, (7) an IL-23R gene or an expression product thereof, (8) a TNFR1 gene locus, (9) a TRADD gene locus, (10) chromosome locus 2P15, or (11) chromosome locus 21Q22; and (b) analyzing the sample for a polymorphisms in the AS marker, which is indicative of the presence or risk of development of AS. Any method of screening or detecting the AS-associated polymorphisms within any one or more of the AS markers of the invention is contemplated by the present invention.

However, it will be recognized that while the methods of the present invention are exemplified by the detection of different polymorphisms within the IL-1R1 gene locus, the IL-1R2 gene locus, the CD74 gene locus, the 2Q31.3 and 4Q13.1 chromosome loci, the ARTS-1 gene, the IL-23R gene, the TNFR1 and TRADD gene loci and the 2P15 and 21Q22 chromosome loci, either alone or in combination, any further AS related polymorphisms within those AS marker are also contemplated by the invention. The AS-associated SNPs of the present invention are summarized in Table 1 below.

TABLE 1
AS-ASSOCIATED SNPS

| | | | | | | | Amino |
|---|---|---|---|---|---|---|---|
| | | | | | | | Acid |
| | | | | | | | Change |
| | | Position | Position within | Region of | | | at |
| | Reference | within | GenBank | Gene/ | Gene/ | Base | residue |
| No. | Number | chromosome | Accession No: | locus | locus | Change | No. |
| 1 | rs949963 | Chr 2: | AF531102 | Non- | IL-1R1 | T/C | N/A |
| | | 102136218 | [GI: 22001412] | coding | | | |
| | | | 680; or | | | | |
| | | | SEQ ID NO: 1 | | | | |
| | | | 201 | | | | |
| 2 | rs2310173 | Chr 2: | AC007165 | Non- | IL-1R2 | G/T | N/A |
| | | 102030060 | [GI: 19033999] | coding | | | |
| | | | 45295; or | | | | |
| | | | SEQ ID NO: 2 | | | | |
| | | | 500 | | | | |
| 3 | rs15251 | Chr 5: | NT_029289 | Coding | TCOF1 | T/C | Val/Ala |
| | | 149756425 | 16101 | | (genetically | | 1313¹ |
| | | | (reverse | | linked to | | |
| | | | complement); or | | CD74) | | |
| | | | NG_01134139031; | | | | |
| | | | or | | | | |
| | | | SEQ ID NO: 3 | | | | |
| | | | 401 | | | | |
| 4 | rs1018326 | Chr 2: | NC_000002 | Non- | 2Q31.3 | T/C | N/A |
| | | 181716045 | [GI: 89161199] | coding | (genetically | | |
| | | | 162689; or | | linked to | | |
| | | | SEQ ID NO: 4 | | UBE2E3) | | |
| | | | 301 | | | | |
| 5 | rs10517820 | Chr 4: | AC074087 | Non- | 4Q13.1 | A/G | N/A |
| | | 62963327 | [GI: 15638721] | coding | | | |
| | | | 71341; or | | | | |
| | | | SEQ ID NO: 5 | | | | |
| | | | 101 | | | | |
| 6 | rs27044 | Chr 5 | NC_000005 | Coding | ARTS-1 | C/G | Glu/Gln |
| | | 96144608 | [GI: 224589817] | | | | 730² |
| | | | 25041 reverse | | | | |
| | | | complement | | | | |
| | | | SEQ ID NO: 6 | | | | |
| | | | 301 | | | | |
| 7 | rs17482078 | Chr 5 | NC_000005 | Coding | ARTS-1 | T/C | Gln/Arg |
| | | 96144622 | [GI: 224589817] | | | | 725² |
| | | | 25027 reverse | | | | |
| | | | complement | | | | |
| | | | SEQ ID NO: 7 | | | | |
| | | | 301 | | | | |
| 9 | rs30187 | Chr 5 | NC_000005 | Coding | ARTS-1 | C/T | Arg/Lys |
| | | 96150086 | [GI: 224589817] | | | | 528² |
| | | | 19563 reverse | | | | |
| | | | complement | | | | |
| | | | SEQ ID NO: 8 | | | | |
| | | | 301 | | | | |
| 12 | rs10489629 | Chr 1 | NC_000001 | Non- | IL-23R | C/T | NA |
| | | 67475114 | [GI: 224589800] | coding | | | |
| | | | 56181 | | | | |
| | | | SEQ ID NO: 9 | | | | |
| | | | 101 | | | | |
| 15 | rs1343151 | Chr 1 | NC_000001 | Non- | IL-23R | A/G | NA |
| | | 67497708 | [GI: 224589800] | coding | | | |
| | | | 86961 | | | | |
| | | | SEQ ID NO: 10 | | | | |
| | | | 301 | | | | |
| 16 | rs10889677 | Chr 1 | NC_000001 | Non- | IL-23R | C/A | NA |
| | | 67512680 | [GI: 224589800] | coding | | | |
| | | | 92952 | | | | |
| | | | SEQ ID NO: 11 | | | | |
| | | | 301 | | | | |
| 17 | rs11209032 | Chr 1 | SEQ ID NO: 12 | Non- | IL-23R | G/A | N/A |
| | | 67526096 | 301 | coding | | | |
| 19 | rs4149576 | Chr 12 | NC_000012 | Non- | TNFR1 | T/C | NA |
| | | 6319376 | [GI: 224589803] | coding | | | |
| | | | 2147 | | | | |
| | | | SEQ ID NO: 13 | | | | |
| | | | 301 | | | | |
| 20 | rs9033 | Chr 16 | NC_000016 | Non- | TRADD | A/G | NA |
| | | 65739500 | [GI: 224589807] | coding | | | |
| | | | 38528 | | | | |
| | | | SEQ ID NO: 14 | | | | |
| | | | 300 | | | | |
| 22 | rs2242944 | Chr 21 | SEQ ID NO: 15 | Non- | 21Q22 | A/G | NA |
| | | 39387048 | 501 | coding | | | |

¹Relative to the TCOF1 amino acid sequence set forth in GenPept Accession No. NP_000347 [GI: 57164975]
²Relative to the ARTS-1 amino acid sequence set forth in GenPept Accession No. NP_057526 [GI: 94818901]

The AS-associated SNPs of the present invention may also be used in combination with one or more other AS-associated SNPs, as disclosed for example in International Application No. PCT/AU2008/000762 filed 29 May 2008 (WO 2008/144827), illustrative examples of which are summarized below in Table 2.

TABLE 2
AS-ASSOCIATED SNPS DISCLOSED IN PCT/AU2008/000762

| | | Position | | Region of | | | Amino |
|---|---|---|---|---|---|---|---|
| | Reference | within | Position within | Gene/ | Gene/ | Base | Acid |
| No. | Number | chromosome | SEQ ID NO:* | locus | locus | Change | Change |
| 1 | rs10050860 | Chr 5 | NC_000005 | Coding | ARTS-1 | T/C | Asn/Asp |
| | | 96147966 | [GI: 224589817] | | | | 575 |
| | | | 21683 reverse | | | | |
| | | | complement | | | | |
| | | | SEQ ID NO: 16 | | | | |
| | | | 301 | | | | |
| 2 | rs2287987 | Chr 5 | NC_000005 | Coding | ARTS-1 | C/T | Val/Met |
| | | 96155291 | [GI: 224589817] | | | | 349 |
| | | | 14358 reverse | | | | |
| | | | complement | | | | |
| | | | SEQ ID NO: 17 | | | | |
| | | | 301 | | | | |
| 3 | rs1004819 | Chr 1 | NC_000001 | Non- | IL-23R | G/A | NA |
| | | 67460937 | [GI: 224589800] | coding | | | |
| | | | 38045 | | | | |
| | | | SEQ ID NO: 18 | | | | |
| | | | 489 | | | | |
| 4 | rs11465804 | Chr 1 | NC_000001 | Non- | IL-23R | G/T | NA |
| | | 67478546 | [GI: 224589800] | coding | | | |
| | | | 70358 | | | | |
| | | | SEQ ID NO: 19 | | | | |
| | | | 101 | | | | |
| 5 | rs11209026 | Chr 1 | NC_000001 | Coding | IL-23R | A/G | Gln/Arg |
| | | 67491717 | [GI: 224589800] | | | | 381 |
| | | | 73790 | | | | |
| | | | SEQ ID NO: 20 | | | | |
| | | | 301 | | | | |
| 6 | rs1495965 | Chr 1 | SEQ ID NO: 21 | Non- | IL-23R | T/C | NA |
| | | 67442801 | 259 | coding | | | |
| 7 | rs10865331 | Chr 2 | SEQ ID NO: 22 | Non- | 2P15 | G/A | NA |
| | | 62404976 | 643 | coding | | | |

² Relative to the IL-23R amino acid sequence set forth in GenPept Accession No. NP_653302 [GI: 24430212]

In general, if the polymorphism is located in a gene, it may be located in a non-coding or coding region of the gene. If located in the coding region the polymorphism can result in an amino acid alteration. Such alterations may or may not have an effect on the function or activity of the encoded polypeptide. For example, the polymorphisms listed in Table 2 within the ARTS-1 and IL-23R coding regions are non-synonymous mutations which cause a change in the amino acid sequence. The other seven polymorphisms within the IL-23R gene sequence are in the non-coding region. However, when the polymorphism is located in a non-coding region it can cause alternative splicing, which again, may or may not have an effect on the encoded protein activity or function.

The methods of the present invention comprise detecting the presence or risk of development of AS by identifying related polymorphisms in DNA or mRNA (or on other nucleic acid sequences, such as cDNA, developed there from) or protein contained in tissue, blood or other biological samples taken from a subject. The polymorphism can be detected in any manner conventionally known in the art, e.g., via directly sequencing of the nucleotide sequences contained in the samples. Such diagnosis or prediction can also be made by identifying the nucleotide polymorphism or variant protein in samples taken from kindred or other relatives of a subject. This can be helpful, for example, in determining whether offspring are likely to be genetically predisposed to the condition, even though it has not expressed itself in the parents.

It is to be understood that although the following discussion is specifically directed to human subjects, the teachings are also applicable to any animal that expresses a transcript thereof in accordance with the present invention, such that clinical manifestations such as those seen in subjects with AS are found.

It will be appreciated that the methods described herein are applicable to any subject suspected of developing, or having AS, whether the condition is manifest at a young age or at a more advanced age in a patient's life. The subject can be an adult, child, fetus or embryo.

The diagnostic and screening methods of the invention are especially useful for a subject suspected of being at risk of developing AS based on family history, or a subject in which it is desired to diagnose or eliminate the presence of AS as a causative agent underlying a subject's symptoms.

### 3. Screening for Specific Polymorphisms within the AS Markers of the Invention

3.1 Amplification Techniques

In some embodiments, screening or diagnosis of AS, or a predisposition to developing AS in a subject is now possible by detecting a polymorphism linked to that condition. For example, numerous methods are known in the art for determining the nucleotide occurrence at a particular position corresponding to a single nucleotide polymorphism in a sample. Suitably, methods of detecting point mutations may be accomplished by molecular cloning of the specified allele and subsequent sequencing of that allele using techniques well known in the art. A method according to the present invention can identify a nucleotide occurrence for either strand of DNA. Additionally, the gene sequences may be amplified directly from a DNA or mRNA (or on other nucleic acid sequences, such as cDNA) preparation from the sample using amplification techniques, and the sequence composition can then be determined from the amplified product.

The nucleic acid sample may be obtained from any part of the subject's body, including, but not limited to hair, skin, nails, tissues or bodily fluids such as saliva and blood. The subject for the methods of the present invention can be a subject of any race or national origin.

Nucleic acid isolation protocols are well known to those of skill in the art. For example, an isolated polynucleotide corresponding to a gene or allele or chromosome region (e.g., as listed in Tables 1 and 2) may be prepared according to the following procedure:

creating primers which flank an allele or transcript thereof, or a portion of the allele or transcript;

obtaining a nucleic acid extract from an individual affected with, or at risk of developing AS;

and using the primers to amplify, via nucleic acid amplification techniques, at least one amplification product from the nucleic acid extract, wherein the amplification product corresponds to the allele or transcript linked to the development of the condition.

Suitable nucleic acid amplification techniques are well known to a person of ordinary skill in the art, and include polymerase chain reaction (PCR) as for example described in Ausubel et al., Current Protocols in Molecular Biology (John Wiley & Sons, Inc. 1994-1998) strand displacement amplification (SDA) as for example described in U.S. Pat. No. 5,422,252; rolling circle replication (RCR) as for example described in Liu et al., (1996, J. Am. Chem. Soc. 118: 1587-1594 and International application WO 92/01813) and Lizardi et al., (International Application WO 97/19193); nucleic acid sequence-based amplification (NASBA) as for example described by Sooknanan et al., (1994, Biotechniques 17: 1077-1080); ligase chain reaction (LCR); simple sequence repeat analysis (SSR); branched DNA amplification assay (b-DNA); transcription amplification and self-sustained sequence replication; and Q-13 replicase amplification as for example described by Tyagi et al., (1996, Proc. Natl. Acad. Sci. USA 93: 5395-5400).

Such methods can utilize one or more oligonucleotide probes or primers, including, for example, an amplification primer pair, that selectively hybridize to a target polynucleotide, which contains one or more SNPs. Oligonucleotide probes useful in practicing a method of the invention can include, for example, an oligonucleotide that is complementary to and spans a portion of the target polynucleotide, including the position of the SNP, wherein the presence of a specific nucleotide at the polymorphic site (i.e., the SNP) is detected by the presence or absence of selective hybridization of the probe. Such a method can further include contacting the target polynucleotide and hybridized oligonucleotide with an endonuclease, and detecting the presence or absence of a cleavage product of the probe, depending on whether the nucleotide occurrence at the polymorphic site is complementary to the corresponding nucleotide of the probe.

Primers may be manufactured using any convenient method of synthesis. Examples of such methods may be found in “Protocols for Oligonucleotides and Analogues; Synthesis and Properties”, Methods in Molecular Biology Series; Volume 20; Ed. Sudhir Agrawal, Humana ISBN: 0-89603-247-7; 1993. The primers may also be labeled to facilitate detection.

3.2 Nucleic Acid Polymorphism Screening Techniques

Various tools for the detection of polymorphisms within a target DNA are known in the art, including, but not limited to screening techniques, DNA sequencing, scanning techniques, hybridization based techniques, extension based analysis, incorporation based techniques, restriction enzyme based analysis and ligation based techniques.

3.3 Nucleic Acid Sequencing Techniques

In some embodiments, the polymorphism is identified through nucleic acid sequencing techniques. Specifically, amplification products which span a SNP locus can be sequenced using traditional sequence methodologies (e.g., the “dideoxy-mediated chain termination method”, also known as the “Sanger Method” (Sanger, F., et al., 1975, J. Molecular, Biol. 94: 441; Prober et al., 1987, Science, 238: 336-340) and the “chemical degradation method”, also known as the “Maxam-Gilbert method” (Maxam, A. M., et al., 1977, Proc. Natl. Acad. Sci. (U.S.A.) 74: 560), both references herein incorporated by reference to determine the nucleotide occurrence at the SNP loci.

Boyce-Jacino, et al., U.S. Pat. No. 6,294,336 provides a solid phase sequencing method for determining the sequence of nucleic acid molecules (either DNA or RNA) by utilizing a primer that selectively binds a polynucleotide target at a site wherein the SNP is the most 3′ nucleotide selectively bound to the target. Other sequencing technologies such as Denaturing High Pressure Liquid Chromatography or mass spectroscopy may also be employed.

In other illustrative examples, the sequencing method comprises a technique known as Pyrosequencing™. The approach is based on the generation of pyrophosphate whenever a deoxynucleotide is incorporated during polymerization of DNA. The generation of pyrophosphate is coupled to a luciferase catalysed reaction resulting in light emission if the particular deoxynucleotide added is incorporated, yielding a quantitative and distinctive pyrogram. Sample processing includes PCR amplification with a biotinylated primer, isolation of the biotinylated single strand amplicon on streptavidin coated beads (or other solid phase) and annealing of a sequencing primer. Samples are then analysed by a Pyrosequencer™ which adds a number of enzymes and substrates required for the indicator reaction, including sulfurylase and luciferase, as well as apyrase for degradation of unincorporated nucleotides. The sample is then interrogated by addition of the four deoxynucleotides. Light emission can be detected by a charge coupled device camera (CCD) and is proportional to the number of nucleotides incorporated. Results are automatically assigned by pattern recognition.

Alternatively, methods of the invention can identify nucleotide occurrences at polymorphic sites within a nucleic acid sequence using a “micro-sequencing” method. Micro-sequencing methods determine the identity of only a single nucleotide at a “predetermined” site. Such methods have particular utility in determining the presence and identity of polymorphisms in a target polynucleotide. Such micro-sequencing methods, as well as other methods for determining the nucleotide occurrence at a polymorphic site are discussed in Boyce-Jacino et al., U.S. Pat. No. 6,294,336, incorporated herein by reference.

Micro-sequencing methods include the Genetic Bit Analysis™ method disclosed by Goelet, P. et al. WO 92/15712. Additional, primer-guided, nucleotide incorporation procedures for assaying polymorphic sites in DNA have also been described (Komher, J. S. et al, 1989, Nucl. Acids. Res. 17: 7779-7784; Sokolov, B. P., 1990, Nucl. Acids Res. 18: 3671; Syvanen, A. C, et al., 1990, Genomics, 8: 684-692; Kuppuswamy, M. N. et al., 1991, Proc. Natl. Acad. Sci. (U.S.A.) 88: 1143-1147; Prezant, T. R. et al, 1992, Hum. Mutat. 1: 159-164; Ugozzoli, L. et al., 1992, GATA, 9: 107-112; Nyren, P. et al., 1993, Anal. Biochem. 208: 171-175; and Wallace, WO89/10414). These methods differ from Genetic Bit™ analysis in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A. C., et al., 1993, Amer. J. Hum. Genet. 52: 46-59).

Further micro-sequencing methods have been provided by Mundy, C. R. (U.S. Pat. No. 4,656,127) and Cohen, D. et al (French Patent 2,650,840; PCT Application. No. WO91/02087) which discusses a solution-based method for determining the identity of a nucleotide of a polymorphic site. As in the Mundy method of U.S. Pat. No. 4,656,127, a primer is employed that is complementary to allelic sequences immediately 3′ to a polymorphic site.

In other illustrative examples, Macevicz (U.S. Pat. No. 5,002,867), for example, describes a method for determining nucleic acid sequences via hybridization with multiple mixtures of oligonucleotide probes. In accordance with such methods, the sequence of a target polynucleotide is determined by permitting the target to sequentially hybridize with sets of probes having an invariant nucleotide at one position, and a variant nucleotides at other positions. The Macevicz method determines the nucleotide sequence of the target by hybridizing the target with a set of probes, and then determining the number of sites that at least one member of the set is capable of hybridizing to the target (i.e., the number of “matches”). This procedure is repeated until each member of a set of probes has been tested.

Alternatively, the template-directed dye-terminator incorporation assay with fluorescence polarization detection (FP-TDI) assay (Chen et al., 1999) is a version of the primer extension assay that is also called mini-sequencing or the single base extension assay (Syvanen, 1994). The primer extension assay is capable of detecting SNPs. The DNA sequencing protocol ascertains the nature of the one base immediately 3′ to the SNP-specific sequencing primer that is annealed to the target DNA immediately upstream from the polymorphic site. In the presence of DNA polymerase and the appropriate dideoxyribonucleoside triphosphate (ddNTP), the primer is extended specifically by one base as dictated by the target DNA sequence at the polymorphic site. By determining which ddNTP is incorporated, the allele(s) present in the target DNA can be inferred.

3.4 Polymorphism Scanning Techniques

Scanning techniques contemplated by the present invention for detecting polymorphisms within a nucleotide sequence can include, but are not restricted to, chemical mismatch cleavage (CMC) (Saleeba, J. A et al., 1992, Huma. Mutat, 1: 63-69), mismatch repair enzymes cleavage (MREC) (Lu, A. L and Hsu, I. C., 1992, Genomics, 14(2): 249-255), chemical cleavage techniques, denaturing gradient gel electrophoresis (DGGE) Wartell et al., (1990, Nucl. Acids Res. 18: 2699-2705 and; Sheffield et al., 1989, Proc. Natl. Acad. Sci. USA 86: 232-236), temperature gradient gel electrophoresis (TGGE) (Salimullah, et al., 2005, Cellular and Mol. Biol. Letts, 10: 237-245), constant denaturant gel electrophoresis (CDGE), single strand conformation polymorphism (SSCP) analysis (Kumar, D et al., 2006, Genet. Mol. Biol, 29(2): 287-289), heteroduplex analysis (HA) (Nagamine, C. M et al., 1989, Am. J. Hum. Genet, 45: 337-339), microsatellite marker analysis and single strand polymorphism assays (SSPA).

In some embodiments, the SNPs of the present invention are detected through CMC, wherein a radio-labeled DNA wild type sequence (probe) is hybridized to an amplified sequence containing the putative alteration to form a heteroduplex. A chemical modification, followed by piperidine cleavage, is used to remove the mismatch bubble in the heteroduplex. Gel electrophoresis of the denatured heteroduplex and autoradiography allow to visualize the cleavage product. Osmium tetroxide is used for the modification of mispaired thymidines and hydroxylamine for mismatched cytosines. Additionally, labeling the antisense strand of the probe DNA allows the detection of adenosine and guanosine mismatches. The chemical cleavage of mismatch can be used to detect almost 100% of mutations in long DNA fragments. Moreover, this method provides the precise characterization and the exact location of the mutation within the tested fragment. Recently, the method has been amended to make CMC more suitable for automation by using fluorescent primers also enabling multiplexing and thereby reducing the number of manipulations. Alternatively, fluorescently labeled dUTPs incorporated via PCR allow the internal labeling of both target and probe DNA strands and therefore labeling of each possible hybrid, doubling the chances of mutation detection and virtually guaranteeing 100% detection.

In other embodiments, the mismatch repair enzymes cleavage (MREC) assay is used to identify single base substitutions within an AS marker of the present invention. MREC relies on nicking enzyme systems specific for mismatch-containing DNA. The sequence of interest is amplified by PCR and homo- and heteroduplex species may be generated at the end of the PCR, by denaturing and allowing to re-anneal the amplified products. These hybrids are treated with mismatch repair enzymes and then analysed by denaturing gel electrophoresis. The MREC assay makes use of three mismatch repair enzymes. The MutY endonuclease removes adenines from the mismatches and is useful to detect both A/T and CIG transversions and G/C and T/A transitions. Mammalian thymine glycosylase removes thymines from T/G, T/C, and T/T mismatches and is useful to detect G/C and A/T transitions as well as A/T and G/C and T/A and A/T transversions. The all-type endonuclease or topoisomerase I from human or calf thymus can recognize all eight mismatches and can be used to scan any nucleotide substitution. MREC can use specific labels which can be incorporated into both DNA strands, thus allowing all four possible nucleotide substitutions in a give site to be identified.

In some embodiments, chemical cleavage analysis as described in U.S. Pat. No. 5,217,863 (by R. G. H. Cotton) is used for identifying SNPs within nucleotide sequences. Like heteroduplex analysis, chemical cleavage detects different properties that result when mismatched allelic sequences hybridize with each other. Instead of detecting this difference as an altered migration rate on a gel, the difference is detected in altered susceptibility of the hybrid to chemical cleavage using, for example, hydroxylamine, or osmium tetroxide, followed by piperidine.

Among the cleavage methods contemplated by the present invention, RNAse A relies on the principle of heteroduplex mismatch analysis. In the RNAse A cleavage method, RNA-DNA heteroduplex between radiolabeled wild-type riboprobe and a mutant DNA, obtained by PCR amplification, is enzymatically cleaved by RNAse A, by exploiting the ability of RNAse A to cleave single-stranded RNA at the points of mismatches in RNA:DNA hybrids. This is followed by electrophoresis and autoradiography. The presence and location of a mutation are indicated by a cleavage product of a given size (Meyers, R. M et al., 1985, Science, 230: 1242-1246 and; Gibbs, R. A and Caskey, T, 1987, Science, 236: 303-305).

The riboprobe need not be the full-length of an AS marker sequences of the present invention (e.g., as listed in Tables 1 and 2). However, a number of probes can be used to screen the whole mRNA sequence for mismatches. In a similar fashion, DNA probes can be used to detect mismatches, through enzymatic or chemical cleavage. See, e.g., Cotton, et al., 1988, Proc. Natl. Acad. Sci. USA 85: 4397; Shenk et al., 1975, Proc. Natl. Acad. Sci. USA 72: 989; and Novack et al., 1986, Proc. Natl. Acad. Sci. USA 83: 586.

In some embodiments, the Invader® assay (Third Wave™ Technology) is employed to scan for polymorphisms within the AS marker sequences of the present invention. For example, the Invader® assay is based on the specificity of recognition, and cleavage, by a Flap endonuclease, of the three dimensional structure formed when two overlapping oligonucleotides hybridize perfectly to a target DNA (Lyamichev, V et al., 1999, Nat Biotechnol, 17: 292-296).

Alternatively, denaturing gradient gel electrophoresis (DGGE) is a useful technique to separate and identify sequence variants. DGGE is typically performed in constant-concentration polyacrylamide gel slabs, cast in the presence of linearly increasing amounts of a denaturing agent (usually formamide and urea, cathode to anode). A variant of DGGE employs temperature gradients along the migration path and is known as TGGE. Separation by DGGE or TGGE is based on the fact that the electrophoretic mobility in a gel of a partially melted DNA molecule is greatly reduced as compared to an unmelted molecule.

In some embodiments, constant denaturant gel electrophoresis (CDGE) is useful for detecting SNPs within a nucleotide sequence, as described in detail in Smith-Sorenson et al., 1993, Human Mutation 2: 274-285 (see also, Anderson & Borreson, 1995, Diagnostic Molecular Pathology 4: 203-211). A given DNA duplex melts in a predetermined, characteristic fashion in a gel of a constant denaturant. Mutations alter this movement. An abnormally migrating fragment is isolated and sequenced to determine the specific mutation.

In other embodiments, single-strand conformation polymorphism (SSCP) analysis provides a method for detecting SNPs within the AS marker sequences of the present invention. SSCP is a method based on a change in mobility of separated single-strand DNA molecules in non-denaturing polyacrylamide gel electrophoresis. Electrophoretic mobility depends on both size and shape of a molecule, and single-stranded DNA molecules fold back on themselves and generate secondary structures which are determined by intra-molecular interactions in a sequence dependent manner. A single nucleotide substitution can alter the secondary structure and, consequently, the electrophoretic mobility of the single strands, resulting in band shifts on autoradiographs. The ability of a given nucleotide variation to alter the conformation of the single strands is not predictable on the basis of an adequate theoretical model and base changes occurring in a loop or in a long stable stem of the secondary structure might not be detected by SSCP. Standard SSCP reaches maximal reliability in detecting sequence alterations in fragments of 150-200 bp. More advanced protocols, allowing the detection of mutations at sensitivity equal to that of the radioactively-based SSCP analysis, have been developed. These methods use fluorescence-labeled primers in the PCR and analyze the products with a fluorescence-based automated sequencing machine. Multi-colour fluorescent SSCP also allows to include an internal standard in every lane, which can be used to compare data from each lane with respect to each other. Other variants to increase the detection rate includes a dideoxy sequencing approach based on dideoxy fingerprinting (ddF) and restriction endonuclease fingerprinting (REF).

The method of ddF is a combination of SSCP and Sanger dideoxy sequencing which involves non-denaturing gel electrophoresis of a Sanger sequencing reaction with one dideoxynucleotide. In this way, for example, a 250-bp fragment can be screened to identify a SNP. REF is a more complex modification of SSCP allowing the screening of more than 1 kb fragments. For REF, a target sequence is amplified with PCR, digested independently with five to six different restriction endonucleases and analyzed by SSCP on a non-denaturing gel. In the case of six restriction enzymes being used, a sequence variation will be present in six different restriction fragments, thus generating 12 different single-stranded segments. A mobility shift in any one of these fragments is sufficient to pinpoint the presence of a sequence variation within a portion of at least one of the AS marker sequences of the invention. The restriction pattern obtained enables localization of an alteration in the region examined.

In some embodiments, heteroduplex analysis (HA) detects single base substitutions in PCR products or nucleotide sequences. HA can be rapidly performed without radioisotopes or specialized equipment. The HA method takes advantage of the formation of heteroduplexes between wild-type and mutated sequences by heating and renaturing of PCR products. Due to a more open double-stranded configuration surrounding the mismatched bases, heteroduplexes migrate slower than their corresponding homoduplexes, and are then detected as bands of reduced mobility compared to normal and mutant homoduplexes on polyacrylamide gels. The ability of a particular single base substitution to be detected by the HA method cannot be predicted merely by knowing the mismatched bases since the adjacent nucleotides have a substantial effect on the configuration of the mismatched region and length-based separation will clearly miss nucleotide substitutions. Optimization of the temperature, gel cross-linking and concentration of acrylamide used as well as glycerol and sucrose enhance the resolution of mutated samples. The HA method can be rapidly performed without radioisotopes or specialized equipment and screens large numbers of samples from subjects for known mutations and polymorphisms in sequenced genes. When HA is used in combination with SSCP, up to 100% of all alterations in a DNA fragment can be easily detected.

In some embodiments, the use of proteins which recognize nucleotide mismatches, such as the E. coli mutS protein can be used to detect an AS-associated polymorphism within at least one of the AS marker sequences of the present invention (Modrich, 1991, Ann. Rev. Genet. 25: 229-253). In the mutS assay, the protein binds only to sequences that contain a nucleotide mismatch in a heteroduplex between mutant and wild-type sequences.

In further embodiments, polymorphism detection can be performed using microsatellite marker analysis. Microsatellite markers with an average genome spacing, for example of about 10 centimorgans (cM) can be employed using standard DNA isolation methods known in the art.

SSPA analysis and the closely related heteroduplex analysis methods described above may be used for screening for single-base polymorphisms (Orita, M. et al., 1989, Proc Natl Acad Sci USA, 86: 2766). In these methods, the mobility of PCR-amplified test DNA from subjects with AS or at risk of developing AS is compared with the mobility of DNA amplified from normal sources by direct electrophoresis of samples in adjacent lanes of native polyacrylamide or other types of matrix gels. Single-base changes often alter the secondary structure of the molecule sufficiently to cause slight mobility differences between the normal and mutant PCR products after prolonged electrophoresis. The presence of polymorphisms, including mutations, in nucleic acids by using mass spectrometry may be used as discussed in U.S. Pat. No. 5,869,242.

3.5 Polymorphism Hybridization Based Techniques

Hybridization techniques for detecting polymorphisms within a nucleotide sequence can include, but are not restricted to the TaqMan® assay (Applied Biosystems), dot blots, reverse dot blot, Multiplex-allele-specific diagnostic assays (MASDA), Dynamic allele-specific hybridization (DASH) Jobs et al., (2003, Genome Res 13: 916-924), molecular beacons and Southern blots.

The TaqMan® assay for identifying SNPs within a nucleotide sequence is based on the nuclease activity of Taq polymerase that displaces and cleaves the oligonucleotide probes hybridized to the target DNA, generating a fluorescent signal. Two TaqMan® probes that differ at the polymorphic site are required; one probe is complementary to the wild-type allele and the other to the variant allele. The probes have different fluorescent dyes attached to the 50 end and a quencher attached to the 30 end. When the probes are intact, the quencher interacts with the fluorophore by fluorescence resonance energy transfer (FRET), quenching their fluorescence. During the PCR annealing step, the TaqMan® probes hybridize to the target DNA. In the extension step, the fluorescent dye is cleaved by the nuclease activity of the Taq polymerase, leading to an increase in fluorescence of the reporter dye. Mismatch probes are displaced without fragmentation. The genotype of a sample is determined by measuring the signal intensity of the two different dyes.

In some embodiments, a biological sample from a subject can be probed in a standard dot blot format. Each region within the test sample that contains a nucleotide sequence corresponding to the AS marker sequences or a portion of is individually applied to a solid surface, for example, as an individual dot on a membrane. Each individual region can be produced, for example, as a separate PCR amplification product using methods well-known in the art (see, for example, the experimental embodiment set forth in Mullis, K. B., 1987, U.S. Pat. No. 4,683,202).

In a related embodiment, a reverse dot blot format is employed, wherein oligonucleotide or polynucleotide probes having known sequence are immobilized on the solid surface, and are subsequently hybridized with the labeled test polynucleotide sample.

Another useful SNP identification method includes DASH (dynamic allele-specific hybridization), which encompasses dynamic tracking of probe (oligonucleotide) to target (PCR product) hybridization as the reaction temperature is steadily increased to identify polymorphisms (Prince, J. A et al., 2001, Genome Res, 11(1): 152-162).

In some embodiments, multiplex-allele-specific diagnostic assays (MASDA) can be used for the analysis of a large number of samples (>500). MASDA utilizes oligonucleotide hybridization to interrogate DNA sequences. Multiplex DNA samples are immobilized on a solid support and a single hybridization is performed with a pool of allele-specific oligonucleotide (ASO) probes. Any probes complementary to specific mutations present in a given sample are in effect affinity purified from the pool by the target DNA. Sequence-specific band patterns (fingerprints), generated by chemical or enzymatic sequencing of the bound ASO(s), easily identify the specific mutation(s).

There are several alternative hybridization-based techniques, including, among others, molecular beacons, and Scorpion® probes (Tyagi, S. and Kramer, F. R., 1996, Nat. Biotechnol, 14: 303-308; Thelwell et al., 2000, Nucleic Acid Res. 28(19): 3752-3761). Molecular beacons are comprised of oligonucleotides that have a fluorescent reporter and quencher dyes at their 5′ and 3′ ends. The central portion of the oligonucleotide hybridizes across the target sequence, but the 5′ and 3′ flanking regions are complementary to each other. When not hybridised to their target sequence, the 5′ and 3′ flanking regions hybridise to form a stem-loop structure, and there is little fluorescence because of the proximity of the reporter and quencher dyes. However, upon hybridization to their target sequence, the dyes are separated and there is a large increase in fluorescence. Mismatched probe-target hybrids dissociate at substantially lower temperature than exactly complementary hybrids. There are a number of variations of the “beacon” approach. Scorpion® probes are similar but incorporate a PCR primer sequence as part of the probe. A more recent “duplex” format has also been developed.

In some embodiments, a further method of identifying an SNP comprises the SNP-IT™ method (Orchid BioSciences, Inc., Princeton, N.J.). In general, SNP-IT™ is a 3-step primer extension reaction. In the first step a target polynucleotide is isolated from a sample by hybridization to a capture primer, which provides a first level of specificity. In a second step the capture primer is extended from a terminating nucleotide trisphosphate at the target SNP site, which provides a second level of specificity. In a third step, the extended nucleotide trisphosphate can be detected using a variety of known formats, including: direct fluorescence, indirect fluorescence, an indirect colorimetric assay, mass spectrometry, fluorescence polarization, etc. Reactions can be processed in 384 well format in an automated format using a SNPstream™ instrument (Orchid BioSciences, Inc., Princeton, N.J.).

In these embodiments, the amplification products can be detected by Southern blot analysis with or without using radioactive probes. In one such method, for example, a small sample of DNA containing a very low level of the nucleic acid sequence of the polymorphic locus is amplified, and analyzed via a Southern blotting technique or similarly, using dot blot analysis. The use of non-radioactive probes or labels is facilitated by the high level of the amplified signal. Alternatively, probes used to detect the amplified products can be directly or indirectly detectably labeled, for example, with a radioisotope, a fluorescent compound, a bioluminescent compound, a chemiluminescent compound, a metal chelator or an enzyme.

Hybridization conditions, such as salt concentration and temperature can be adjusted for the nucleotide sequence from a subject suspected of having AS or being at risk of developing AS, to be screened. Southern blotting and hybridizations protocols are described in Current Protocols in Molecular Biology (Greene Publishing Associates and Wiley-Interscience), pages 2.9.1-2.9.10. Probes can be labeled for hybridization with random oligomers and the Klenow fragment of DNA polymerase. Very high specific activity probes can be obtained using commercially available kits such as the Ready-To-Go DNA Labeling Beads (Pharmacia Biotech), following the manufacturer's protocol. Possible competition of probes having high repeat sequence content, and stringency of hybridization and wash down will be determined individually for each probe used. Alternatively, fragments of a candidate sequence may be generated by PCR, the specificity may be verified using a rodent-human somatic cell hybrid panel, and sub-cloning the fragment. This allows for a large prep for sequencing and use as a probe. Once a given gene fragment has been characterized, small probe preparations can be achieved by gel or column purifying the PCR product.

Suitable materials that can be used in the dot blot, reverse dot blot, multiplex, and MASDA formats are well-known in the art and include, but are not limited to nylon and nitrocellulose membranes.

3.6 Nucleotide Arrays and Gene Chips for Polymorphism Analysis

The invention further contemplates methods of identifying SNPs through the use of an array of oligonucleotides, wherein discrete positions on the array are complementary to one or more of the provided polymorphic sequences, e.g. oligonucleotides of at least 12 nt, at least about 15 nt, at least about 18 nt, at least about 20 nt, or at least about 25 nt, or longer, and including the sequence flanking the polymorphic position. Such an array may comprise a series of oligonucleotides, each of which can specifically hybridize to a different polymorphism. For examples of arrays, see Hacia et al. (1996, Nat. Genet. 14: 441-447 and De Risi et al., (1996, Nat. Genet. 14: 457-460).

A nucleotide array can include all or a subset of the polymorphisms of the invention. One or more polymorphic forms may be present in the array. In some embodiments, an array includes at least 2 different polymorphic sequences, i.e., polymorphisms located at unique positions within the AS marker sequences of the present invention, and may include as many of the provided polymorphisms as required. Arrays of interest may further comprise sequences, including polymorphisms, of other genetic sequences, particularly other sequences of interest for pharmacogenetic screening, including, but not limited to, other genes associated with AS. The oligonucleotide sequence on the array is generally at least about 12 nt in length, at least about 15 nt, at least about 18 nt, at least about 20 nt, or at least about 25 nt, or may be the length of the provided polymorphic sequences, or may extend into the flanking regions to generate fragments of 100 to 200 nt in length. For examples of arrays, see Ramsay (1998, Nature Biotech. 16: 40-44; Hacia et al., (1996, Nature Genetics 14: 441-447; Lockhart et al., (1996, Nature Biotechnol. 14:1675-1680; and De Risi et al., (1996, Nature Genetics 14: 457-460).

A number of methods are available for creating micro-arrays of biological samples, such as arrays of DNA samples to be used in DNA hybridization assays. Examples of such arrays are discussed in detail in PCT Application number. WO95/35505 (1995); U.S. Pat. No. 5,445,934, (1995); and Drmanac et al., (1993, Science 260:1649-1652). Yershov et al., (1996, Genetics 93: 4913-4918) describe an alternative construction of an oligonucleotide array. The construction and use of oligonucleotide arrays is reviewed by Ramsay (1998) supra.

Methods of using high density oligonucleotide arrays for identifying polymorphisms within nucleotide sequences are known in the art. For example, Milosavljevic et al., (1996, Genomics 37: 77-86) describe DNA sequence recognition by hybridization to short oligomers. See also, Drmanac et al., (1998, Nature Biotech. 16: 54-58; and Drmanac and Drmanac, 1999, Methods Enzymol. 303: 165-178). The use of arrays for identification of unknown mutations is proposed by Ginot, (1997, Human Mutation 10:1-10).

Detection of known mutations is described in Hacia et al. (1996, Nat. Genet. 14: 441-447; Cronin et al., (1996) Human Mut. 7: 244-255; and others. The use of arrays in genetic mapping is discussed in Chee et al., (1996, Science 274: 610-613; Sapolsky and Lishutz, 1996, Genomics 33: 445-456; and Shoemaker et al., 1996, Nat. Genet. 14: 450-456) perform quantitative phenotypic analysis of yeast deletion mutants using a parallel bar-coding strategy.

Quantitative monitoring of gene expression patterns with a complementary DNA microarray is described in Schena et al., (1995, Science 270: 467; DeRisi et al., 1997, Science 270: 680-686) explore gene expression on a genomic scale. Wodicka et al., (1997, Nat. Biotech. 15: 1-15) perform genome wide expression monitoring in S. cerevisiae.

A DNA sample for analysis is prepared in accordance with conventional methods, e.g., lysing cells, removing cellular debris, separating the DNA from proteins, lipids or other components present in the mixture and then using the isolated DNA for cleavage. See Molecular Cloning, A Laboratory Manual, 2nd ed. (eds. Sambrook et al.) CSH Laboratory Press, Cold Spring Harbor, N.Y. 1989. Generally, at least about 0.5 μg of DNA will be employed, usually at least about 5 μg of DNA, while less than 50 μg of DNA will usually be sufficient.

The nucleic acid samples are cleaved to generate probes. It will be understood by one of skill in the art that any method of random cleavage will generate a distribution of fragments, varying in the average size and standard deviation. Usually the average size will be at least about 12 nucleotides (nts) in length, more usually at least about 20 nts in length, and preferably at least about 35 nts in length. Where the variation in size is great, conventional methods may be used to remove the large and/or small regions of the fragment population.

It is desirable, but not essential to introduce breaks randomly, with a method which does not act preferentially on specific sequences. Preferred methods produce a reproducible pattern of breaks. Methods for introducing random breaks or nicks in nucleic acids include but are not restricted to reaction with Fenton reagent to produce hydroxyl radicals and other chemical cleavage systems, integration mediated by retroviral integrase, partial digestion with an ultra-frequent cutting restriction enzyme, partial digestion of single stranded DNA with SI nuclease, partial digestion with DNAseI in the absence or presence of Mn⁺⁺, etc.

The fragmented nucleic acid samples are denatured and labeled. Labeling can be performed according to methods well known in the art, using any method that provides for a detectable signal either directly or indirectly from the nucleic acid fragment. In a preferred embodiment, the fragments are end-labeled, in order to minimize the steric effects of the label. For example, terminal transferase may be used to conjugate a labeled nucleotide to the nucleic acid fragments. Suitable labels include biotin and other binding moieties; fluorochromes, e.g., fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N,N-tetramethyl-6-carboxyrhodamine (TAMRA), and the like. Where the label is a binding moiety, the detectable label is conjugated to a second stage reagent, e.g., avidin, streptavidin, etc., that specifically binds to the binding moiety, for example a fluorescent probe attached to streptavidin. Incorporation of a fluorescent label using enzymes such as reverse transcriptase or DNA polymerase, prior to fragmentation of the sample, is also possible.

Each of the labeled genome samples is separately hybridized to an array of oligonucleotide probes. Hybridization of the labeled sequences is accomplished according to methods well known in the art. Hybridization can be carried out under conditions varying in stringency, preferably under conditions of high stringency, e.g., 6×SSPE, at 65° C., to allow for hybridization of complementary sequences having extensive homology, usually having no more than one or two mismatches in a probe of 25 nts in length, i.e., at least 95% to 100% sequence identity.

High density microarrays of oligonucleotides are known in the art and are commercially available. The sequence of oligonucleotides on the array will correspond to a known target sequences. The length of oligonucleotide present on the array is an important factor in how sensitive hybridization will be to the presence of a mismatch. Usually oligonucleotides will be at least about 12 nt in length, more usually at least about 15 nt in length, preferably at least about 20 nt in length and more preferably at least about 25 nt in length, and will be not longer than about 35 nt in length, usually not more than about 30 nt in length.

Methods of producing large arrays of oligonucleotides are described in U.S. Pat. No. 5,134,854 (Pirrung et al.), and U.S. Pat. No. 5,445,934 (Fodor et al.) using light-directed synthesis techniques. Using a computer controlled system, a heterogeneous array of monomers is converted, through simultaneous coupling at a number of reaction sites, into a heterogeneous array of polymers. Alternatively, microarrays are generated by deposition of pre-synthesized oligonucleotides onto a solid substrate, for example as described in International Publication WO 95/35505.

Microarrays can be scanned to detect hybridization of the labeled genome samples. Methods and devices for detecting fluorescently marked targets on devices are known in the art. Generally such detection devices include a microscope and light source for directing light at a substrate. A photon counter detects fluorescence from the substrate, while an x-y translation stage varies the location of the substrate. A confocal detection device that may be used in the subject methods is described in U.S. Pat. No. 5,631,734. A scanning laser microscope is described in Shalon et al., (1996, Genome Res. 6: 639). A scan, using the appropriate excitation line, is performed for each fluorophore used. The digital images generated from the scan are then combined for subsequent analysis. For any particular array element, the ratio of the fluorescent signal from one Nucleic acid sample is compared to the fluorescent signal from the other Nucleic acid sample, and the relative signal intensity determined.

Methods for analyzing the data collected by fluorescence detection are known in the art. Data analysis includes the steps of determining fluorescent intensity as a function of substrate position from the data collected, removing outliers, i.e., data deviating from a predetermined statistical distribution, and calculating the relative binding affinity of the targets from the remaining data. The resulting data may be displayed as an image with the intensity in each region varying according to the binding affinity between targets and probes.

Nucleic acid analysis via microchip technology is also applicable to the present invention. In this technique, thousands of distinct oligonucleotide probes can be applied in an array on a silicon chip. A nucleic acid to be analyzed is fluorescently labeled and hybridized to the probes on the chip. It is also possible to study nucleic acid-protein interactions using these nucleic acid microchips. Using this technique one can determine the presence of mutations, sequence the nucleic acid being analyzed, or measure expression levels of a gene of interest. The method is one of parallel processing of many, even thousands, of probes at once and can tremendously increase the rate of analysis.

Alteration of mRNA transcription can be detected by any techniques known to persons of ordinary skill in the art. These include Northern blot analysis, PCR amplification and RNase protection. Diminished mRNA transcription indicates an alteration of the sequence.

The array/chip technology has already been applied with success in numerous cases. For example, the screening of mutations has been undertaken in the BRCA 1 gene, in S. cerevisiae mutant strains, and in the protease gene of HIV-1 virus (Hacia et al., 1996; Shoemaker et al., 1996; Kozal et al., 1996). Chips of various formats for use in detecting SNPs can be produced on a customized basis.

An array-based tiling strategy useful for detecting SNPs is described in EP 785280. Briefly, arrays may generally be “tiled” for a large number of specific polymorphisms. “Tiling” refers to the synthesis of a defined set of oligonucleotide probes that are made up of a sequence complementary to the target sequence of interest, as well as preselected variations of that sequence, e.g., substitution of one or more given positions with one or more members of the basis set of monomers, i.e., nucleotides. Tiling strategies are further described in PCT application No. WO 95/11995. In some embodiments, arrays are tiled for a number of specific SNPs. In particular, the array is tiled to include a number of detection blocks, each detection block being specific for a specific SNP or a set of SNPs. For example, a detection block may be tiled to include a number of probes that span the sequence segment that includes a specific SNP. To ensure probes that are complementary to each allele, the probes are synthesized in pairs differing at the SNP position. In addition to the probes differing at the SNP position, monosubstituted probes are also generally tiled within the detection block. Such methods can readily be applied to the SNP information disclosed herein.

These monosubstituted probes have bases at and up to a certain number of bases in either direction from the polymorphism, substituted with the remaining nucleotides (selected from A, T, G, C and U). Typically, the probes in a tiled detection block will include substitutions of the sequence positions up to and including those that are 5 bases away from the SNP. The monosubstituted probes provide internal controls for the tiled array, to distinguish actual hybridization from artifactual cross-hybridization. Upon completion of hybridization with the target sequence and washing of the array, the array is scanned to determine the position on the array to which the target sequence hybridizes. The hybridization data from the scanned array is then analyzed to identify which allele or alleles of the SNP are present in the sample. Hybridization and scanning may be carried out as described in PCT application No. WO 92/10092 and WO 95/11995 and U.S. Pat. No. 5,424,186.

Thus, in some embodiments, the chips may comprise an array of nucleic acid sequences of fragments of about 15 nucleotides in length and the sequences complementary thereto, or a fragment thereof, the fragment comprising at least about 8 consecutive nucleotides, preferably 10, 15, 20, more preferably 25, 30, 40, 47, or 50 consecutive nucleotides and containing a polymorphic base. In some embodiments the polymorphic base is within 5, 4, 3, 2, or 1 nucleotides from the center of the polynucleotide, more preferably at the center of the polynucleotide. In other embodiments, the chip may comprise an array containing any number of polynucleotides of the present invention.

An oligonucleotide may be synthesized on the surface of the substrate by using a chemical coupling procedure and an ink jet application apparatus, as described in PCT application WO95/251116 (Baldeschwieler et al.). In another aspect, a “gridded” array analogous to a dot (or slot) blot may be used to arrange and link cDNA fragments or oligonucleotides to the surface of a substrate using a vacuum system, thermal, UV, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536, 6144 or more oligonucleotides, or any other number which lends itself to the efficient use of commercially available instrumentation.

Using such arrays, the present invention provides methods of identifying the SNPs of the present invention in a sample. Such methods comprise incubating a test sample with an array comprising one or more oligonucleotide probes corresponding to at least one SNP position of the present invention, and assaying for binding of a nucleic acid from the test sample with one or more of the oligonucleotide probes. Such assays will typically involve arrays comprising oligonucleotide probes corresponding to many SNP positions and/or allelic variants of those SNP positions, at least one of which is a SNP of the present invention.

Conditions for incubating a nucleic acid molecule with a test sample vary. Incubation conditions depend on the format employed in the assay, the detection methods employed, and the type and nature of the nucleic acid molecule used in the assay. One skilled in the art will recognize that any one of the commonly available hybridization, amplification or array assay formats can readily be adapted to employ the novel SNPs disclosed herein. Examples of such assays can be found in Chard, T, An Introduction to Radioimmunoassay and Related Techniques, Elsevier Science Publishers, Amsterdam, The Netherlands (1986); Bullock, G. R. et al., Techniques in Immunocytochemistry, Academic Press, Orlando, Fla. Vol. 1 (I 982), Vol. 2 (1983), Vol. 3 (1985); Tijssen, P., Practice and Theory of Enzyme Immunoassays: Laboratory Techniques in Biochemistry and Molecular Biology, Elsevier Science Publishers, Amsterdam, The Netherlands (1985).

The samples of the present invention include, but are not limited to, nucleic acid extracts, cells, and protein or membrane extracts from cells, which may be obtained from any bodily fluids (such as blood, urine, saliva, phlegm, gastric juices, etc.), cultured cells, biopsies, or other tissue preparations. The test sample used in the above-described methods will vary based on the assay format, nature of the detection method and the tissues, cells or extracts used as the sample to be assayed. Methods of preparing nucleic acid, protein, or cell extracts are well known in the art and can be readily be adapted in order to obtain a sample that is compatible with the system utilized.

Multicomponent integrated systems may also be used to analyze SNPs. Such systems miniaturize and compartmentalize processes such as PCR and capillary electrophoresis reactions in a single functional device. An example of such technique is disclosed in U.S. Pat. No. 5,589,136, which describes the integration of PCR amplification and capillary electrophoresis in chips.

Integrated systems can be envisaged mainly when micro-fluidic systems are used. These systems comprise a pattern of micro-channels designed onto a glass, silicon, quartz, or plastic wafer included on a microchip. The movements of the samples are controlled by electric, electro-osmotic or hydrostatic forces applied across different areas of the microchip to create functional microscopic valves and pumps with no moving parts. Varying the voltage controls the liquid flow at intersections between the micro-machined channels and changes the liquid flow rate for pumping across different sections of the microchip.

For genotyping SNPs, the microfluidic system may integrate, for example, nucleic acid amplification, mini-sequencing primer extension, capillary electrophoresis, and a detection method such as laser induced fluorescence detection.

In a first step, the DNA samples are amplified, preferably by PCR. Then, the amplification products are subjected to automated mini-sequencing reactions using ddNTPs (specific fluorescence for each ddNTP) and the appropriate oligonucleotide mini-sequencing primers which hybridize just upstream of the targeted polymorphic base. Once the extension at the 3′ end is completed, the primers are separated from the unincorporated fluorescent ddNTPs by capillary electrophoresis. The separation medium used in capillary electrophoresis can be, for example, polyacrylamide, polyethyleneglycol or dextran. The incorporated ddNTPs in the single nucleotide primer extension products are identified by laser-induced fluorescence detection. This microchip can be used to process at least 96 to 384 samples, or more, in parallel.

3.7 Extension Based Techniques for the Detection of Polymorphisms

Extension based techniques for detecting polymorphisms within a nucleotide sequence can include, but are not restricted to allele-specific amplification, also known as the amplification refractory mutation system (ARMS) as disclosed in European Patent Application Publication No. 0332435 and in Newton et al., (1989, Nucl. Acids Res. 17: 2503-2516), and cloning of polymorphisms (COPS) as contemplated by Gibbs et al., (1989, Nucleic Acids Research, 17: 2347).

The extension based technique, ARMS, uses allele specific oligonucleotide (ASO) PCR primers for genotyping. In this approach, one of the two oligonucleotide primers used for PCR is designed to bind to the mutation site, most commonly with the 3′ end of the primer targeting the mutation site. Under carefully controlled conditions (annealing temperature, magnesium concentration etc.), amplification only takes place if the nucleotide at the 3′ end of the PCR primer is complementary to the base at the mutation site, with a mismatch being “refractory” to amplification. If the 3′ end of the primer is designed to be complementary to the normal gene, then PCR products should be formed when amplifying the normal gene but not genes with the mutation, and vice versa. There are numerous variations of the approach, for example, one of the simplest embodiments comprises where two amplifications are carried out, one using a primer specific for the normal gene, and a second using a primer specific for the mutant gene. This is followed by gel electrophoresis and ethidium bromide staining to detect the presence of amplified products.

A variation of the ARMS approach, termed mutagenically separated PCR (MS-PCR), comprises two ARMS primers of different lengths, one specific for the normal gene and one for the mutation. This method yields PCR products of different lengths for the normal and mutant alleles. Subsequent gel electrophoresis shows at least one of the two allelic products.

In some embodiments, Cloning of polymorphisms (COPs) can be applicable to the isolation of SNPs from particular regions of the genome, e.g., CpG islands, chromosomal bands, YACs or PAC contigs.ALEX. For example, Li et al., (2000, Nucleic Acid Research, 28(2): e1) disclose a combination of nucleic acid sequence digestion with restriction enzymes, treatment with uracil-DNA glycosylase and mung bean nuclease, PCR amplification and purification with streptavidin magnetic beads to isolate polymorphic sequences from the genomes of two human samples.

3.8 Ligation Based Assays for Detecting Polymorphisms

Another typical method of SNP detection encompasses the oligonucleotide ligation assay. A number of approaches make use of DNA ligase, an enzyme that can join two adjacent oligonucleotides hybridized to a DNA template. The specificity of the approach comes from the requirement for a perfect match between the hybridized oligonucleotides and the DNA template at the ligation site. In the oligonucleotide ligation assay (OLA), or ligase chain reaction (LCR) assay the sequence surrounding the mutation site is first amplified, and one strand serves as a template for three ligation probes, two of these are allele specific oligonucleotides (ASO) and the third a common probe. Numerous approaches can be used for the detection of the ligated products. For example, the two ASOs can be differentially labeled with fluorescent or hapten labels and ligated products detected by fluorimetric or colorimetric enzyme-linked immunosorbent assays, respectively. For electrophoresis-based systems, use of mobility modifier tags or variation in probe lengths coupled with fluorescence detection enables the multiplex genotyping of several single nucleotide substitutions in a single tube. When used on arrays, ASOs can be spotted at specific locations or addresses on a chip. PCR amplified DNA can then be added and ligation to labeled oligonucleotides at specific addresses on the array can be measured.

3.9 Signal Generating Polymorphism Detection Assays

In some embodiments, fluorescence resonance energy transfer (FRET) is contemplated as a method to identify a polymorphism within any one or more of the AS marker sequences of the present invention. FRET occurs due to the interaction between the electronic excited states of two dye molecules. The excitation is transferred from one (the donor) dye molecule to the other (the acceptor) dye molecule without emission of a photon. This is distance-dependent, that is the donor and the acceptor dye must be in close proximity. The hybridization probe system consists of two oligonucleotides labeled with fluorescent dyes. The hybridization probe pair is designed to hybridize to adjacent regions on the target DNA. Each probe is labeled with a different marker dye. Interaction of the two dyes can only occur when both are bound to their target. The donor probe is labeled with fluorophore at the 3′ end and the acceptor probe at the 5′ end. During PCR, the two different oligonucleotides hybridize to adjacent regions of the target DNA such that the fluorophores, which are coupled to the oligonucleotides, are in close proximity in the hybrid structure. The donor fluorophore (F1) is excited by an external light source, and then passes part of its excitation energy to the adjacent acceptor fluorophore (F2). The excited acceptor fluorophore (F2) emits light at a different wavelength which can then be detected and measured for molecular proximity.

In other embodiments, the MagSNiPer method, based on single base extension, magnetic separation, and chemiluminescence provides a further method for SNP identification in a nucleotide sequence. Single base nucleotide extension reaction is performed with a biotinylated primer whose 3′ terminus is contiguous to the SNP site with a tag-labeled ddNTP. Then the primers are captured by magnetic-coated beads with streptavidin, and unincorporated labeled ddNTP is removed by magnetic separation. The magnetic beads are incubated with anti-tag antibody conjugated with alkaline phosphatase. After the removal of excess conjugates by magnetic separation, SNP typing is performed by measuring chemiluminescence. The incorporation of labeled ddNTP is monitored by chemiluminescence induced by alkaline phosphatase.

In some embodiments, fluorescence polarization provides a method for identifying polymorphisms within a nucleotide sequence. For example, amplified DNA containing a polymorphic is incubated with oligonucleotide primers (designed to hybridize to the DNA template adjacent to the polymorphic site) in the presence of allele-specific dye-labeled dideoxyribonucleoside triphosphates and a commercially available modified Taq DNA polymerase. The primer is extended by the dye-terminator specific for the allele present on the template, increasing approximately 10-fold the molecular weight of the fluorophore. At the end of the reaction, the fluorescence polarization of the two dye-terminators in the reaction mixture are analyzed directly without separation or purification. This homogeneous DNA diagnostic method is shown to be highly sensitive and specific and is suitable for automated genotyping of large number of samples.

In other embodiments, surface enhanced Raman scattering can be used as a method for detecting and identifying single base differences in double stranded DNA fragments. Chumanov, G. “Surface Enhanced Raman Scattering (SERS) for Discovering and Scoring Single Based Differences in DNA” Proc. Volume SPIE, 3608 (1999). SERS has also been used for single molecule detection. Kneipp, K, (1997, Physical Review Letters, 78(9): 1667-1670). SERS results in strongly increased Raman signals from molecules which have been attached to nanometer sized metallic structures.

Illustrative examples include a genotyping method discussed by Xiao and Kwok (2003, Genome Research, 13(5): 932-939) based on a primer extension assay with fluorescence quenching as the detection. The template-directed dye-terminator incorporation with fluorescence quenching detection (FQ-TDI) assay is based on the observation that the intensity of fluorescent dye R110- and R6G-labeled acycloterminators is universally quenched once they are incorporated onto a DNA oligonucleotide primer. By comparing the rate of fluorescence quenching of the two allelic dyes in real time, the frequency of SNPs in DNA samples can be measured. The kinetic FQ-TDI assay is highly accurate and reproducible both in genotyping and in allele frequency estimation.

### 4. Vectors

Described herein are systems of vectors and host cells that can be used for the expression of at least a portion of an AS marker sequence of the present invention. A variety of expression vectors may be used in the present invention which include, but are not limited to, plasmids, cosmids, phage, phagemids, or modified viruses. Typically, such expression vectors comprise a functional origin of replication for propagation of the vector in an appropriate host cell, one or more restriction endonuclease sites for insertion of the AS marker sequence, and one or more selection markers. The expression vector can be used with a compatible host cell which may be derived from a prokaryotic or a eukaryotic organism including but not limited to bacteria, yeasts, insects, mammals, and humans.

Where the AS markers of the present invention contain transcribable sequences, those sequences in whole or in part are suitably rendered expressible in a host cell by operably linking them with a regulatory polynucleotide. The synthetic construct or vector thus produced may be introduced firstly into an organism or part thereof before subsequent expression of the construct in a particular cell or tissue type. Any suitable organism is contemplated by the invention, which may include unicellular as well as multi-cellular organisms. Suitable unicellular organisms include bacteria. Exemplary multi-cellular organisms include yeast, mammals and plants.

The construction of the vector may be carried out by any suitable technique as for example described in the relevant sections of Ausubel et al., (supra) and Sambrook et al., (“Molecular Cloning. A Laboratory Manual”, Cold Spring Harbour Press, 1989). However, it should be noted that the present invention is not dependent on and not directed to any one particular technique for constructing the vector.

Regulatory polynucleotides which may be utilised to regulate expression of the polynucleotide include, but are not limited to, a promoter, an enhancer, and a transcriptional terminator. Such regulatory sequences are well known to those of skill in the art. Suitable promoters that may be utilised to induce expression of the polynucleotides of the invention include constitutive promoters and inducible promoters.

### 5. Amino Acid Polymorphism Screening Techniques

As described above, where the particular nucleotide occurrence of a SNP is such that the nucleotide occurrence results in an amino acid change in the encoded polypeptide, the nucleotide occurrence can be identified indirectly by detecting a particular mutation or variation in the sequence of the polypeptide. For example, the ARTS-1 and TCOF1 polymorphisms listed in Table 1 and an IL-23R polymorphism disclosed in Table 2 each comprise a non-synonymous base substitution in the coding region of the corresponding gene, which causes a change in the amino acid sequence. In representative examples, the presence of G at rs11209026 within the IL-23R coding region changes the amino acid residue at position 381 of the IL-23R amino acid sequence (as set forth for example in GenPept Accession No. NP_{—}653302 [GI:24430212] or SEQ ID NO: 4 WO 2008/144827) from Gln to Arg. In other representative examples relating to the ARTS-1 coding sequence (as set forth for example in SEQ ID NO: 31), the presence of G instead of C at rs27044 changes the corresponding amino acid residue at residue 730 of the ARTS-1 polypeptide (as set forth for example in GenPept Accession No. NP_{—}057526 [GI:94818901] or SEQ ID NO: 2 of WO 2008/144827) from Glu to Gln; or the presence of C instead of T at rs17482078 changes the corresponding amino acid residue at residue 725 of the ARTS-1 polypeptide from Gln to arginine Arg; or the presence of C instead of T at rs10050860 changes the corresponding amino acid residue at residue 575 of the ARTS-1 polypeptide from Asn to Asp; or the presence of T instead of C at rs2287987 changes the corresponding amino acid residue at residue 349 of the ARTS-1 polypeptide from Val to Met; the presence of T instead of C at rs30187 changes the corresponding amino acid reside at residue 528 of the ARTS-1 polypeptide from Arg to lysine Lys. In still other representative examples, the presence of C instead of T at rs15251 changes the corresponding amino acid residue at residue 1313 of the TCOF1 polypeptide from Val to Ala. Accordingly, in some embodiments, the sample is analyzed for the presence of Val at residue 1313 of the TCOF1 polypeptide, which indicates that the subject has AS or is at risk of developing AS. Accordingly, the presence or absence of a change in the amino acid sequence of a polypeptide can be analyzed by any method known in the art, not restricted to direct sequencing, protein truncation tests and protein migration analysis for diagnosing the presence or risk of development of AS.

5.1 Protein Truncation Assay (PTT)

In some embodiments, the PTT can be used to identify polymorphisms within a protein sequence. PTT uses in vitro transcription and translation of the cDNA generated to focus on mutations that generate proteins with an altered size; shorter proteins caused by premature translation termination. For some genes containing large exons, PTT can also be performed using a genomic DNA target (Hogervorst, F. B. L., 1997, Promega Notes Magazine, 62: 7-11).

Thus, in the above embodiment, the coding region of a gene is screened for the presence of translation terminating mutations using de novo protein synthesis from the amplified copy. The procedure includes three important steps. The first step involves the isolation of genomic DNA and amplification of the target gene coding sequences using PCR or, alternatively, isolation of RNA and amplification of the target sequence using Reverse Transcription PCR (RT-PCR). The resulting PCR products are then used as a template for the in vitro synthesis of RNA, which is subsequently translated into protein. The final step is the SDS-PAGE analysis of the synthesized protein. The shorter protein products of mutated alleles are easily distinguished from the full length protein products of normal alleles.

Mutant truncated proteins can result from for example, nonsense substitution mutations, frameshift mutations, in-frame deletions, and splice site mutations.

For example, a nonsense substitution mutation occurs when a nucleotide substitution causes a codon that normally encodes an amino acid to code for one of the three stop signals (TGA, TTA, TAG). For such mutations, the protein truncation point occurs at the corresponding position in the gene at which the mutation occurs.

Frameshift mutations result from the addition or deletion of any number of bases that is not a multiple of three (e.g., one or two base insertion or deletion). For such frameshift mutations, the reading frame is altered from the point of mutation downstream. A stop codon, and resulting truncation of the corresponding encoded protein product, can occur at any point from the position of the mutation downstream.

In-frame deletions result from the deletion of one or more codons from the coding sequence. The resulting protein product lacks only those amino acids that were encoded by the deleted codons.

Splice site mutations result in an improper excision and/or joining of exons. These mutations can result in inclusion of some or all of an intron in the mRNA, or deletion of some or all of an exon from the mRNA. In some instances, these insertions or deletions result in stop codon being encountered prematurely, as typically occurs with frameshift mutations. In other instances, one or more specific exons are deleted from the mature mRNA in such a manner that the proper reading frame is maintained for the remaining exons, i.e., non-contiguous exons are fused in frame with each other. For such splice mutations, the encoded protein may terminate at the appropriate stop codon, but is shortened by the absence of the un-spliced internal exon.

5.2 Protein Sequencing

In some embodiments, sequencing of a polypeptide may be performed by site-directed or random cleavage of the polypeptide using, for example endopeptidases or CNBr, to produce a set of polypeptide fragments and subsequent sequencing of the polypeptide fragments by, for example, Edman sequencing or mass spectrometry, as is known in the art. Alternatively, the polypeptide probes or polypeptide fragments could be sequenced by use of antibody probes as for example described by Fodor et al in U.S. Pat. No. 5,871,928. Briefly, such antibody probes specifically recognise particular subsequences (e.g., at least three contiguous amino acids) found on a polypeptide. Optimally, these antibodies would not recognise any sequences other than the specific desired subsequence and the binding affinity should be insensitive to flanking or remote sequences found on a target molecule.

The Edman degradation process is commonly used, while other methods have been developed and can be used in certain instances. In the Edman degradation method, amino acid removal from the end of the protein is accomplished by reacting the N-terminal amino acid residue with a reagent which allows selective removal of that residue from the protein. The resulting amino acid derivative is converted into a stable compound which can be chemically removed from the reaction mixture and identified.

Most current chemical sequencing methods are done with an amount of protein in the 5-100 nm range. It has been reported that micro-sequencing of polypeptides by reverse phase high pressure liquid chromatography using ultraviolet light detection means has been accomplished with protein samples in the range of 50-500 pm. Other methods used in the micro-sequencing of polypeptides involves radio labeling of the peptide or reagent, intrinsic radio labeling of the polypeptide, and enhanced UV detection of sequence degradation products, and others.

It is possible to determine the C-terminus sequence of peptides and proteins using a combination of Matrix-Assisted Laser Desorption/Ionization-Time Of Flight-Mass Spectrometry (MALDI-TOF-MS) and enzymatic digestions using for example, the Applied Biosystems Sequazyme technology. In some illustrative examples, Carboxypeptidase Y is a non-specific exoprotease, which sequentially cleaves all residues, including proline, from the C-terminus. This generates a nested set of fragments that form a sequence “ladder.” The masses of individual members of the set are determined by MALDI-TOF-MS, and the amino acids are identified from the unique mass differences between peaks. Trace quantities of peptides and proteins, as little as 2 pmol, can be analyzed. Up to 20 residues can be identified in less than 30 minutes. Aminopeptidase can similarly be used to generate N-terminal ladders from the peptides.

In some embodiments, peptides can be fragmented by either post-source decay (PSD) or collision-induced dissociation (CID) for use in MS/MS studies. The process of PSD starts as the peptide is ionized using a higher than normal laser power to pump more energy into the peptide. PSD is also facilitated by the selection of a matrix that is more favorable to promoting fragmentation. The ionized peptides are extracted from the ion source and gain full kinetic energy necessary for mass analysis. As the ions travel down the flight tube, those having excess internal energy must change. If enough energy is localized in a single bond, it will break apart, producing a product ion and a neutral fragment. Product ions come in many forms which can include N-terminal, C-terminal, and internal fragments. The ion reflector separates ions based on their kinetic energy. When ions enter the reflector, they experience an electric field that reverses their direction. The product ions have kinetic energies that are directly proportional to the ratio between the product ion mass and the peptide precursor mass. For low mass product ions, those having low kinetic energy, the reflection shortens their flight path, reducing the time required to reach the detector. For higher mass ions, those having a higher kinetic energy, reflection lengthens their flight path, increasing the time of flight to the detector. Modulation of the potential applied to the ion reflector enables collection of high quality PSD spectra with good mass accuracy.

In CID, the peptide ion interacts with a collision gas to modulate the internal energy and promote fragmentation. As with PSD, fragmentation does not change the velocity of the ions once they are in the flight tube, so the peptide precursor ion and product ions only separate when they encounter the ion reflector.

5.3 Immunohistology

In some embodiments immunohistochemical analysis of a tissue sample from a subject suspected of having AS or being at risk of developing AS can be employed to detect the presence of a related sequence polymorphism. For examples, antibodies specific to the region of the protein sequence suspected of containing the polymorphism can be raised and used in a visual test to identify polymorphisms. Specifically, tissue samples can be probed with an antibody of choice before detecting the level of bound antibody and comparing it with a control sample. To enhance visual detection, the secondary antibody can be conjugated with a fluorophore such as Texas Red.

5.4 Immunoassays

5.4.1 Antigen-Binding Molecules

The invention also contemplates antigen-binding molecules that bind specifically to the polypeptide encoded by any of the AS markers of the invention or to a fragment of said polypeptide. For example, the antigen-binding molecules may comprise whole polyclonal antibodies. Such antibodies may be prepared, for example, by injecting a polypeptide of the invention or fragment thereof into a production species, which may include mice or rabbits, to obtain polyclonal antisera. Methods of producing polyclonal antibodies are well known to those skilled in the art. Exemplary protocols which may be used are described for example in Coligan et al., 1991, Current Protocols in Immunology, (John Wiley & Sons, Inc) and Ausubel et al., (1994-1998, supra), in particular Section III of Chapter 11.

In lieu of the polyclonal antisera obtained in the production species, monoclonal antibodies may be produced using the standard method as described, for example, by Köhler and Milstein (1975, Nature 256, 495-497), or by more recent modifications thereof as described, for example, in Coligan et al., (1991, supra) by immortalizing spleen or other antibody producing cells derived from a production species which has been inoculated with a polypeptide of the invention or a fragment thereof.

The invention also contemplates as antigen-binding molecules Fv, Fab, Fab′ and F(ab′)₂ immunoglobulin fragments. Alternatively, the antigen-binding molecule may comprise a synthetic stabilised Fv fragment. Exemplary fragments of this type include single chain Fv fragments (sFv, frequently termed scFv) in which a peptide linker is used to bridge the N terminus or C terminus of a V_{H} domain with the C terminus or N-terminus, respectively, of a V_{L} domain. ScFv lack all constant parts of whole antibodies and are not able to activate complement. Suitable peptide linkers for joining the V_{H} and V_{L} domains are those which allow the V_{H} and V_{L} domains to fold into a single polypeptide chain having an antigen binding site with a three dimensional structure similar to that of the antigen binding site of a whole antibody from which the Fv fragment is derived. Linkers having the desired properties may be obtained by the method disclosed in U.S. Pat. No. 4,946,778. However, in some cases a linker is absent. ScFvs may be prepared, for example, in accordance with methods outlined in Kreber et al., (1997, J. Immunol. Methods; 201(1): 35-55). Alternatively, they may be prepared by methods described in U.S. Pat. No. 5,091,513, European Patent No 239,400 or the articles by Winter and Milstein (1991, Nature 349:293) and Plünckthun et al., (1996, In Antibody engineering: A practical approach. 203-252).

Alternatively, the synthetic stabilised Fv fragment comprises a disulphide stabilised Fv (dsFv) in which cysteine residues are introduced into the V_{H} and V_{L} domains such that in the fully folded Fv molecule the two residues will form a disulphide bond there between. Suitable methods of producing dsFv are described for example in (Glockscuther et al., Biochem. 29: 1363-1367; Reiter et al., 1994, J. Biol. Chem. 269: 18327-18331; Reiter et al., 1994, Biochem. 33: 5451-5459; Reiter et al., 1994. Cancer Res. 54: 2714-2718; and Webber et al., 1995, Mol. Immunol. 32: 249-258).

Also contemplated as antigen-binding molecules are single variable region domains (termed dAbs) as for example disclosed in (Ward et al., 1989, Nature 341: 544-546; Hamers-Casterman et al., 1993, Nature. 363: 446-448; and Davies & Riechmann, 1994, FEBS Lett. 339: 285-290).

Alternatively, the antigen-binding molecule may comprise a “minibody”. In this regard, minibodies are small versions of whole antibodies, which encode in a single chain the essential elements of a whole antibody. Suitably, the minibody is comprised of the V_{H} and V_{L} domains of a native antibody fused to the hinge region and CH3 domain of the immunoglobulin molecule as, for example, disclosed in U.S. Pat. No. 5,837,821.

In an alternate embodiment, the antigen binding molecule may comprise non-immunoglobulin derived, protein frameworks. For example, reference may be made to (Ku & Schultz, 1995, Proc. Natl. Acad. Sci. USA, 92: 652-6556) which discloses a four-helix bundle protein cytochrome b562 having two loops randomised to create complementarity determining regions (CDRs), which have been selected for antigen binding.

The antigen-binding molecule may be multivalent (i.e., having more than one antigen-binding site). Such multivalent molecules may be specific for one or more antigens. Multivalent molecules of this type may be prepared by dimerisation of two antibody fragments through a cysteinyl-containing peptide as, for example disclosed by (Adams et al., 1993, Cancer Res. 53: 4026-4034; Cumber et al., 1992, J. Immunol. 149: 120-126). Alternatively, dimerisation may be facilitated by fusion of the antibody fragments to amphiphilic helices that naturally dimerise (Pack P. Plünckthun, 1992, Biochem. 31: 1579-1584), or by use of domains (such as the leucine zippers jun and fos) that preferentially heterodimerize (Kostelny et al., 1992, J. Immunol. 148: 1547-1553). In an alternate embodiment, the multivalent molecule may comprise a multivalent single chain antibody (multi-scFv) comprising at least two scFvs linked together by a peptide linker. In this regard, non-covalently or covalently linked scFv dimers termed “diabodies” may be used. Multi-scFvs may be bispecific or greater depending on the number of scFvs employed having different antigen binding specificities. Multi-scFvs may be prepared for example by methods disclosed in U.S. Pat. No. 5,892,020.

5.5 Protein Arrays

In some embodiments, the of the invention can be detected through the use of protein arrays. Protein arrays may comprise a surface upon which are deposited at specially defined locations at least two protein moieties characterised in that the protein moieties are those of the sequence of interest. The protein moieties can be attached to the surface either directly or indirectly. The attachment can be non-specific (e.g., by physical absorption onto the surface or by formation of a non-specific covalent interaction). In some embodiments the protein moieties are attached to the surface through a common marker moiety appended to each protein moiety. In another embodiment, the protein moieties can be incorporated into a vesicle or liposome which is tethered to the surface. An example of such a protein array is described in Frank, R (2002, Comb. Chem. 5: 429-440).

In an alternate embodiment, the non-synonymous SNPs of the invention can be detected through the use of antibody arrays. In a similar manner to RNA profiling on DNA chips, antibody arrays can be employed for overlay assays to identify and quantify proteins and their specific amino acids. An illustrative example of this type is the protein binding assay, wherein an antibody array is overlayed with protein complexes and specific antibodies can detect potential binding partners of the proteins bound to the array (Wang et al., 2000, Mol. Cell. Biol, 20: 4505-4512; and Maercker, Bioscience Reports, 25(1/2): 57-70).

### 6. Polymorphism Sequence Analysis

Further contemplated by the present invention is the analysis of samples from subjects suspected or having AS or at risk of developing AS using a sequence analysis program. For example, the sequence analysis program may be in the form of a computer program for use in homology searching, mapping, haplotyping, genotyping or pharmacogenetic analysis. The information gained from the analysis can be in any computer readable format and can comprise any composition of matter used to store information or data, including, for example, floppy disks, tapes, chips, compact disks, video disks, punch cards or hard drives to name but a few.

### 7. Kits

All the essential materials and reagents required for detecting AS-associated polymorphisms in at least a portion of an AS marker sequence according to the invention may be assembled together in a kit. The kits may also optionally include appropriate reagents for detection of labels, positive and negative controls, washing solutions, blotting membranes, microtitre plates dilution buffers and the like. For example, a nucleic acid-based detection kit for the identification of polymorphisms may include (i) an AS marker polynucleotide (which may be used as a positive control), (ii) a primer or probe that specifically hybridizes to at least a portion of the IL-1R1 gene locus, the IL-1R2 gene locus, the CD74 gene locus, the 2Q31.3 and 4Q13.1 chromosome loci, the ARTS-1 gene, the IL-23R gene, the TNFR1 and TRADD gene loci and the 2P15 and 21Q22 chromosome loci sequences, and optionally one or more other AS markers, at or around the suspected SNP site. Also included may be enzymes suitable for amplifying nucleic acids including various polymerases (Reverse Transcriptase, Taq, Sequenase™ DNA ligase etc. depending on the nucleic acid amplification technique employed), deoxynucleotides and buffers to provide the necessary reaction mixture for amplification. Such kits also generally will comprise, in suitable means, distinct containers for each individual reagent and enzyme as well as for each primer or probe. The kit can also feature various devices and reagents for performing one of the assays described herein; and/or printed instructions for using the kit to identify the presence of an AS-associated polymorphism within the IL-1R1 gene locus, the IL-1R2 gene locus, the CD74 gene locus, the 2Q31.3 and 4Q13.1 chromosome loci, the ARTS-1 gene, the IL-23R gene, the TNFR1 and TRADD gene loci and the 2P15 and 21Q22 chromosome loci sequences. The kit may further contain reagents (e.g., primers, probes or antigen-binding molecules) for detecting the presence of other AS markers, illustrative examples of which include the HLA-B27 gene and its expression products.

In some embodiments, the kit may comprise appropriate agents for the detection of polymorphisms within the polypeptides encoded by the IL-1R1 gene locus, the IL-1R2 gene locus, the CD74 gene locus, the 2Q31.3 and 4Q13.1 chromosome loci, the ARTS-1 gene, the IL-23R gene, the TNFR1 and TRADD gene loci and the 2P15 and 21Q22 chromosome loci sequences (“AS marker polypeptides”) by Mass Spectrometry (MS). In illustrative examples of this type, an MS polymorphism detection kit may comprise (i) a vector that expresses an AS marker polynucleotide with at least one AS-associated polymorphism for the expression of an AS marker polypeptide in a host cell (which may be used as a positive control) (ii) enzymes for digesting the expressed polypeptide, comprising for example non-specific exoproteases; and (iii) polypeptide fragments (which may be used as positive controls). The kit can also feature various devices and reagents for performing MS or any related form of MS known in the art; and/or printed instructions for using the kit to identify the presence of an AS-associated polymorphism within the AS marker polypeptide as described, for example, above.

### 8. Methods of Managing AS

The present invention also extends to the management of AS, or prevention of further progression of AS, or assessment of the efficacy of therapies in subjects following positive diagnosis for the presence of an AS-associated polymorphism in the subjects. Generally, the management of AS often includes a treatment regime involving medication, exercise, physical therapy and if necessary surgery. Examples of effective medications include but are not restricted to nonsteroidal anti-inflammatory drugs (NSAIDS) such as Sulfasalazine (Azulfidine), Methotrexate (Rheumatrex or Trexall) and Corticosteroids (cortisone); TNF blockers such as etanercept (Enbrel), infliximab (Remicade) and adalimumab (Humira);

It will be understood, however, that the present invention encompasses the use of any agent or process that is useful for treating or preventing AS and is not limited to the aforementioned illustrative management strategies and compounds.

Typically, AS-ameliorating agents will be administered in pharmaceutical (or veterinary) compositions together with a pharmaceutically acceptable carrier and in an effective amount to achieve their intended purpose. The dose of active compounds administered to a subject should be sufficient to achieve a beneficial response in the subject over time such as a reduction in, or relief from, the symptoms of AS and the prevention of the disease from developing further. The quantity of the pharmaceutically active compounds(s) to be administered may depend on the subject to be treated inclusive of the age, sex, weight and general health condition thereof. In this regard, precise amounts of the active compound(s) for administration will depend on the judgement of the practitioner. In determining the effective amount of the active compound(s) to be administered in the treatment or prevention of AS, the physician or veterinarian may evaluate severity of any symptom associated with the presence of AS including symptoms related to AS such as for example characterized by acute, painful episodes followed by temporary periods of remission. In any event, those of skill in the art may readily determine suitable dosages of the AS-ameliorating agents and suitable treatment regimens without undue experimentation.

In order that the invention may be readily understood and put into practical effect, particular preferred embodiments will now be described by way of the following non-limiting examples.

### EXAMPLES

### Example 1

### Detection of AS-Associated Polymorphisms within the IL-1R1, IL-1R2 and CD74 Gene Loci and within the Chromosome Loci 2Q31.3 and 4Q13.1

### Patients

As part of the study, 1886 Australian, British and North American Caucasian AS cases of white European descent were enrolled, fulfilling the modified New York Criteria for the disease. Control genotypes were obtained from the Wellcome Trust case-Control Consortium study of the 1958 British Birth Cohort and from the illumine iControlDB database of North American healthy controls (n=3407). Cases were genotyped for 300,000-370,000 SNPs using Illumina HumHap300 or HumHap370 microarray genotyping slides. Cases and controls of non-white European ancestry were identified using Eigensoft principle components analysis approaches and were excluded, and related individuals identified by IBS analysis using PLINK, were excluded. Case-control analysis was then performed by Cochrane-Armitage test. Genomewide significance (GWS) was defined as P<10⁻⁷, and suggestive genomewide significance (sGWS) as P<10⁻⁵.

### Genotypina of Polymorphisms within the IL-1R1, IL-1R2 and CD74 Gene Loci and within the Chromosome Loci 2Q31.3 and 4Q13.1

Genotyping was performed using Illumina HumHap300 microarray genotyping slides as described above for all cases.

The study confirmed strong association of the MHC with AS, with a minimum p-value achieved of 10⁻²⁶⁷. Strong association was also observed within the IL-1R1 gene locus (rs949963, P=3×10⁻⁵), the IL-1R2 gene locus (rs2310173, P=9×10⁻⁵), the CD74 gene locus (rs15251, P=4×10⁻³) and the chromosome loci 2Q31.3 (rs1018326, P=2×10⁻⁶) and 4Q13.1 (rs10517820, P=2×10⁻⁵). See FIGS. 2-6 and SEQ ID NO: 1-5 for sequence information relating to the correlating SNPs and associated genetic loci.

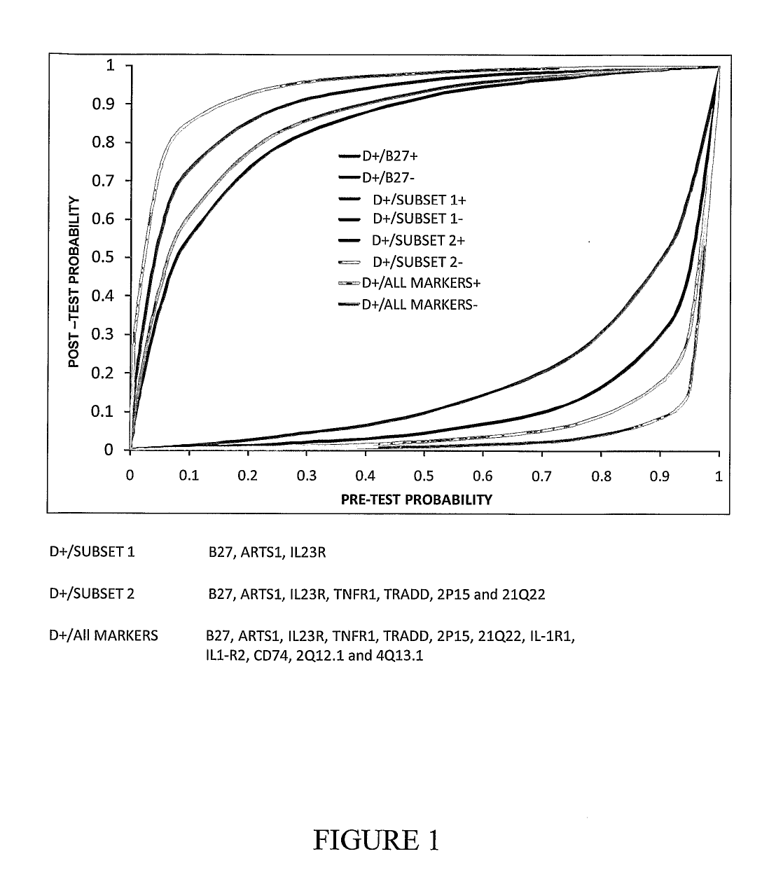

The diagnostic value of each of the AS markers was tested and the findings are presented in Table 3 below as the post-test probability of a diagnosis of AS calculated based on the pre-test probability of disease, and the genetic findings of either the AS marker B27 alone, or different combinations of AS markers selected from the B27, ARTS-1, IL-23R, TNFR1, TRADD, 2P15, 21Q22, IL-1R1, IL1-R2, CD74, 2Q31.3 and 4Q13.1 AS markers. The corresponding diagnostic value of MRI scanning, currently considered the most sensitive method for AS diagnosis is included for comparison. FIG. 1 illustrates these findings in graphical format.

TABLE 3

| | B27 Positive | | | MRI Positive | | | Subset 2* | | | ALL Markers** | | |
|---|---|---|---|---|---|---|---|---|---|---|---|---|
| PRIOR P | 0.4% | 5% | 50% | 0.4% | 5% | 50% | 0.4% | 5% | 50% | 0.4% | 5% | 50% |
| P(D+\|G1) | 4% | 37% | 92% | 3% | 31% | 90% | 8.8% | 56% | 96% | 17.3% | 73.3% | 98.1% |
| P(D+\|G0) | 0.043% | 0.56% | 10% | 0.045% | 1% | 10% | 0.009% | 0.12% | 2.3% | 0.004% | 0.05% | 1.0% |

*B27, ARTS1, IL23R, TNFR1, TRADD, 2P15 and 21Q22
**B27, ARTS1, IL23R, TNFR1, TRADD, 2P15, 21Q22, IL-1R1, IL1-R2, CD74, 2Q31.3 and 4Q13.1

### Example 2

### Detection of AS-Associated Polymorphisms within the TNFR1, 2P15, 21Q22 and TRADD Loci

### Patients

As part of the study, 2108 Australian, British and North American Caucasian AS cases of white European descent were enrolled, fulfilling the modified New York Criteria for the disease. Control genotypes were obtained from the Wellcome Trust case-Control Consortium study of the 1958 British Birth Cohort (n=1500) and from the illumine iControlDB database of North American healthy controls. Cases were genotyped for 317,000 SNPs using Illumina HumHap300 microarray genotyping slides. Cases and controls of non-white European ancestry were identified using Eigensoft principle components analysis approaches and were excluded, and related individuals identified by IBS analysis using PLINK, were excluded. Case-control analysis was then performed by Cochrane-Armitage test. Genomewide significance (GWS) was defined as P<10⁻⁷, and suggestive genomewide significance (sGWS) as P<10⁻⁵.

### Genotyping of Polymorphisms within the TNFR1, 2P15, 21Q22 and TRADD Loci

Genotyping was performed using Illumina HumHap300 microarray genotyping slides as described above for all cases.

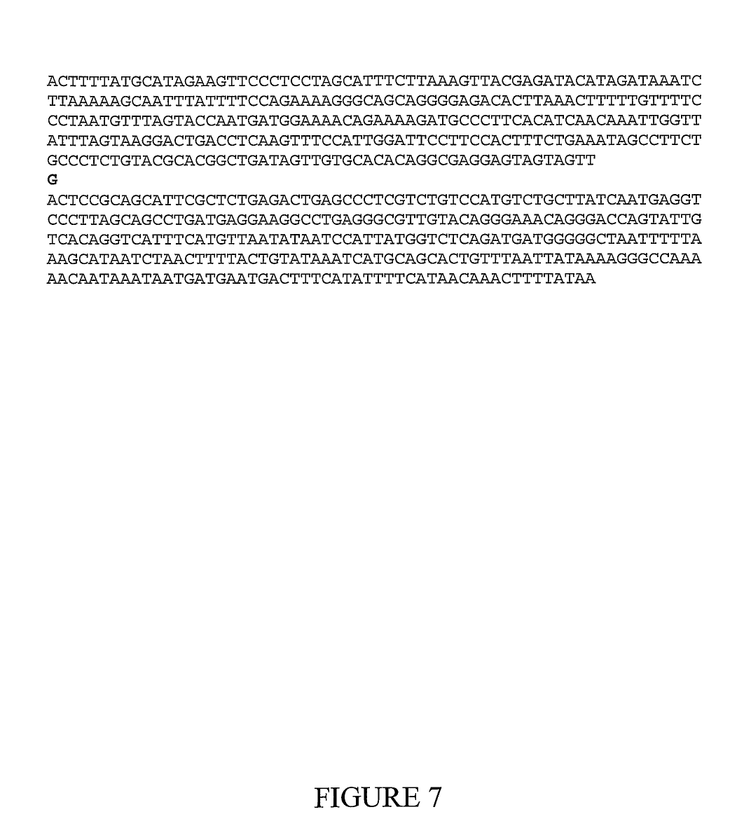

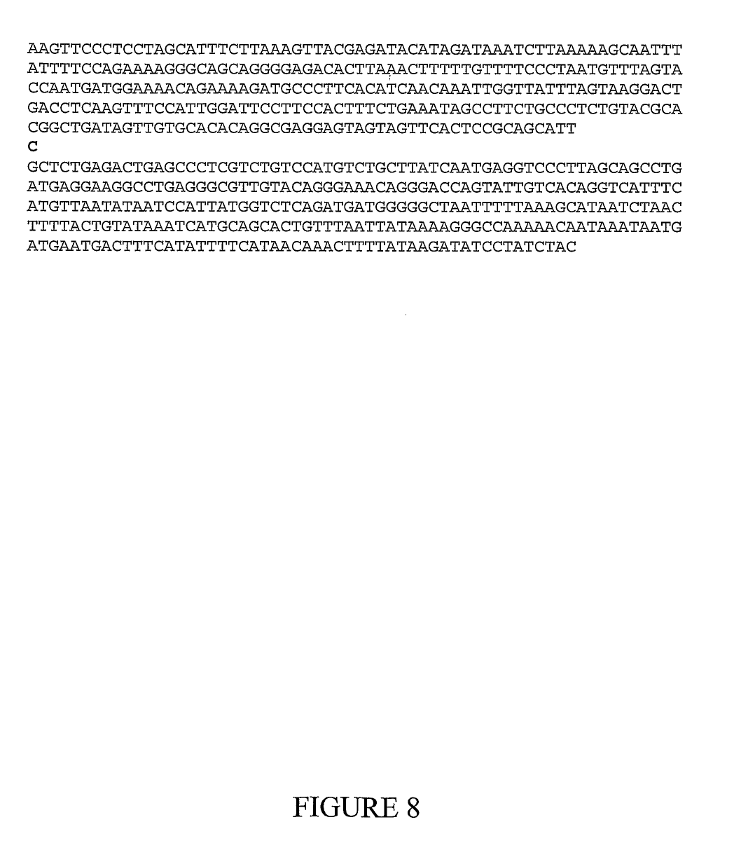

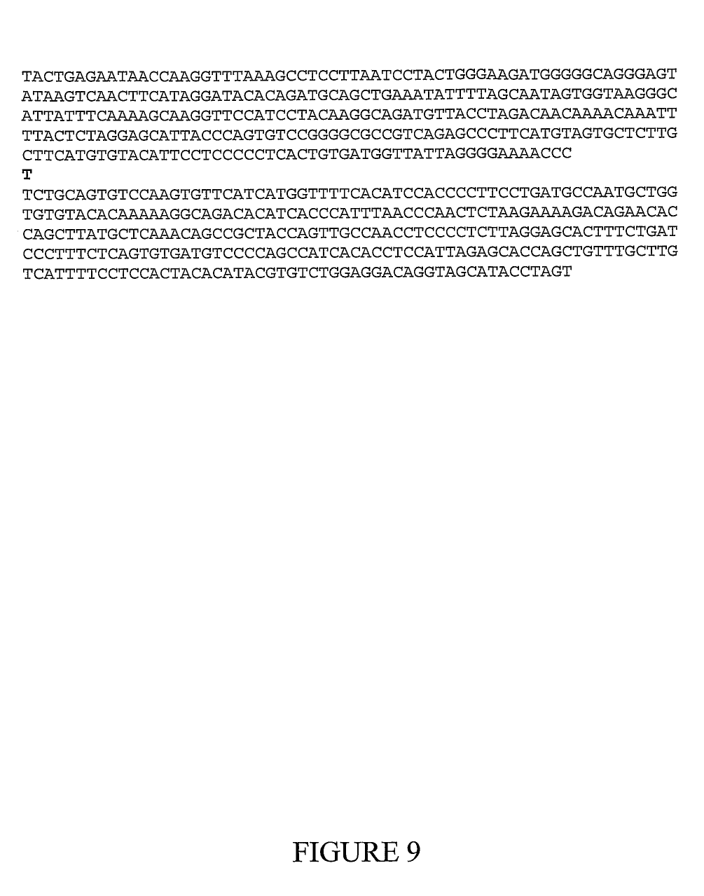

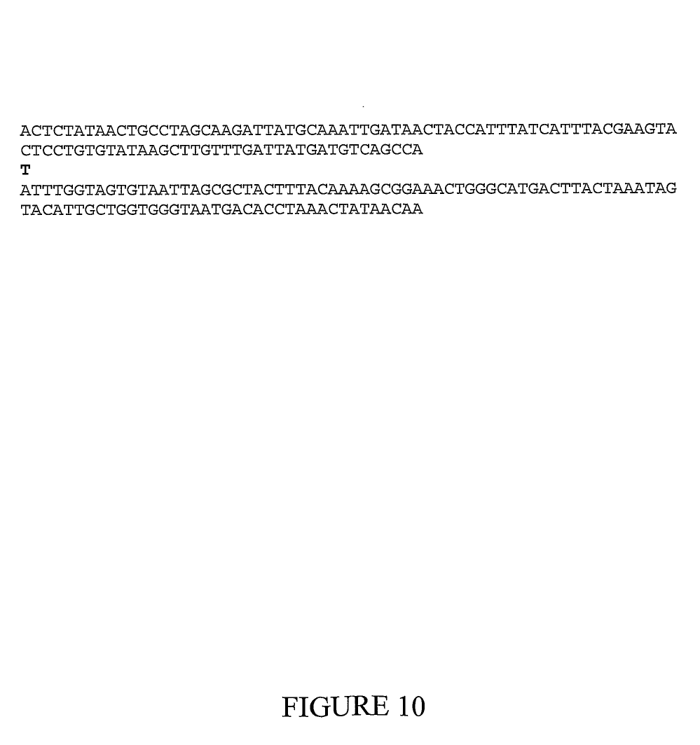

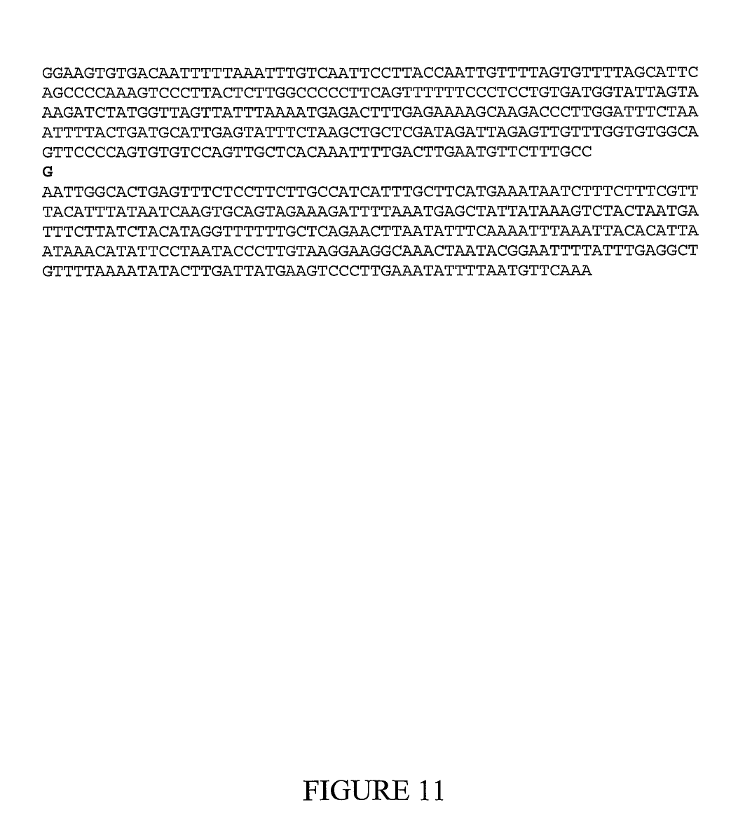

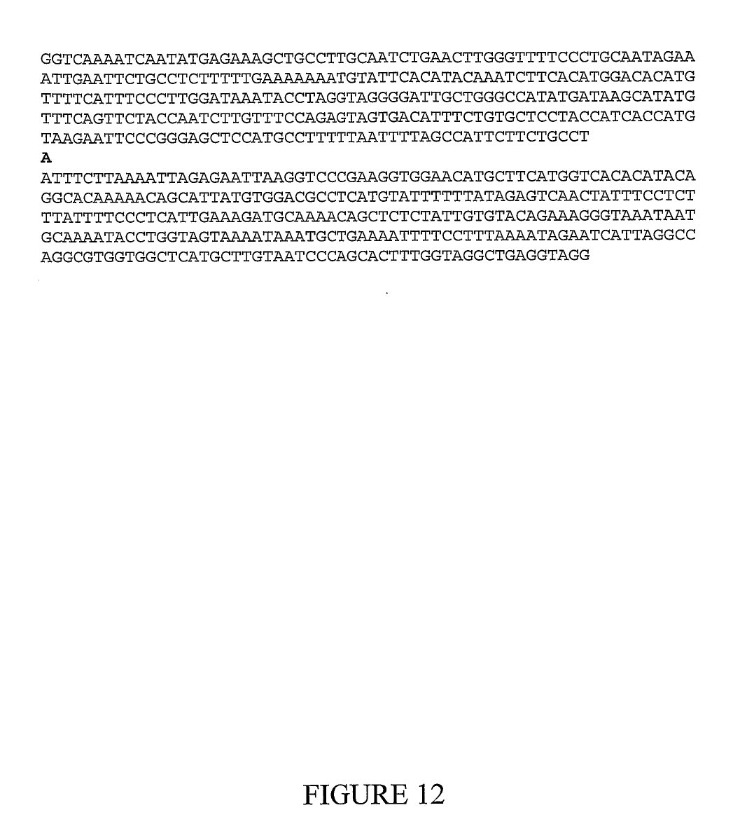

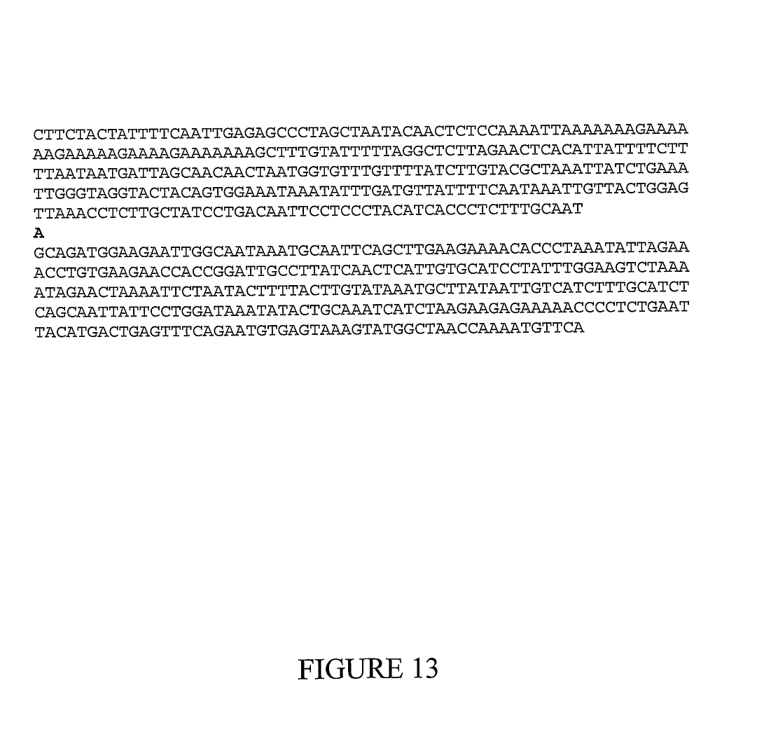

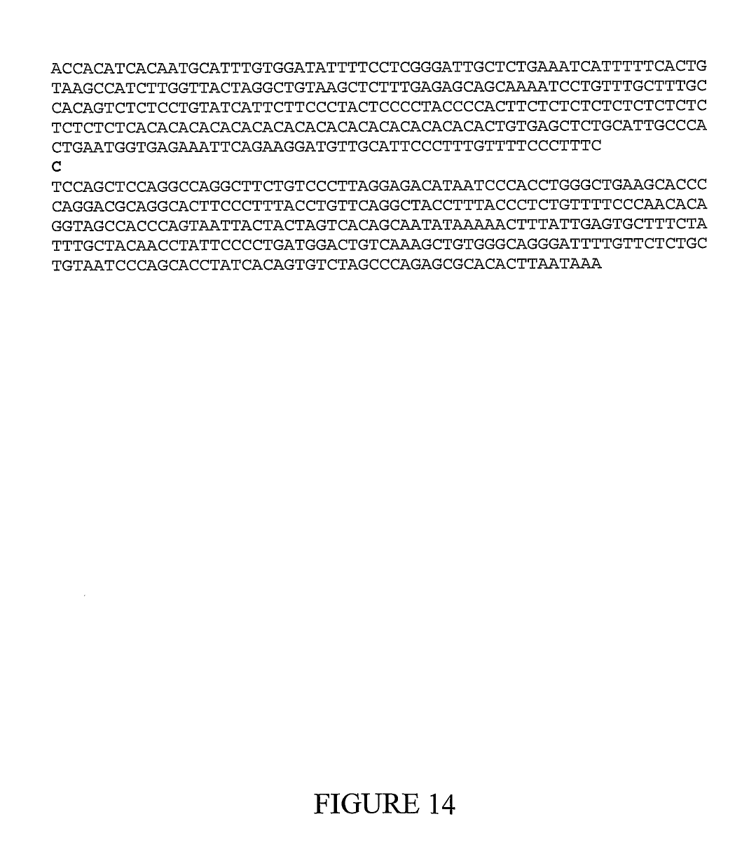

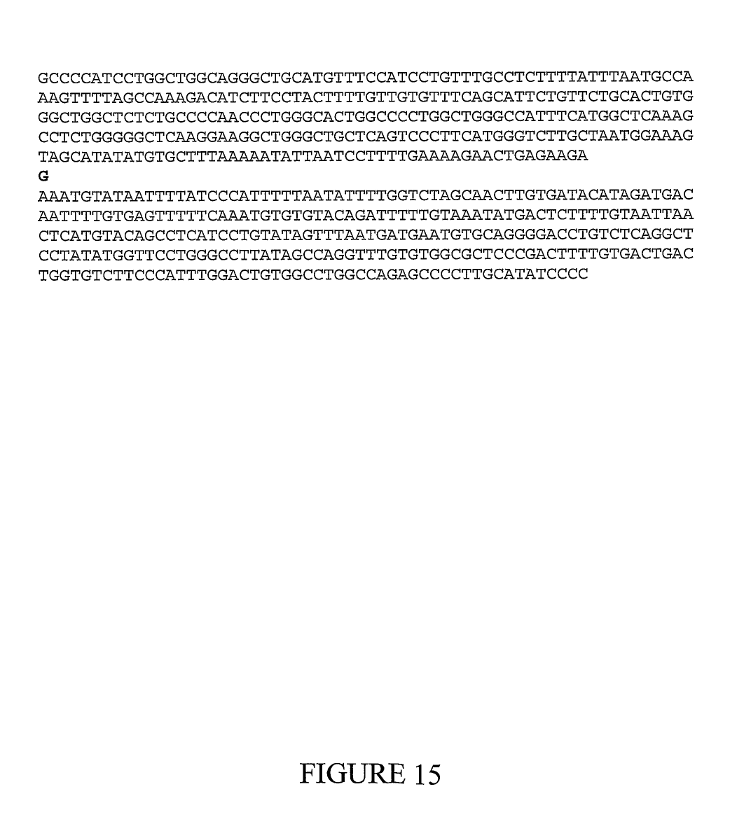

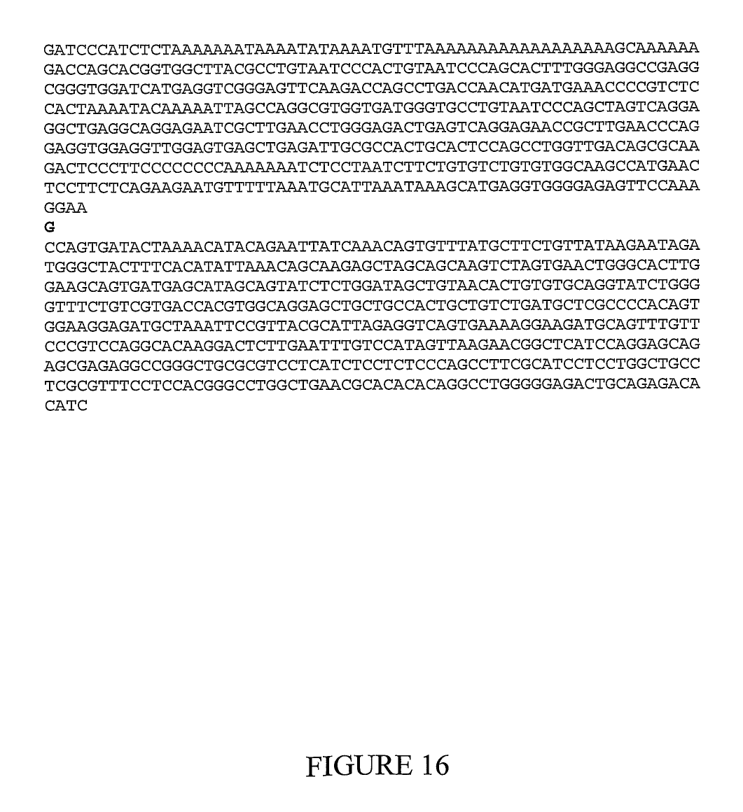

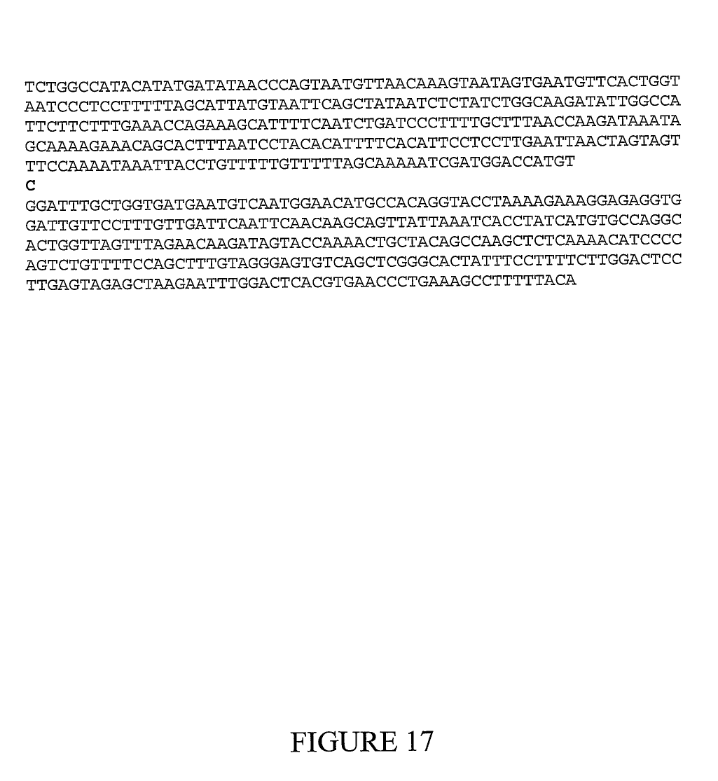

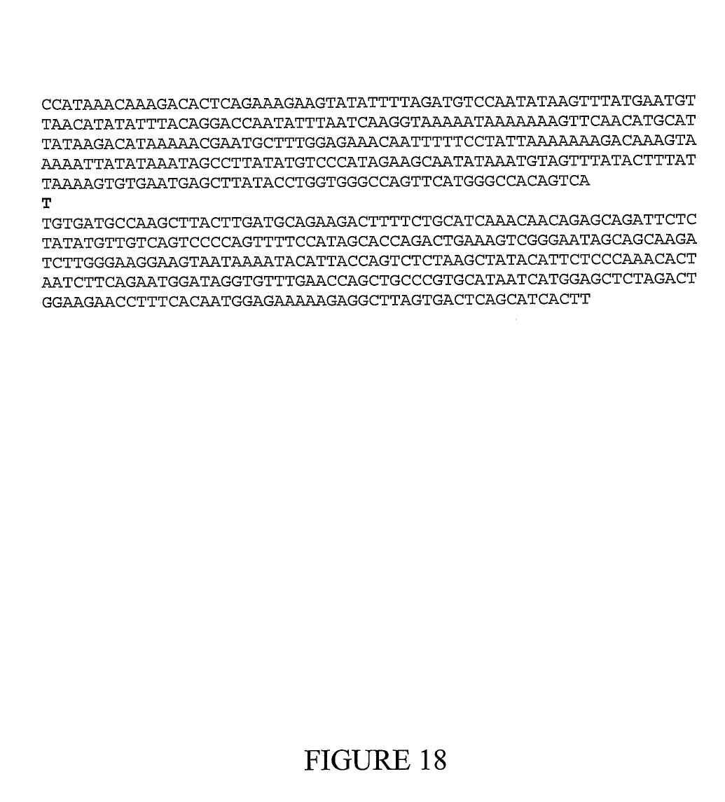

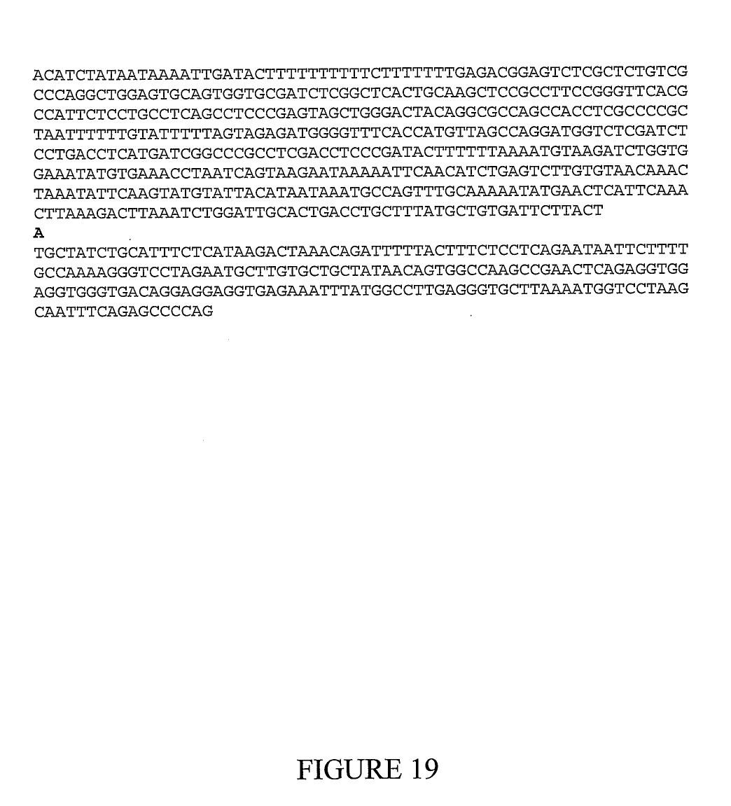

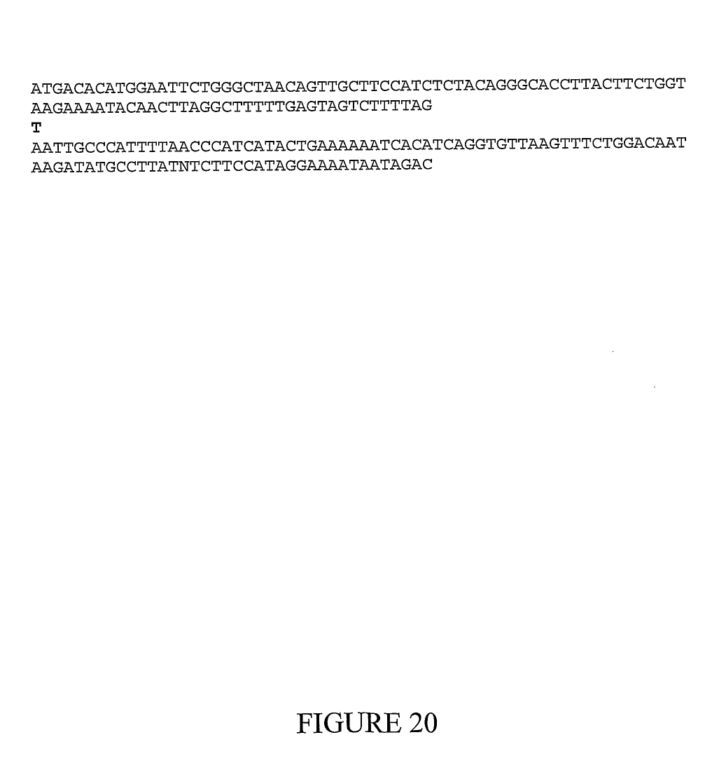

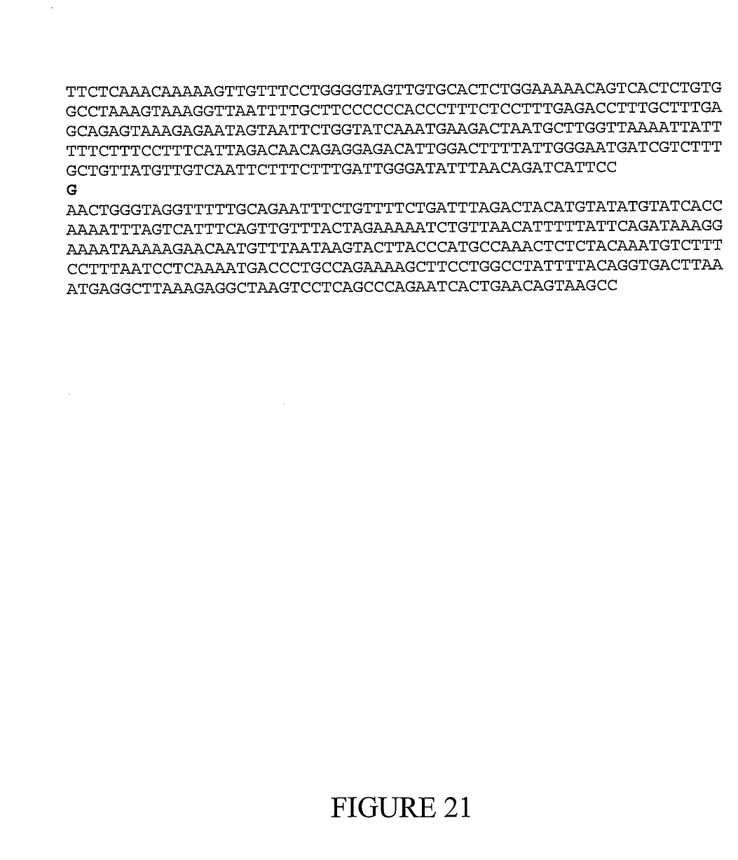

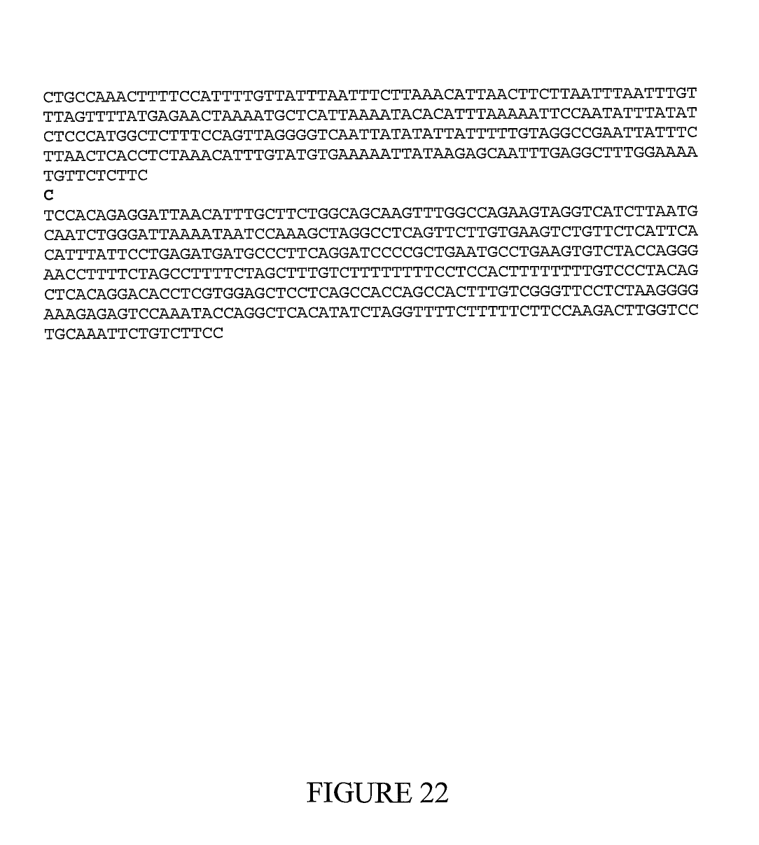

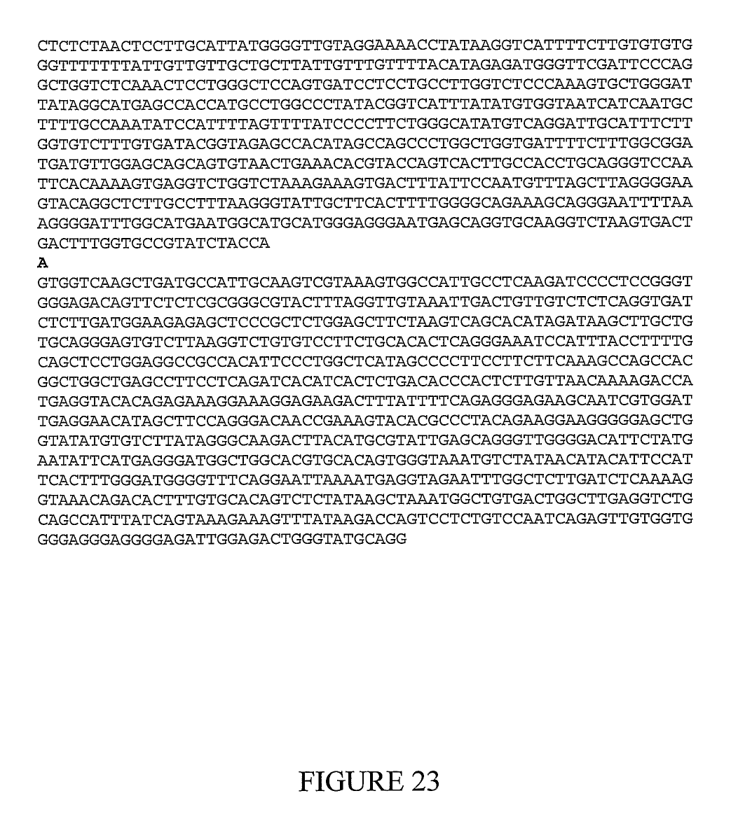

The study confirmed strong association of the MHC with AS, with a minimum p-value achieved of 10⁻²⁶⁷. Strong association was also observed within chromosome loci 21Q22 (rs2242944, P=2.6×10⁻¹⁰) and 2P15 (rs10865331, P=1.1×10⁻¹⁴). In addition strong association was observed in the TNFR1 gene locus (rs4149576, P=4.8×10⁻⁶) and the TRADD gene locus (rs9033, P=3.2×10⁻⁵); see SEQ ID NO: 6-22, FIGS. 7 to 23 and Tables 1 and 2 for sequence information relating to the corresponding SNPs and associated genetic loci. The genetic finding of the association study of genetic markers in AS-associated genes is detailed below in Table 4.

TABLE 4

| | CHROMO- | GENE/ | ODDS | | P- |
|---|---|---|---|---|---|
| MARKER | SOME | REGION | RATIO | CHI2 | VALUE |
| RS11209026 | 1 | IL23R | 0.54 | 36.54 | 1.50E−09 |
| RS10865331 | 2 | 2P15 | 1.37 | 54.62 | 1.47E−13 |
| RS30187 | 5 | ARTS1 | 1.30 | 37.38 | 9.72E−10 |
| RS4149576 | 12 | TNFR1 | 0.82 | 21.62 | 3.32E−06 |
| RS9033 | 16 | TRADD | 1.20 | 18.44 | 1.75E−05 |
| RS2242944 | 21 | 21Q22 | 0.76 | 37.83 | 7.71E−10 |

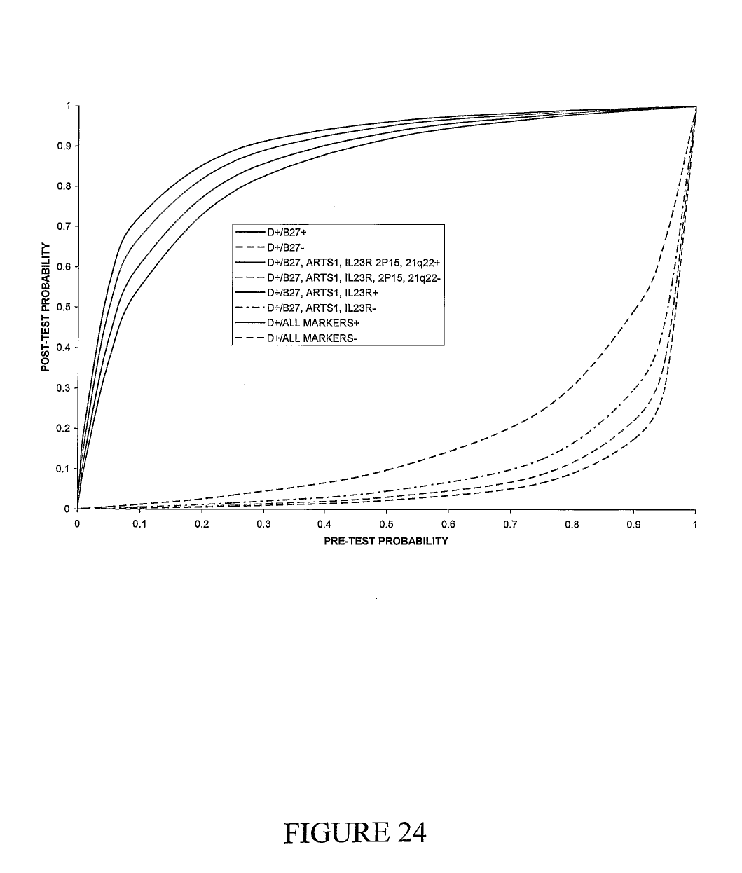

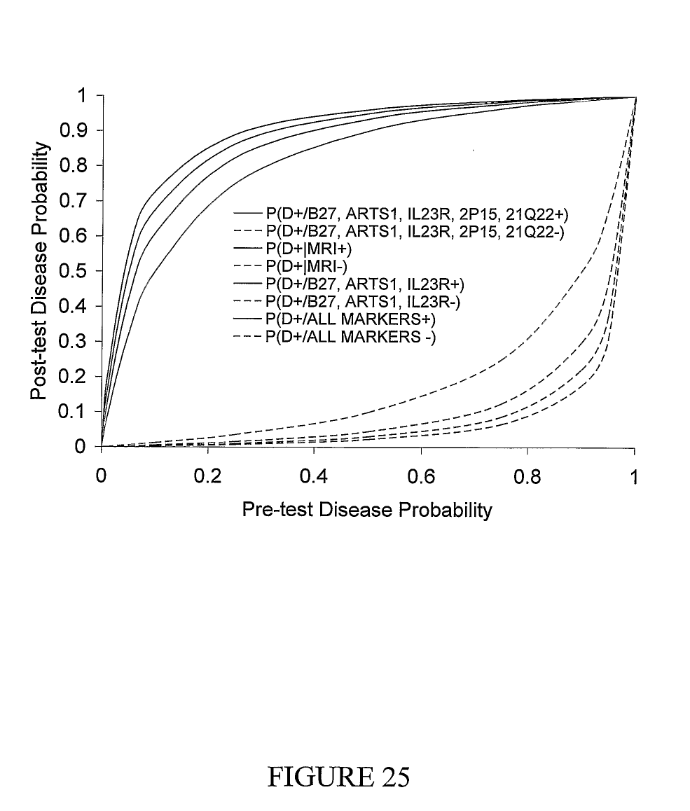

The diagnostic value of each of the AS markers were tested and the finding are reported in Table 5 below as the post-test probability of a diagnosis of AS calculated based on the pre-test probability of disease, and the genetic findings either of the individual marker, or combinations of markers, including the ARTS-1, IL-23R and B27 genes. The corresponding diagnostic value of MRI scanning, currently considered the most sensitive method for AS diagnosis is included for comparison. FIGS. 24 and 25 illustrate these findings in graphical format.

TABLE 5

| | Pre-test probability | | | | | | | | | | |
|---|---|---|---|---|---|---|---|---|---|---|---|
| | 0 | 0.004 | 0.01 | 0.05 | 0.1 | 0.25 | 0.5 | 0.75 | 0.9 | 0.95 | 0.9999 |
| B27 ALONE | | | | | | | | | | | |
| LR(G1) | | 11.13 | 11.13 | 11.13 | 11.13 | 11.13 | 11.13 | 11.13 | 11.13 | 11.13 | 11.13 |
| LR(G0) | | 0.11 | 0.11 | 0.11 | 0.11 | 0.11 | 0.11 | 0.11 | 0.11 | 0.11 | 0.11 |
| P(D+\|G1) | 0 | 0.04 | 0.10 | 0.37 | 0.55 | 0.79 | 0.92 | 0.97 | 0.99 | 1.00 | 1.00 |
| P(D−\|G0) | 1 | 1.00 | 1.00 | 0.99 | 0.99 | 0.97 | 0.90 | 0.76 | 0.51 | 0.33 | 0.00 |
| P(D−\|G1) | 1 | 0.96 | 0.90 | 0.63 | 0.45 | 0.21 | 0.08 | 0.03 | 0.01 | 0.00 | 0.00 |
| P(D+\|G0) | 0 | 0.00 | 0.00 | 0.01 | 0.01 | 0.03 | 0.10 | 0.24 | 0.49 | 0.67 | 1.00 |
| IL23R ALONE | | | | | | | | | | | |
| LR(G1) | | 1.06 | 1.06 | 1.06 | 1.06 | 1.06 | 1.06 | 1.06 | 1.06 | 1.06 | 1.06 |
| LR(G0) | | 0.57 | 0.57 | 0.57 | 0.57 | 0.57 | 0.57 | 0.57 | 0.57 | 0.57 | 0.57 |
| P(D+\|G1) | 0 | 0.00 | 0.01 | 0.05 | 0.11 | 0.26 | 0.51 | 0.76 | 0.91 | 0.95 | 1.00 |
| P(D−\|G0) | 1 | 1.00 | 0.99 | 0.97 | 0.94 | 0.84 | 0.64 | 0.37 | 0.16 | 0.08 | 0.00 |
| P(D−\|G1) | 1 | 1.00 | 0.99 | 0.95 | 0.89 | 0.74 | 0.49 | 0.24 | 0.09 | 0.05 | 0.00 |
| P(D+\|G0) | 0 | 0.00 | 0.01 | 0.03 | 0.06 | 0.16 | 0.36 | 0.63 | 0.84 | 0.92 | 1.00 |
| ARTS1 ALONE | | | | | | | | | | | |
| LR(G1) | | 1.19 | 1.19 | 1.19 | 1.19 | 1.19 | 1.19 | 1.19 | 1.19 | 1.19 | 1.19 |
| LR(G0) | | 0.76 | 0.76 | 0.76 | 0.76 | 0.76 | 0.76 | 0.76 | 0.76 | 0.76 | 0.76 |
| P(D+\|G1) | 0 | 0.00 | 0.01 | 0.06 | 0.12 | 0.28 | 0.54 | 0.78 | 0.91 | 0.96 | 1.00 |
| P(D−\|G0) | 1 | 1.00 | 0.99 | 0.96 | 0.92 | 0.80 | 0.57 | 0.30 | 0.13 | 0.06 | 0.00 |
| P(D−\|G1) | 1 | 1.00 | 0.99 | 0.94 | 0.88 | 0.72 | 0.46 | 0.22 | 0.09 | 0.04 | 0.00 |
| P(D+\|G0) | 0 | 0.00 | 0.01 | 0.04 | 0.08 | 0.20 | 0.43 | 0.70 | 0.87 | 0.94 | 1.00 |
| CHR2P15 | | | | | | | | | | | |
| LR(G1) | | 1.15 | 1.15 | 1.15 | 1.15 | 1.15 | 1.15 | 1.15 | 1.15 | 1.15 | 1.15 |
| LR(G0) | | 0.77 | 0.77 | 0.77 | 0.77 | 0.77 | 0.77 | 0.77 | 0.77 | 0.77 | 0.77 |
| P(D+\|G1) | 0 | 0.00 | 0.01 | 0.06 | 0.11 | 0.28 | 0.53 | 0.77 | 0.91 | 0.96 | 1.00 |
| P(D−\|G0) | 1 | 1.00 | 0.99 | 0.96 | 0.92 | 0.80 | 0.57 | 0.30 | 0.13 | 0.06 | 0.00 |
| P(D−\|G1) | 1 | 1.00 | 0.99 | 0.94 | 0.89 | 0.72 | 0.47 | 0.23 | 0.09 | 0.04 | 0.00 |
| P(D+\|G0) | 0 | 0.00 | 0.01 | 0.04 | 0.08 | 0.20 | 0.43 | 0.70 | 0.87 | 0.94 | 1.00 |
| CHR21Q22 | | | | | | | | | | | |
| LR(G1) | | 1.17 | 1.17 | 1.17 | 1.17 | 1.17 | 1.17 | 1.17 | 1.17 | 1.17 | 1.17 |
| LR(G0) | | 0.87 | 0.87 | 0.87 | 0.87 | 0.87 | 0.87 | 0.87 | 0.87 | 0.87 | 0.87 |
| P(D+\|G1) | 0 | 0.00 | 0.01 | 0.06 | 0.12 | 0.28 | 0.54 | 0.78 | 0.91 | 0.96 | 1.00 |
| P(D−\|G0) | 1 | 1.00 | 0.99 | 0.96 | 0.91 | 0.77 | 0.53 | 0.28 | 0.11 | 0.06 | 0.00 |
| P(D−\|G1) | 1 | 1.00 | 0.99 | 0.94 | 0.88 | 0.72 | 0.46 | 0.22 | 0.09 | 0.04 | 0.00 |
| p(D+\|G0) | 0 | 0.00 | 0.01 | 0.04 | 0.09 | 0.23 | 0.47 | 0.72 | 0.89 | 0.94 | 1.00 |
| TNFR1 | | | | | | | | | | | |
| LR(G1) | | 1.17 | 1.17 | 1.17 | 1.17 | 1.17 | 1.17 | 1.17 | 1.17 | 1.17 | 1.17 |
| LR(G0) | | 0.92 | 0.92 | 0.92 | 0.92 | 0.92 | 0.92 | 0.92 | 0.92 | 0.92 | 0.92 |
| P(D+\|G1) | 0 | 0.00 | 0.01 | 0.06 | 0.12 | 0.28 | 0.54 | 0.78 | 0.91 | 0.96 | 1.00 |
| P(D−\|G0) | 1 | 1.00 | 0.99 | 0.95 | 0.91 | 0.77 | 0.52 | 0.27 | 0.11 | 0.05 | 0.00 |
| P(D−\|G1) | 1 | 1.00 | 0.99 | 0.94 | 0.88 | 0.72 | 0.46 | 0.22 | 0.09 | 0.04 | 0.00 |
| P(D+\|G0) | 0 | 0.00 | 0.01 | 0.05 | 0.09 | 0.23 | 0.48 | 0.73 | 0.89 | 0.95 | 1.00 |
| TRADD | | | | | | | | | | | |
| LR(G1) | | 1.09 | 1.09 | 1.09 | 1.09 | 1.09 | 1.09 | 1.09 | 1.09 | 1.09 | 1.09 |
| LR(G0) | | 0.82 | 0.82 | 0.82 | 0.82 | 0.82 | 0.82 | 0.82 | 0.82 | 0.82 | 0.82 |
| P(D+\|G1) | 0 | 0.00 | 0.01 | 0.05 | 0.11 | 0.27 | 0.52 | 0.77 | 0.91 | 0.95 | 1.00 |
| P(D−\|G0) | 1 | 1.00 | 0.99 | 0.96 | 0.92 | 0.79 | 0.55 | 0.29 | 0.12 | 0.06 | 0.00 |
| P(D−\|G1) | 1 | 1.00 | 0.99 | 0.95 | 0.89 | 0.73 | 0.48 | 0.23 | 0.09 | 0.05 | 0.00 |
| P(D+\|G0) | 0 | 0.00 | 0.01 | 0.04 | 0.08 | 0.21 | 0.45 | 0.71 | 0.88 | 0.94 | 1.00 |
| ALL GWS COMBINED | | | | | | | | | | | |
| LR(G1) | | 18.83 | 18.83 | 18.83 | 18.83 | 18.83 | 18.83 | 18.83 | 18.83 | 18.83 | 18.83 |
| LR(G0) | | 0.03 | 0.03 | 0.03 | 0.03 | 0.03 | 0.03 | 0.03 | 0.03 | 0.03 | 0.03 |
| P(D+\|G1) | 0 | 0.07 | 0.16 | 0.50 | 0.68 | 0.86 | 0.95 | 0.98 | 0.99 | 1.00 | 1.00 |
| P(D−\|G0) | 1 | 1.00 | 1.00 | 1.00 | 1.00 | 0.99 | 0.97 | 0.91 | 0.78 | 0.63 | 0.00 |
| P(D−\|G1) | 1 | 0.93 | 0.84 | 0.50 | 0.32 | 0.14 | 0.05 | 0.02 | 0.01 | 0.00 | 0.00 |
| P(D+\|G0) | 0 | 0.00 | 0.00 | 0.00 | 0.00 | 0.01 | 0.03 | 0.09 | 0.22 | 0.37 | 1.00 |
| B27+ARTS1+IL23R | | | | | | | | | | | |
| LR(G1) | | 14.03 | 14.03 | 14.03 | 14.03 | 14.03 | 14.03 | 14.03 | 14.03 | 14.03 | 14.03 |
| LR(G0) | | 0.05 | 0.05 | 0.05 | 0.05 | 0.05 | 0.05 | 0.05 | 0.05 | 0.05 | 0.05 |
| P(D+\|G1) | 0 | 0.05 | 0.12 | 0.42 | 0.61 | 0.82 | 0.93 | 0.98 | 0.99 | 1.00 | 1.00 |
| P(D−\|G0) | 1 | 1.00 | 1.00 | 1.00 | 0.99 | 0.98 | 0.96 | 0.88 | 0.70 | 0.53 | 0.00 |
| P(D−\|G1) | 1 | 0.95 | 0.88 | 0.58 | 0.39 | 0.18 | 0.07 | 0.02 | 0.01 | 0.00 | 0.00 |
| P(D+\|G0) | 0 | 0.00 | 0.00 | 0.00 | 0.01 | 0.02 | 0.04 | 0.12 | 0.30 | 0.47 | 1.00 |
| ALL COMBINED | | | | | | | | | | | |
| LR(G1) | | 24.15 | 24.15 | 24.15 | 24.15 | 24.15 | 24.15 | 24.15 | 24.15 | 24.15 | 24.15 |
| LR(G0) | | 0.02 | 0.02 | 0.02 | 0.02 | 0.02 | 0.02 | 0.02 | 0.02 | 0.02 | 0.02 |
| P(D+\|G1) | 0 | 0.09 | 0.20 | 0.56 | 0.73 | 0.89 | 0.96 | 0.99 | 1.00 | 1.00 | 1.00 |
| P(D−\|G0) | 1 | 1.00 | 1.00 | 1.00 | 1.00 | 0.99 | 0.98 | 0.93 | 0.82 | 0.69 | 0.00 |
| P(D−\|G1) | 1 | 0.91 | 0.80 | 0.44 | 0.27 | 0.11 | 0.04 | 0.01 | 0.00 | 0.00 | 0.00 |
| P(D+\|G0) | 0 | 0.00 | 0.00 | 0.00 | 0.00 | 0.01 | 0.02 | 0.07 | 0.18 | 0.31 | 1.00 |
| MRI+ | | | | | | | | | | | |
| P(D+\|MRI+) | 0.00 | 0.03 | 0.08 | 0.32 | 0.50 | 0.75 | 0.90 | 0.96 | 0.99 | 0.99 | 1.00 |
| P(D+\|MRI−) | 0.00 | 0.00 | 0.00 | 0.01 | 0.01 | 0.04 | 0.10 | 0.25 | 0.50 | 0.68 | 1.00 |
| P(D−\|MRI−) | 1.00 | 1.00 | 1.00 | 0.99 | 0.99 | 0.96 | 0.90 | 0.75 | 0.50 | 0.32 | 0.00 |
| P(D−\|MRI+) | 1.00 | 0.97 | 0.92 | 0.68 | 0.50 | 0.25 | 0.10 | 0.04 | 0.01 | 0.01 | 0.00 |

### Example 3

### Detection of AS-Associated Polymorphisms within the IL-23R Sequence

### Patients

As part of the Wellcome Trust Case-Control Consortium, 1000 British Caucasian AS cases and 1500 healthy, ethnically matched controls drawn from the 1958 British Birth Cohort (BBC) were genotyped for 14,436 non-synonymous SNPs spread across the genome.

AS was defined according to the modified New York diagnostic criteria (Van der Linden, S et al., 1984, Arthritis Rheum, 27: 361-368). All patients had been seen by a qualified rheumatologist, and the diagnosis of AS confirmed. To confirm diagnosis all cases, patients were either examined or interviewed by telephone by one of the investigators. In cases with atypical histories or where radiographs had not been previously performed, pelvic and lumbo-sacral spine radiographs were obtained, and attending physicians contacted to confirm the diagnosis.

After examining the SNPs, the inventors noted a strong association between AS and a single genotyped SNP lying in IL-23R (rs11209026, P=0.001). Comparing the AS cases with these 3000 controls, association with this SNP was observed with (P=3×10⁻⁴).

To better define the association, eight IL-23R SNPs were genotyped in the same 1000 British AS cases and 1500 BCC controls, and in a further cohort of white North American AS cases (n=634) and healthy North American controls (n=672). The North American cases included Caucasian patients from two cohorts: 1) the prospective Study of Outcomes in Ankylosing Spondylitis (PSOAS), an observational study whose main aim was to investigate genetic markers of AS severity (n=390) and; 2) the North American Spondylitis Consortium, with 244 AS probands from families with two or more siblings both meeting modified 1984 New York criteria (van der Linden, S., et al., 1984, Arthritis Rheum, 27: 361-368).

### Genotyping of Polymorphisms within the IL-23R Sequence

Genotyping was performed with the iPLEX assay (MassArray, Sequenom) in the British samples, and by ABI TaqMan™ assay as described above in the North American samples.

Genotype and allele frequencies were similar between British and US cases and controls respectively (see Table 6, wherein minor allele frequencies (MAF) and odds ratios (OR) are illustrated). Association was tested in each dataset independently, and in the combined dataset with p-values determined by simulation with clustering within each dataset, using the program “PLINK” (http://pngu.mgh.harvard.edu/˜purcell/plink/).

### Statistical Analysis of IL-23R Polymorphisms

In the UK dataset, strong association was seen in seven of the eight genotyped SNPs (P≦0.002), with peak association seen at rs11209032 (P=6.8×10⁻⁶). In the North American dataset, association was observed with all genotyped SNPs (P≦0.03), with peak association observed with marker rs1343151 (P=3.8×10⁻⁵). In the combined dataset, the strongest association observed was with SNP rs11209032 (odds ratio 1.3, 95% CI1.2-1.4, P=3×10⁻⁸). The attributable risk fraction for this marker in the North American confirmation cohort was 12%.

### Example 4

### A Genotype Wide Scan of AS-Associated Polymorphisms within the IL-23R Sequence

The inventors completed one of the largest and most comprehensive scans conducted to date, involving the genome-wide association on 1000 individuals with AS and 1500 common control individuals using a dense panel of 14,436 markers. In addition to the scan of 1500 k markers, the inventors conducted a study of 5,500 independent individuals using a gene-based scan of coding variants.

### Sample Collection

In order to identify individuals who might have ancestries other than Western European, the inventors merged 60 CEU founder (US residents with northern and western European ancestry), 60 YR1 founder (from the Yoruba in Ibadan, Nigeria), 90 JPT founder (Japanese in Tokyo, Japan) and CHB founder (Hanchinese in Beijing China) individuals from the International HapMap Project (Altshuler, D et al., 2005, Nature, 437: 1299-1320). Individual AS cases or healthy controls with genotype patterns similar to groups other than CEU were removed from the analysis. Any individual with >10% of genotypes missing was also removed from the analysis.

### Genotyping

Initial genotyping involved 14,436 SNPs. At the time of study inception, this comprised the complete set of known SNPs with minor allele frequencies (MAF)>1% in Caucasian samples. In addition, the inventors also typed a dense set of 897 SNPs throughout the major histocompatibility complex (MHC), as well as 103 SNPs in pigmentation genes specifically designed to differentiate between population groups.

SNP genotyping was performed with the Infinium I assay (Illumina) which is based on Allele Specific Primer Extension (ASPE) and the use of a single fluorochrome. The assay requires ˜250 ng of genomic DNA which is first subjected to a round of isothermal amplification generating a “high complexity” representation of the genome with most loci represented at usable amounts. There are two allele specific probes (50mers) per SNP each on a different bead type; each bead type is present on the array 30 times on average (minimum 5), allowing for multiple independent measurements. The inventors processed six samples per array. Clustering was performed with the GenCall software version 6.2.0.4 which assigns a quality score to each locus and an individual genotype confidence score (GC score) which is based on the distance of a genotype from the centre of the nearest cluster. Primarily, the inventors removed samples with more than 50% of loci having a score below 0.7 and then all loci with a quality score below 0.2. Post clustering we applied two additional filtering criteria: (i) omit individual genotypes with a GC score <0.15 and (ii) remove any SNP which had more than 20% of its samples with GC scores below 0.15. The above criteria were designed so as to optimize genotype accuracy whilst minimizing uncalled genotypes.

One of the strongest associations observed in the study was between MHC and AS with p-values of <10⁻²⁰. The extent of MHC association observed in AS was broad. For example, in AS, association was observed at p<10⁻⁵⁰ across >1.5 MB. The inventors hypothesised that this may be due either to extreme linkage disequilibrium with HLA-B27, or the presence of more than one MHC susceptibility gene operating in these diseases.

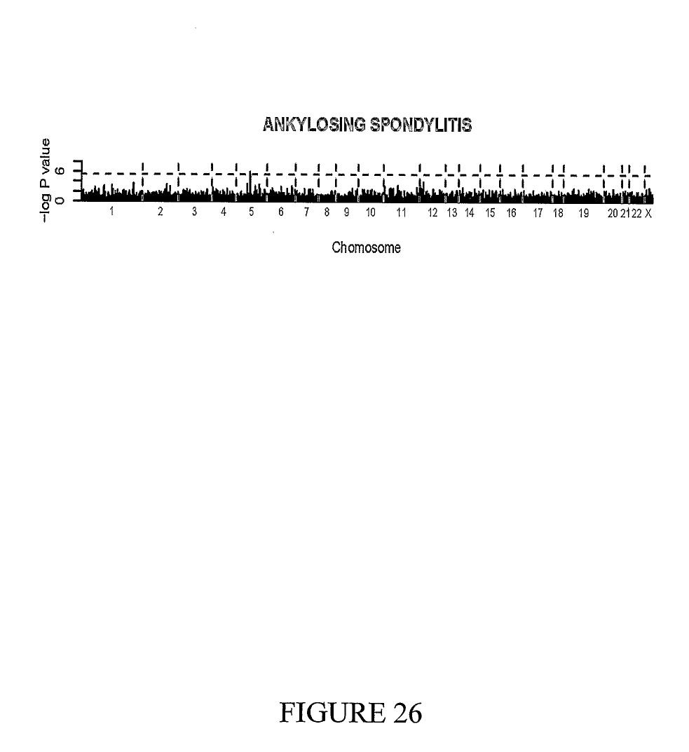

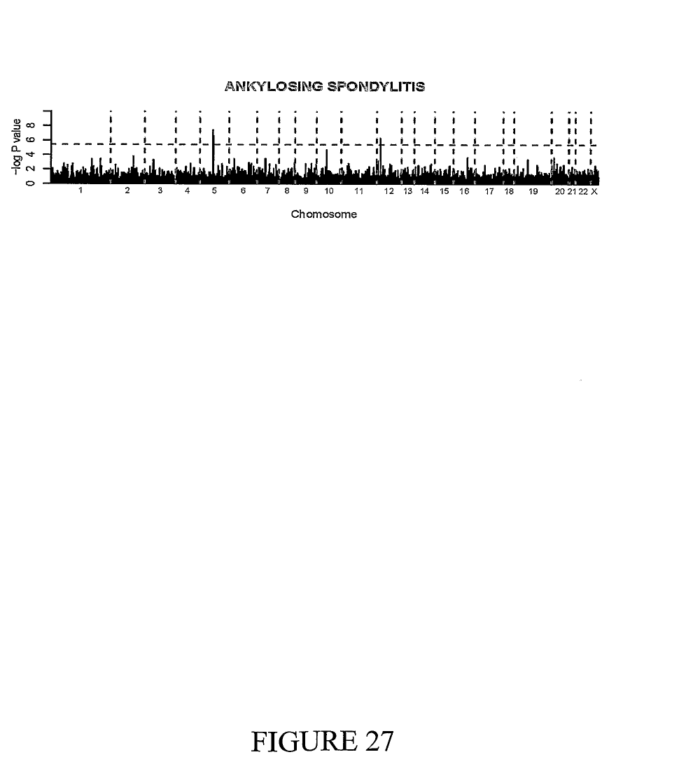

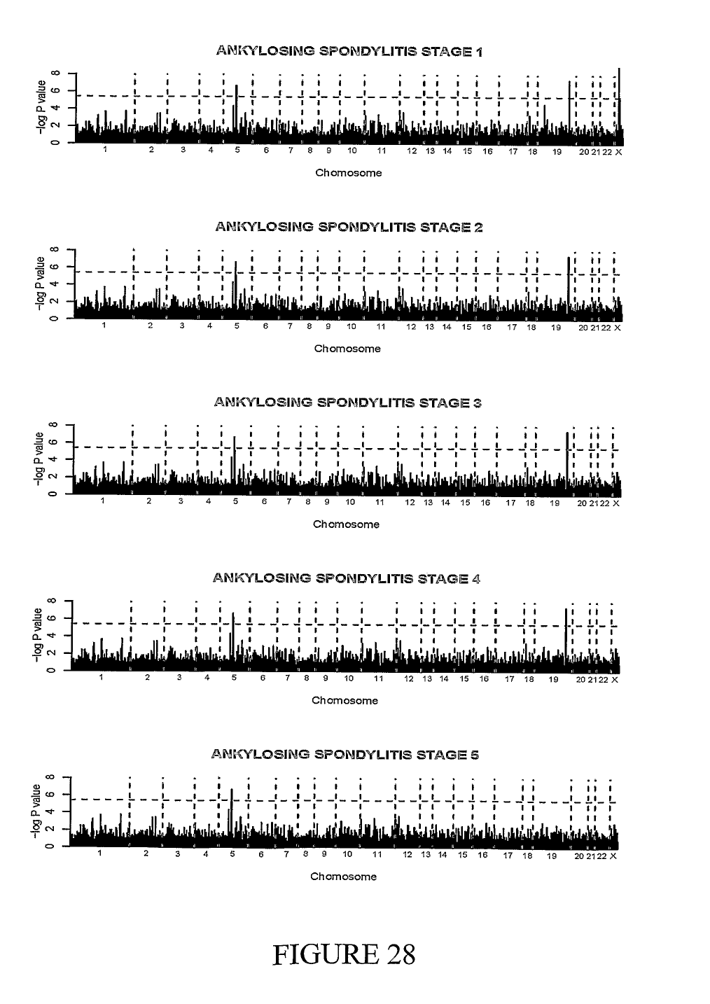

FIG. 26 displays the results for the Cochrane-Armitage trend-test for AS following data clean-up. FIG. 27 displays the results for the Cochrane-Armitage trend-test for AS with combined controls following data clean-up and FIG. 28 displays the results for the Cochrane-Armitage significance tests after each stage of genotype filtering for Ankylosing Spondylitis. In addition, two SNPs on chromosome 5 reached permutation-based and Bonferroni genome-wide significance at p<0.05 for Ankylosing Spondylitis (rs27044: χ²=23.90, p=1.0×10⁻⁶; rs30187: χ²=21.82, p=3.0×10⁻⁶).

### Statistical Analysis

Markers that were monomorphic in both case and control samples, SNPs with >10% missing genotypes, and SNPs with differences in the amount of missing data between cases and controls (p<10⁻⁴ as assessed by χ² test) were excluded from all analyses involving that case group only. In addition, any marker which failed an exact test of Hardy-Weinberg equilibrium in controls (p<10⁻⁷) was excluded from all analyses (Wigginton, J. E et al., 2005, Am J Hum Genet, 76: 887-893)

Cochrane-Armitage Tests for trend (Armitage, P, 1955, Biometrics, 11: 375-386) were conducted using Purcell's PLINK program (http://pngu.mgh.harvard.edu/˜purcell/plink). The inventors' evaluated statistical significance against a Bonferroni corrected threshold, as well as performing 1000 case-control permutations of the data to provide genome-wide significance values. Any marker with an asymptotic significance value of p<10⁻³ on the trend test had its raw intensity values rechecked for possible problems in the calling algorithm.

Whilst great lengths were taken to ensure the samples were as homogenous as possible in terms of genetic ancestry, even subtle population substructure can substantially influence tests of association in large genome-wide analyses involving thousands of individuals (Marchini, J et al., 2004, Nat Genet, 36: 512-517). The inventors therefore calculated the genomic-control inflation factor, λ (Devlin, B and Roeder, K, 1999, Biometrics, 55: 997-1004) for each case-control sample as well as in the analyses where the inventors combined the other case groups with the control individuals. In general, values for λ were small (˜1.1) indicating a small degree of substructure in UK samples and necessitating only a slight correction to the test statistic (WTCCC, Nature Genetics (in review).

Power calculations were performed using the Genetic Power Calculator (http://pngu.mgh.harvard.edu/˜purcell/gpc). LD coverage estimates and allele frequencies were based on pre-computed scores from the International HapMap website.

TABLE 6
ASSOCIATION STUDY FINDINGS FOR IL-23R

| | | | | | | | | | | | | | CASES WITH NO | | |
|---|---|---|---|---|---|---|---|---|---|---|---|---|---|---|---|
| | UK CASES | | | | US CASES | | | | ALL CASES | | | | CLINICAL IBD | | |
| | Case | Control | | | Case | Control | | | Case | Control | | | Case | | |
| SNP | MAF | MAF | OR | P-value | MAF | MAF | OR | P-value | MAF | MAF | OR | P-value | MAF | OR | P-value |
| rs1004819 | 0.35 | 0.3 | 1.2 | 0.001 | 0.35 | 0.31 | 1.2 | 0.01 | 0.35 | 0.30 | 1.2 | 1.1 × 10⁻⁵ | 0.36 | 1.3 | 3.8 × 10⁻⁵ |
| rs10489629 | 0.43 | 0.45 | 0.9 | 0.072 | 0.39 | 0.47 | 0.73 | 0.00014 | 0.41 | 0.46 | 0.83 | 0.00011 | 0.4 | 0.8 | 5.1 × 10⁻⁵ |
| rs11465804 | 0.043 | 0.061 | 0.68 | 0.0043 | 0.043 | 0.063 | 0.67 | 0.03 | 0.043 | 0.061 | 0.68 | 0.00041 | 0.044 | 0.7 | 0.0059 |
| rs11209026 | 0.042 | 0.064 | 0.64 | 0.0008 | 0.039 | 0.064 | 0.6 | 0.006 | 0.041 | 0.063 | 0.63 | 2.8 × 10⁻⁵ | 0.042 | 0.65 | 0.00082 |
| rs1343151 | 0.3 | 0.34 | 0.85 | 0.0089 | 0.29 | 0.37 | 0.7 | 3.8 × 10⁻⁵ | 0.30 | 0.34 | 0.8 | 1.0 × 10⁻⁵ | 0.29 | 0.78 | 3.5 × 10⁻⁵ |
| rs10889677 | 0.36 | 0.31 | 1.2 | 0.0014 | 0.37 | 0.30 | 1.4 | 0.00013 | 0.36 | 0.31 | 1.3 | 6.3 × 10⁻⁷ | 0.37 | 1.3 | 1.2 × 10⁻⁶ |
| rs11209032 | 0.38 | 0.32 | 1.3 | 6.8 × 10⁻⁶ | 0.38 | 0.32 | 1.3 | 0.00097 | 0.38 | 0.32 | 1.3 | 3.5 × 10⁻⁸ | 0.38 | 1.3 | 6.9 × 10⁻⁷ |
| rs1495965 | 0.49 | 0.44 | 1.2 | 0.0023 | 0.51 | 0.43 | 1.3 | 0.00024 | 0.49 | 0.44 | 1.2 | 3.1 × 10⁻⁶ | 0.5 | 1.3 | 4.1 × 10⁻⁶ |

### Example 5

### Detection of AS-Associated Polymorphisms within the ARTS-1 Sequence

### Patients

As part of the Wellcome Trust Case-Control Consortium, 1000 British Caucasian AS cases and 1500 healthy, ethnically matched controls drawn from the 1958 British Birth Cohort (BBC) were genotyped for 14,436 non-synonymous SNPs spread across the genome.

AS was defined according to the modified New York diagnostic criteria (Van der Linden, S et al., 1984, Arthritis Rheum, 27: 361-368). All patients had been seen by a qualified rheumatologist, and the diagnosis of AS confirmed. To confirm diagnosis all cases, patients were either examined or interviewed by telephone by one of the investigators. In cases with atypical histories or where radiographs had not been previously performed, pelvic and lumbo-sacral spine radiographs were obtained, and attending physicians contacted to confirm the diagnosis.

To better define the association, five ARTS-1 SNPs were genotyped in the same 1000 British AS cases and 1500 BCC controls, and in a further cohort of white North American AS cases (n=634) and healthy North American controls (n=672). The North American cases included Caucasian patients from two cohorts: 1) the prospective Study of Outcomes in Ankylosing Spondylitis (PSOAS), an observational study whose main aim was to investigate genetic markers of AS severity (n=390) and; 2) the North American Spondylitis Consortium, with 244 AS probands from families with two or more siblings both meeting modified 1984 New York criteria (van der Linden, S., et al., 1984, Arthritis Rheum, 27: 361-368).

### Genotyping of Polymorphisms within the ARTS-1 Sequence

Genotyping was performed with the iPLEX assay (MassArray, Sequenom) in the British samples, and by ABI TaqMan™ assay as described above in the North American samples.

Genotype and allele frequencies were similar between British and US cases and controls respectively (see Table 7, wherein minor allele frequencies (MAF) and odds ratios (OR) are illustrated). Association was tested in each dataset independently, and in the combined dataset with p-values determined by simulation with clustering within each dataset, using the program “PLINK” (http://pngu.mgh.harvard.edu/˜purcell/plink/).

TABLE 7
ASSOCIATION STUDY FINDINGS FOR ARTS-1

| | UK CASES | | | | US CASES | | | | ALL CASES | | | |
|---|---|---|---|---|---|---|---|---|---|---|---|---|
| | Case | Control | | | Case | Control | | | Case | Control | | |
| SNP | MAF | MAF | OR | P-value | MAF | MAF | OR | P-value | MAF | MAF | OR | P-value |
| rs27044 | 0.34 | 0.27 | 1.4 | 1.6 × 10⁻⁷ | — | — | — | — | — | — | — | — |
| rs17482078 | 0.17 | 0.22 | 0.75 | 0.00013 | 0.15 | 0.21 | 0.65 | 5.1 × 10⁻⁵ | 0.16 | 0.22 | 0.7 | 1.2 × 10⁻⁸ |
| rs10050860 | 0.18 | 0.23 | 0.74 | 7.7 × 10⁻⁵ | 0.15 | 0.22 | 0.66 | 8.8 × 10⁻⁵ | 0.17 | 0.22 | 0.71 | 7.6 × 10⁻⁹ |
| rs30187 | 0.41 | 0.33 | 1.4 | 4.4 × 10⁻⁷ | 0.41 | 0.35 | 1.3 | 0.00047 | 0.41 | 0.34 | 1.4 | 3.4 × 10⁻¹⁰ |
| rs2287987 | 0.18 | 0.22 | 0.75 | 0.00011 | 0.15 | 0.21 | 0.66 | 8.4 × 10⁻⁵ | 0.17 | 0.22 | 0.71 | 1.0 × 10⁻⁸ |

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

1-62. (canceled)

63. A method of diagnosing the presence or risk of development of Ankylosing Spondylitis (AS) in a subject, comprising analyzing a biological sample obtained from the subject for the presence of a polymorphism in an AS marker selected from the group consisting of an ARTS-1 gene and an expression product of an ARTS-1 gene, wherein the polymorphism is selected from the group consisting of: a G (guanine) at reference sequence (rs) 27044; a T (thymine) at rs30187; and a C (cytosine) at rs17482078, wherein the presence of the polymorphism indicates that the subject has AS or is at risk of developing AS.

64. A method according to claim 63, wherein the sample is further analyzed for the presence of at least one other polymorphism in the AS marker, which is indicative of the presence or risk of development of AS, wherein the at least one other polymorphism is selected from the group consisting of: T at rs2287987; and C at rs10050860.

65. A method according to claim 63 or claim 64, wherein the sample is further analyzed for the presence of a further polymorphism in at least one other AS marker, wherein the further polymorphism is indicative of the presence or risk of development of AS, and wherein the other AS marker is selected from the group consisting of an IL-23R gene; a TNFR1 gene locus; a TRADD gene locus; a 21Q22 chromosome locus, an IL-1R1 gene locus, an IL-1R2 gene locus, a CD74 gene locus, a 2Q31.3 chromosome locus and a 4Q13.1 chromosome locus.

66. A method according to claim 65, wherein the further polymorphism is a polymorphism in the IL-23R gene selected from the group consisting of a T at rs11465804 or rs10489629; a G at rs11209026 or rs1343151; a C at rs1495965; and an A (adenine) at rs1004819, rs10889677 or rsl 1209032;

67. A method according to claim 65, wherein the further polymorphism is a polymorphism in the TNFR1 gene locus, represented by a C at rs4149576.

68. A method according to claim 65, wherein the further polymorphism is a polymorphism in the TRADD gene locus, represented by a G at rs9033.

69. A method according to claim 65, wherein the further polymorphism is a polymorphism in the 2P15 chromosome locus, represented by an A at rs10865331.

70. A method according to claim 65, wherein the further polymorphism is a polymorphism in the 21Q22 chromosome locus, represented by a G at rs2242944.

71. A method according to claim 65, wherein the further polymorphism is a polymorphism in the IL-1R1 gene locus represented by a C at rs949963.

72. A method according to claim 65, wherein the further polymorphism is a polymorphism in the IL-1R2 gene locus represented by a T at rs2310173.

73. A method according to claim 65, wherein the further polymorphism is a polymorphism in the TCOF1 gene, which is in genetic linkage with the CD74 gene locus, represented by a C at rs 15251.

74. A method according to claim 65, wherein the further polymorphism is a polymorphism in the chromosomal locus 2Q31.3 represented by a C at rs1018326.

75. A method according to claim 65, wherein the further polymorphism is a polymorphism in the chromosomal locus 4Q13.1 represented by a G at rs10517820.

76. A method according to any one of claim 63, wherein the subject is selected from the group consisting of: an adult, child, fetus and embryo.

77. A method according to claim 63, wherein the sample from the subject is obtained from a tissue or fluid selected from the group consisting of: hair, skin, nails, saliva and blood.

78. A method according to claim 63, wherein the subject is caucasian.

79. A method for treating AS in a subject, comprising: (a) analyzing a biological sample obtained from the subject for the presence of a polymorphism in an AS marker selected from the group consisting of an ARTS-1 gene and an expression product of an ARTS-1 gene, wherein the polymorphism is selected from the group consisting of: a G (guanine) at reference sequence (rs) 27044; a T (thymine) at rs30187; and a C (cytosine) at rs17482078, and (b) exposing the subject to a treatment that ameliorates or reverses the symptoms of AS on the basis that the subject tests positive for the polymorphism(s).


SEQUENCE LISTING (22 sequences)

SEQ ID NO: 1 - DNA, 701 bp, Homo sapiens
  variation (201)..(201): C or T (ambiguity code Y)
ggtgtaggtatgttaacatgcttgataatggaagagtgaggatacctgcttcttaaacagggcctgctcatttttttctggcctctagtacatttctttctggcaaaactcaagctctgttttcacggagatgatgcactgagccatggcatccctaggtggtttgattcatttctgcaccccggtgtctagcaaagtgcyacgtgcatgtgtttaatcaaaatagcttgtggttgtcaagctctttgtattcttccctccctccctgtgatttgatttcctgaaagtatcctttccccaaggattatggaacatacacttgttaaacactggacccattttcatgatcatttaaatgtcatgacctttctgacaccttgatatggtttggctctgtgtccccacccaaatctcatctaaattataatccccatgtgtccagggagggatctggtgggaggtgattagatcatgggggcagtttttcctatgctgttctcatgatagtgagtgaattctcatgagatctgatggtttaaaagtgtttggcacttccccccttgctctctctcctgctgtcatgtaagacgtgcctacttccacttccaccatgattgtaagtttcctgaggcttccccagccatgcggagctgtaagtcaattaaatcctctttcctttataaattacccagtctcagata

SEQ ID NO: 2 - DNA, 951 bp, Homo sapiens
  variation (500)..(500): T or G (ambiguity code K)
ggaaaactgtaatctcaaactacttccagagaagtacaactatcattcctgtctactctccactactttgtcagagcttttaaacattttactagcttgcaattttactgcgtggaagacttgcaagtcatctgcaagggtgaggtttccctgtgtgctcactcattttaggtcatggagaagtattcatttgtcctgatggttggttttggtctccgggtttcctgtctagacccgctaaatgcctactggtctcatggctgcattttgaagtcatcaggttagacttggggcatgtttgaagtaggatctgaatccaggggaggcaatggcttcctgacaagggtagtttctcaaaacctgggattttcactcctgcaagttaggagtgggcatgatgagaatataaaatcatgaagcaactcaaatcagacttacttagtatactagaacaaggagggtatcccttgatgttggcagggacaaagaaagtgaatttkctgtttaaatatcattaaggaagtgtgtggaagtcagtcacgaaggggtggtgcagactaaaacaccaaaaggttcaaatgagctctgagtcaatcaatgcgtgattaataagtaaggaatcacttcagggaattaggggtgtttcttttattaaaaaaaattaaaaatagaactatcatacaatccagcaatcttactactgggtatatatccaaaggaaataaaatgagtatgcggaagagatatctgcacccccccaccccgtttattgccgcactattcataatagccaagatgtagaacaaacctaagtgtccaacagtgaatgaatgggtgaagaagatgtggtgcatatacacaatggaatactattcagctatataaaagaaggaaatgttgttctttgtgacaacatgaatgaatctggaggacgctatgctaaatgaaata

SEQ ID NO: 3 - DNA, 801 bp, Homo sapiens
  variation (401)..(401): C or T (ambiguity code Y)
tctctctccataggtggaaaagaggctgcttcaggcaccacacctcagaagtcccggaagcccaagaaaggggctgggaacccccaagcctcaaccctggcgctgcaaagcaacatcacccagtgcctcctgggccaaccctggcccctgaatgaggcccaggtgcaggcctcagtggtgaaggtcctgactgagctgctggaacaggaaagaaagaaggtggtggacaccaccaaggagagcagcaggaagggctgggagagccgcaagcggaagctatcgggagaccagccagctgccaggacccccaggagcaagaagaagaagaagctgggggccggggaaggtggggaggcctctgtttccccagaaaagacctccacgacttccaaggggaaagyaaagagagacaaagcaagtggtgatgtcaaggagaagaaagggaaggggtctcttggctcccaaggggccaaggacgagccagaagaggagcttcagaaggggatggggacggttgaaggtggagatcaaagcaacccaaagagcaagaaggagaagaagaaatccgacaagagtgagtgaccgcttctcccagcccaccccaagggctgctgggcaccccacgggggcgggagggaccctcagccagcacctggtctcattcctcccatgtagaaaagagaggctgagaccagcctggccaacatggcaaaaccccgtctctactaaaaatacaaaaaattagctggatgtggtggcgggcacctgtaatctcagctacatgggaggctgaggcaggag

SEQ ID NO: 4 - DNA, 601 bp, Homo sapiens
  variation (301)..(301): T or C (ambiguity code Y)
tggtcacagaggtaattggtggatttgaggattcacaggattctaaaatcgagttgctgcactgccttctttaaaaaattaacatttttgagttttgtctaggaagtgggagtaggaaatgaacaatttgttttgtcataagtagaagtatgagaggaacacttatctttaagaataagtgcagactttttgctctcctcaaaatatcatcttcattataattccaaaaatataatgaaagaaaaattaaaatgataacctatcttattcttacaattaacttaagatttttttgagttayttccatctatatactggaatcttaatactgaccttggcattaaagtccaggtagtattaataatatgaatgttacatttgtgttattattctacagtttattaagtgcttttacttatatagtggatttaattcttctgaatacttcataattatatccattttaataatatgaaggtgagtgttagagctctcaagcatacagtagacggctaagttagggctagcctatagattctctgactttggtccagcacgaaggcttttctatgacaaagccctttaaatgcatgaaacttat

SEQ ID NO: 5 - DNA, 201 bp, Homo sapiens
  variation (101)..(101): A or G (ambiguity code R)
gttgttgtgtttacagtaatatattcatttctgacatttcttatctgaaaaatctccctgttttaaaataatattttcattatcaactgttactaattacrtttgatctgggactcattctcatacacaggtcaaatatgtacacatattactagcaactagtattgatcacgttttgctgatattttaagaaaacttctg

SEQ ID NO: 6 - DNA, 601 bp, Homo sapiens
  variation (301)..(301): S = G/C
acttttatgcatagaagttccctcctagcatttcttaaagttacgagatacatagataaatcttaaaaagcaatttattttccagaaaagggcagcaggggagacacttaaactttttgttttccctaatgtttagtaccaatgatggaaaacagaaaagatgcccttcacatcaacaaattggttatttagtaaggactgacctcaagtttccattggattccttccactttctgaaatagccttctgccctctgtacgcacggctgatagttgtgcacacaggcgaggagtagtagttsactccgcagcattcgctctgagactgagccctcgtctgtccatgtctgcttatcaatgaggtcccttagcagcctgatgaggaaggcctgagggcgttgtacagggaaacagggaccagtattgtcacaggtcatttcatgttaatataatccattatggtctcagatgatgggggctaatttttaaagcataatctaacttttactgtataaatcatgcagcactgtttaattataaaagggccaaaaacaataaataatgatgaatgactttcatattttcataacaaacttttataa

SEQ ID NO: 7 - DNA, 601 bp, Homo sapiens
  variation (301)..(301): Y = C/T
aagttccctcctagcatttcttaaagttacgagatacatagataaatcttaaaaagcaatttattttccagaaaagggcagcaggggagacacttaaactttttgttttccctaatgtttagtaccaatgatggaaaacagaaaagatgcccttcacatcaacaaattggttatttagtaaggactgacctcaagtttccattggattccttccactttctgaaatagccttctgccctctgtacgcacggctgatagttgtgcacacaggcgaggagtagtagttcactccgcagcattygctctgagactgagccctcgtctgtccatgtctgcttatcaatgaggtcccttagcagcctgatgaggaaggcctgagggcgttgtacagggaaacagggaccagtattgtcacaggtcatttcatgttaatataatccattatggtctcagatgatgggggctaatttttaaagcataatctaacttttactgtataaatcatgcagcactgtttaattataaaagggccaaaaacaataaataatgatgaatgactttcatattttcataacaaacttttataagatatcctatctac

SEQ ID NO: 8 - DNA, 601 bp, Homo sapiens
  variation (301)..(301): Y = T/C
tactgagaataaccaaggtttaaagcctccttaatcctactgggaagatgggggcagggagtataagtcaacttcataggatacacagatgcagctgaaatattttagcaatagtggtaagggcattatttcaaaagcaaggttccatcctacaaggcagatgttacctagacaacaaaacaaattttactctaggagcattacccagtgtccggggcgccgtcagagcccttcatgtagtgctcttgcttcatgtgtacattcctccccctcactgtgatggttattaggggaaaacccytctgcagtgtccaagtgttcatcatggttttcacatccaccccttcctgatgccaatgctggtgtgtacacaaaaaggcagacacatcacccatttaacccaactctaagaaaagacagaacaccagcttatgctcaaacagccgctaccagttgccaacctcccctcttaggagcactttctgatccctttctcagtgtgatgtccccagccatcacacctccattagagcaccagctgtttgcttgtcattttcctccactacacatacgtgtctggaggacaggtagcatacctagt

SEQ ID NO: 9 - DNA, 201 bp, Homo sapiens
  variation (101)..(101): Y = T/C
actctataactgcctagcaagattatgcaaattgataactaccatttatcatttacgaagtactcctgtgtataagcttgtttgattatgatgtcagccayatttggtagtgtaattagcgctactttacaaaagcggaaactgggcatgacttactaaatagtacattgctggtgggtaatgacacctaaactataacaa

SEQ ID NO: 10 - DNA, 601 bp, Homo sapiens
  variation (301)..(301): R = G/A
ggaagtgtgacaatttttaaatttgtcaattccttaccaattgttttagtgttttagcattcagccccaaagtcccttactcttggcccccttcagttttttccctcctgtgatggtattagtaaagatctatggttagttatttaaaatgagactttgagaaaagcaagacccttggatttctaaattttactgatgcattgagtatttctaagctgctcgatagattagagttgtttggtgtggcagttccccagtgtgtccagttgctcacaaattttgacttgaatgttctttgccraattggcactgagtttctccttcttgccatcatttgcttcatgaaataatctttctttcgtttacatttataatcaagtgcagtagaaagattttaaatgagctattataaagtctactaatgatttcttatctacataggttttttgctcagaacttaatatttcaaaatttaaattacacattaataaacatattcctaatacccttgtaaggaaggcaaactaatacggaattttatttgaggctgttttaaaatatacttgattatgaagtcccttgaaatattttaatgttcaaa

SEQ ID NO: 11 - DNA, 601 bp, Homo sapiens
  variation (301)..(301): M = C/A
ggtcaaaatcaatatgagaaagctgccttgcaatctgaacttgggttttccctgcaatagaaattgaattctgcctctttttgaaaaaaatgtattcacatacaaatcttcacatggacacatgttttcatttcccttggataaatacctaggtaggggattgctgggccatatgataagcatatgtttcagttctaccaatcttgtttccagagtagtgacatttctgtgctcctaccatcaccatgtaagaattcccgggagctccatgcctttttaattttagccattcttctgcctmatttcttaaaattagagaattaaggtcccgaaggtggaacatgcttcatggtcacacatacaggcacaaaaacagcattatgtggacgcctcatgtattttttatagagtcaactatttcctctttattttccctcattgaaagatgcaaaacagctctctattgtgtacagaaagggtaaataatgcaaaatacctggtagtaaaataaatgctgaaaattttcctttaaaatagaatcattaggccaggcgtggtggctcatgcttgtaatcccagcactttggtaggctgaggtagg

SEQ ID NO: 12 - DNA, 601 bp, Homo sapiens
  variation (301)..(301): R = G/A
cttctactattttcaattgagagccctagctaatacaactctccaaaattaaaaaaagaaaaaagaaaaagaaaagaaaaaaagctttgtatttttaggctcttagaactcacattattttcttttaataatgattagcaacaactaatggtgtttgttttatcttgtacgctaaattatctgaaattgggtaggtactacagtggaaataaatatttgatgttattttcaataaattgttactggagttaaacctcttgctatcctgacaattcctccctacatcaccctctttgcaatrgcagatggaagaattggcaataaatgcaattcagcttgaagaaaacaccctaaatattagaaacctgtgaagaaccaccggattgccttatcaactcattgtgcatcctatttggaagtctaaaatagaactaaaattctaatacttttacttgtataaatgcttataattgtcatctttgcatctcagcaattattcctggataaatatactgcaaatcatctaagaagagaaaaacccctctgaattacatgactgagtttcagaatgtgagtaaagtatggctaaccaaaatgttca

SEQ ID NO: 13 - DNA, 601 bp, Homo sapiens
  variation (301)..(301): Y = C/T
accacatcacaatgcatttgtggatattttcctcgggattgctctgaaatcatttttcactgtaagccatcttggttactaggctgtaagctctttgagagcagcaaaatcctgtttgctttgccacagtctctcctgtatcattcttccctactcccctaccccacttctctctctctctctctctctctctcacacacacacacacacacacacacacacacacactgtgagctctgcattgcccactgaatggtgagaaattcagaaggatgttgcattccctttgttttccctttcytccagctccaggccaggcttctgtcccttaggagacataatcccacctgggctgaagcaccccaggacgcaggcacttccctttacctgttcaggctacctttaccctctgttttcccaacacaggtagccacccagtaattactactagtcacagcaatataaaaactttattgagtgctttctatttgctacaacctattcccctgatggactgtcaaagctgtgggcagggattttgttctctgctgtaatcccagcacctatcacagtgtctagcccagagcgcacacttaataaa

SEQ ID NO: 14 - DNA, 600 bp, Homo sapiens
  variation (301)..(301): R = A/G
gccccatcctggctggcagggctgcatgtttccatcctgtttgcctcttttatttaatgccaaagttttagccaaagacatcttcctacttttgttgtgtttcagcattctgttctgcactgtgggctggctctctgccccaaccctgggcactggcccctggctgggccatttcatggctcaaagcctctgggggctcaaggaaggctgggctgctcagtcccttcatgggtcttgctaatggaaagtagcatatatgtgctttaaaaatattaatccttttgaaaagaactgagaagaraaatgtataattttatcccatttttaatattttggtctagcaacttgtgatacatagatgacaattttgtgagtttttcaaatgtgtgtacagatttttgtaaatatgactcttttgtaattaactcatgtacagcctcatcctgtatagtttaatgatgaatgtgcaggggacctgtctcaggctcctatatggttcctgggccttatagccaggtttgtgtggcgctcccgacttttgtgactgactggtgtcttcccatttggactgtggcctggccagagccccttgcatatccc

SEQ ID NO: 15 - DNA, 1001 bp, Homo sapiens
  variation (501)..(501): R = G/A
gatcccatctctaaaaaaataaaatataaaatgtttaaaaaaaaaaaaaaaaaagcaaaaaagaccagcacggtggcttacgcctgtaatcccactgtaatcccagcactttgggaggccgaggcgggtggatcatgaggtcgggagttcaagaccagcctgaccaacatgatgaaaccccgtctccactaaaatacaaaaattagccaggcgtggtgatgggtgcctgtaatcccagctagtcaggaggctgaggcaggagaatcgcttgaacctgggagactgagtcaggagaaccgcttgaacccaggaggtggaggttggagtgagctgagattgcgccactgcactccagcctggttgacagcgcaagactcccttccccccccaaaaaaatctcctaatcttctgtgtctgtgtggcaagccatgaactccttctcagaagaatgtttttaaatgcattaaataaagcatgaggtggggagagttccaaaggaarccagtgatactaaaacatacagaattatcaaacagtgtttatgcttctgttataagaatagatgggctactttcacatattaaacagcaagagctagcagcaagtctagtgaactgggcacttggaagcagtgatgagcatagcagtatctctggatagctgtaacactgtgtgcaggtatctggggtttctgtcgtgaccacgtggcaggagctgctgccactgctgtctgatgctcgccccacagtggaaggagatgctaaattccgttacgcattagaggtcagtgaaaaggaagatgcagtttgttcccgtccaggcacaaggactcttgaatttgtccatagttaagaacggctcatccaggagcagagcgagaggccgggctgcgcgtcctcatctcctctcccagccttcgcatcctcctggctgcctcgcgtttcctccacgggcctggctgaacgcacacacaggcctgggggagactgcagagacacatc

SEQ ID NO: 16 - DNA, 601 bp, Homo sapiens
  variation (301)..(301): Y = C/T
tctggccatacatatgatataacccagtaatgttaacaaagtaatagtgaatgttcactggtaatccctcctttttagcattatgtaattcagctataatctctatctggcaagatattggccattcttctttgaaaccagaaagcattttcaatctgatcccttttgctttaaccaagataaatagcaaaagaaacagcactttaatcctacacattttcacattcctccttgaattaactagtagtttccaaaataaattacctgtttttgtttttagcaaaaatcgatggaccatgtyggatttgctggtgatgaatgtcaatggaacatgccacaggtacctaaaagaaaggagaggtggattgttcctttgttgattcaattcaacaagcagttattaaatcacctatcatgtgccaggcactggttagtttagaacaagatagtaccaaaactgctacagccaagctctcaaaacatccccagtctgttttccagctttgtagggagtgtcagctcgggcactatttccttttcttggactccttgagtagagctaagaatttggactcacgtgaaccctgaaagcctttttaca

SEQ ID NO: 17 - DNA, 601 bp, Homo sapiens
  variation (301)..(301): Y = T/C
ccataaacaaagacactcagaaagaagtatattttagatgtccaatataagtttatgaatgttaacatatatttacaggaccaatatttaatcaaggtaaaaataaaaaaagttcaacatgcattataagacataaaaacgaatgctttggagaaacaatttttcctattaaaaaaagacaaagtaaaaattatataaatagccttatatgtcccatagaagcaatataaatgtagtttatactttattaaaagtgtgaatgagcttatacctggtgggccagttcatgggccacagtcaytgtgatgccaagcttacttgatgcagaagacttttctgcatcaaacaacagagcagattctctatatgttgtcagtccccagttttccatagcaccagactgaaagtcgggaatagcagcaagatcttgggaaggaagtaataaaatacattaccagtctctaagctatacattctcccaaacactaatcttcagaatggataggtgtttgaaccagctgcccgtgcataatcatggagctctagactggaagaacctttcacaatggagaaaaagaggcttagtgactcagcatcactt

SEQ ID NO: 18 - DNA, 692 bp, Homo sapiens
  variation (489)..(489): R = G/A
acatctataataaaattgatacttttttttttctttttttgagacggagtctcgctctgtcgcccaggctggagtgcagtggtgcgatctcggctcactgcaagctccgccttccgggttcacgccattctcctgcctcagcctcccgagtagctgggactacaggcgccagccacctcgccccgctaattttttgtatttttagtagagatggggtttcaccatgttagccaggatggtctcgatctcctgacctcatgatcggcccgcctcgacctcccgatacttttttaaaatgtaagatctggtggaaatatgtgaaacctaatcagtaagaataaaaattcaacatctgagtcttgtgtaacaaactaaatattcaagtatgtattacataataaatgccagtttgcaaaaatatgaactcattcaaacttaaagacttaaatctggattgcactgacctgctttatgctgtgattcttactrtgctatctgcatttctcataagactaaacagatttttactttctcctcagaataattcttttgccaaaagggtcctagaatgcttgtgctgctataacagtggccaagccgaactcagaggtggaggtgggtgacaggaggaggtgagaaatttatggccttgagggtgcttaaaatggtcctaagcaatttcagagccccag

SEQ ID NO: 19 - DNA, 201 bp, Homo sapiens
  variation (101)..(101): K = T/G
atgacacatggaattctgggctaacagttgcttccatctctacagggcaccttacttctggtaagaaaatacaacttaggctttttgagtagtcttttagkaattgcccattttaacccatcatactgaaaaaatcacatcaggtgttaagtttctggacaataagatatgccttatntcttccataggaaaataatagac

SEQ ID NO: 20 - DNA, 601 bp, Homo sapiens
  variation (301)..(301): R = G/A
ttctcaaacaaaaagttgtttcctggggtagttgtgcactctggaaaaacagtcactctgtggcctaaagtaaaggttaattttgcttccccccaccctttctcctttgagacctttgctttgagcagagtaaagagaatagtaattctggtatcaaatgaagactaatgcttggttaaaattatttttctttcctttcattagacaacagaggagacattggacttttattgggaatgatcgtctttgctgttatgttgtcaattctttctttgattgggatatttaacagatcattccraactgggtaggtttttgcagaatttctgttttctgatttagactacatgtatatgtatcaccaaaatttagtcatttcagttgtttactagaaaaatctgttaacatttttattcagataaaggaaaataaaaagaacaatgtttaataagtacttacccatgccaaactctctacaaatgtctttcctttaatcctcaaaatgaccctgccagaaaagcttcctggcctattttacaggtgacttaaatgaggcttaaagaggctaagtcctcagcccagaatcactgaacagtaagcc

SEQ ID NO: 21 - DNA, 648 bp, Homo sapiens
  variation (259)..(259): Y = C/T
ctgccaaacttttccattttgttatttaatttcttaaacattaacttcttaatttaatttgtttagttttatgagaactaaaatgctcattaaaatacacatttaaaaattccaatatttatatctcccatggctctttccagttaggggtcaattatatattatttttgtaggccgaattatttcttaactcacctctaaacatttgtatgtgaaaaattataagagcaatttgaggctttggaaaatgttctcttcytccacagaggattaacatttgcttctggcagcaagtttggccagaagtaggtcatcttaatgcaatctgggattaaaataatccaaagctaggcctcagttcttgtgaagtctgttctcattcacatttattcctgagatgatgcccttcaggatccccgctgaatgcctgaagtgtctaccagggaaccttttctagccttttctagctttgtcttttttttcctccacttttttttgtccctacagctcacaggacacctcgtggagctcctcagccaccagccactttgtcgggttcctctaaggggaaagagagtccaaataccaggctcacatatctaggttttctttttcttccaagacttggtcctgcaaattctgtcttcc

SEQ ID NO: 22 - DNA, 1484 bp, Homo sapiens
  variation (643)..(643): R = A/G
ctctctaactccttgcattatggggttgtaggaaaacctataaggtcattttcttgtgtgtgggtttttttattgttgttgctgcttattgtttgttttacatagagatgggttcgattcccaggctggtctcaaactcctgggctccagtgatcctcctgccttggtctcccaaagtgctgggattataggcatgagccaccatgcctggccctatacggtcatttatatgtggtaatcatcaatgcttttgccaaatatccattttagttttatccccttctgggcatatgtcaggattgcatttcttggtgtctttgtgatacggtagagccacatagccagccctggctggtgattttctttggcggatgatgttggagcagcagtgtaactgaaacacgtaccagtcacttgccacctgcagggtccaattcacaaaagtgaggtctggtctaaagaaagtgactttattccaatgtttagcttaggggaagtacaggctcttgcctttaagggtattgcttcacttttggggcagaaagcagggaattttaaaggggatttggcatgaatggcatgcatgggagggaatgagcaggtgcaaggtctaagtgactgactttggtgccgtatctaccargtggtcaagctgatgccattgcaagtcgtaaagtggccattgcctcaagatcccctccgggtgggagacagttctctcgcgggcgtactttaggttgtaaattgactgttgtctctcaggtgatctcttgatggaagagagctcccgctctggagcttctaagtcagcacatagataagcttgctgtgcagggagtgtcttaaggtctgtgtccttctgcacactcagggaaatccatttaccttttgcagctcctggaggccgccacattccctggctcatagccccttccttcttcaaagccagccacggctggctgagccttcctcagatcacatcactctgacacccactcttgttaacaaaagaccatgaggtacacagagaaaggaaaggagaagactttattttcagagggagaagcaatcgtggattgaggaacatagcttccagggacaaccgaaagtacacgccctacagaaggaagggggagctggtatatgtgtcttatagggcaagacttacatgcgtattgagcagggttggggacattctatgaatattcatgagggatggctggcacgtgcacagtgggtaaatgtctataacatacattccattcactttgggatggggtttcaggaattaaaatgaggtagaatttggctcttgatctcaaaaggtaaacagacactttgtgcacagtctctataagctaaatggctgtgactggcttgaggtctgcagccatttatcagtaaagaaagtttataagaccagtcctctgtccaatcagagttgtggtggggagggaggggagattggagactgggtatgcagg